SEQUENCE LISTING (20 sequences)

SEQ ID NO: 1 - protein, 106 aa, Artificial [sequence omitted]

SEQ ID NO: 2 - protein, 121 aa, Artificial [sequence omitted]

SEQ ID NO: 3 - protein, 106 aa, Artificial [sequence omitted]

SEQ ID NO: 4 - protein, 122 aa, Artificial [sequence omitted]

SEQ ID NO: 5 - protein, 107 aa, Artificial [sequence omitted]

SEQ ID NO: 6 - protein, 120 aa, Artificial [sequence omitted]

SEQ ID NO: 7 - protein, 111 aa, Artificial
  Synthetic
EIVLTQSPDSLAVSLGERATINCKASQSVDFDGDSYLNWYQQKPGQPPKVLIYAASTLQSGVPSRFSGSGSGTDFTLTINSLEAEDAATYYCQQSNEDPWTFGGGTKVEIK

SEQ ID NO: 8 - protein, 117 aa, Artificial
  Synthetic
QVQLVQSGAEVKKPGASVKVSCKVSGYTFTDYYITWVRQAPGQALEWMGWIYPGSGNTKYSQKFQGRFVFSVDTSASTAYLQISSLKAEDTAVYYCANYGNYWFAYWGQGTLVTVSS

SEQ ID NO: 9 - protein, 107 aa, Artificial [sequence omitted]

SEQ ID NO: 10 - protein, 330 aa, Homo sapiens [sequence omitted]

SEQ ID NO: 11 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 13 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 329 aa, Artificial
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 330 aa, Artificial
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 213 aa, Artificial
  Synthetic
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 451 aa, Artificial
  Synthetic
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 18 - protein, 218 aa, Artificial
  Synthetic
EIVLTQSPDSLAVSLGERATINCKASQSVDFDGDSYLNWYQQKPGQPPKVLIYAASTLQSGVPSRFSGSGSGTDFTLTINSLEAEDAATYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 447 aa, Artificial
  Synthetic
QVQLVQSGAEVKKPGASVKVSCKVSGYTFTDYYITWVRQAPGQALEWMGWIYPGSGNTKYSQKFQGRFVFSVDTSASTAYLQISSLKAEDTAVYYCANYGNYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 20 - protein, 46 aa, Artificial
  Synthetic
RCPEPKSCDTPPPCPRCPEPKSCDTPPPCPRCPEPKSCDTPPPCPR

(19) United States
(12) Patent Application Publication
(10) Pub. No.: US 2010/0317834 A1
(43) Pub. Date: Dec. 16, 2010

(54) IgG Immunoglobulin Variants with Optimized Effector Function

(75) Inventors: Gregory Alan Lazar, Los Angeles, CA (US); Bassil I. Dahiyat, Altadena, CA (US); Wei Dang, Pasadena, CA (US); Sher Bahadur Karki, Pomona, CA (US); Omid Vafa, Monrovia, CA (US)

Correspondence Address: MORGAN, LEWIS & BOCKIUS, LLP (SF), ONE MARKET SPEAR STREET TOWER, SAN FRANCISCO, CA, 94105, US

(73) Assignee: Xencor, Inc., Monrovia, CA (US)

(21) Appl. No.: 12/862,583

(22) Filed: Aug. 24, 2010

Related U.S. Application Data
(63) Continuation of application No. 11/256,060, filed on Oct. 21, 2005, now abandoned.
(60) Provisional application No. 60/621,387, filed on Oct. 21, 2004.
(60) Provisional application No. 60/629,068, filed on Nov. 18, 2004.
(60) Provisional application No. 60/652,968, filed on Feb. 14, 2005.
(60) Provisional application No. 60/659,004, filed on Mar. 3, 2005.
(60) Provisional application No. 60/723,294, filed on Oct. 3, 2005.

(51) Int. Cl. C07K 16/00

(52) U.S. Cl. 530/387.1

(57) ABSTRACT

The present application relates to optimized IgG immunoglobulin variants, engineering methods for their generation, and their application, particularly for therapeutic purposes.

The present application is a continuation of U.S. application Ser. No. 11/256,060, filed Oct. 21, 2005 which claims benefit under 35 U.S.C. §119(e) to U.S. Provisional Application Nos. 60/621,387, filed Oct. 21, 2004; 60/629,068, filed Nov. 18, 2004; 60/652,968, filed Feb. 14, 2005, and 60/659,004, filed Mar. 3, 2005, each of which is incorporated herein by reference in its entirety.

### FIELD

The present application relates to optimized IgG immunoglobulin variants, engineering methods for their generation, and their application, particularly for therapeutic purposes.

### BACKGROUND

Antibodies are immunological proteins that bind a specific antigen. In most mammals, including humans and mice, antibodies are constructed from paired heavy and light polypeptide chains. Each chain is made up of individual immunoglobulin (Ig) domains, and thus the generic term immunoglobulin is used for such proteins. Each chain is made up of two distinct regions, referred to as the variable and constant regions. The light and heavy chain variable regions show significant sequence diversity between antibodies, and are responsible for binding the target antigen. The constant regions show less sequence diversity, and are responsible for binding a number of natural proteins to elicit important biochemical events. In humans there are five different classes of antibodies including IgA (which includes subclasses IgA1 and IgA2), IgD, IgE, IgG (which includes subclasses IgG1, IgG2, IgG3, and IgG4), and IgM. The distinguishing features between these antibody classes are their constant regions, although subtler differences may exist in the V region. FIG. 1 shows an IgG1 antibody, used here as an example to describe the general structural features of immunoglobulins. IgG antibodies are tetrameric proteins composed of two heavy chains and two light chains. The IgG heavy chain is composed of four immunoglobulin domains linked from N- to C-terminus in the order VH-CH1-CH2-CH3, referring to the heavy chain variable domain, heavy chain constant domain 1, heavy chain constant domain 2, and heavy chain constant domain 3 respectively (also referred to as VH-Cγ1-Cγ2-Cγ3, referring to the heavy chain variable domain, constant gamma 1 domain, constant gamma 2 domain, and constant gamma 3 domain respectively). The IgG light chain is composed of two immunoglobulin domains linked from N- to C-terminus in the order VL-CL, referring to the light chain variable domain and the light chain constant domain respectively.

The variable region of an antibody contains the antigen binding determinants of the molecule, and thus determines the specificity of an antibody for its target antigen. The variable region is so named because it is the most distinct in sequence from other antibodies within the same class. The majority of sequence variability occurs in the complementarity determining regions (CDRs). There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens. A number of high-resolution structures are available for a variety of variable region fragments from different organisms, some unbound and some in complex with antigen. The sequence and structural features of antibody variable regions are well characterized (Morea et al., 1997, Biophys Chem 68:9-16; Morea et al., 2000, Methods 20:267-279), and the conserved features of antibodies have enabled the development of a wealth of antibody engineering techniques (Maynard et al., 2000, Annu Rev Biomed Eng 2:339-376). For example, it is possible to graft the CDRs from one antibody, for example a murine antibody, onto the framework region of another antibody, for example a human antibody. This process, referred to in the art as “humanization”, enables generation of less immunogenic antibody therapeutics from nonhuman antibodies. Fragments including the variable region can exist in the absence of other regions of the antibody, including for example the antigen binding fragment (Fab) including VH-Cγ1 and VH-CL, the variable fragment (Fv) including VH and VL, the single chain variable fragment (scFv) including VH and VL linked together in the same chain, as well as a variety of other variable region fragments (Little et al., 2000, Immunol Today 21:364-370).

The Fc region of an antibody interacts with a number of Fc receptors and ligands, imparting an array of important functional capabilities referred to as effector functions. For IgG the Fc region, as shown in FIG. 1, comprises Ig domains Cγ2 and Cγ3 and the N-terminal hinge leading into Cγ2. An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and γγ T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP). A number of structures have been solved of the extracellular domains of human FcγRs, including FcγRIIa (pdb accession code 1H9V)(Sondermann et al., 2001, J Mol Blot 309:737-749) (pdb accession code 1FCG)(Maxwell et al., 1999, Nat Struct Biol 6:437-442), FcγRIIb (pdb accession code 2FCB)(Sondermann et al., 1999, Embo J 18:1095-1103); and FcγRIIIb (pdb accession code 1 E4J)(Sondermann et al., 2000, Nature 406:267-273). All FcγRs bind the same region on Fc, at the N-terminal end of the Cγ2 domain and the preceding hinge, shown in FIG. 2. This interaction is well characterized structurally (Sondermann et al., 2001, J Mol Biol 309:737-749), and several structures of the human Fc bound to the extracellular domain of human FcγRIIIb have been solved (pdb accession code 1E4K) (Sondermann et al., 2000, Nature 406:267-273) (pdb accession codes 1IIS and 1IIX) (Radaev et al., 2001, J Biol Chem 276:16469-16477), as well as has the structure of the human IgE Fc/FcεRIα complex (pdb accession code 1F6A) (Garman et al., 2000, Nature 406:259-266).

The different IgG subclasses have different affinities for the FcγRs, with IgG1 and IgG3 typically binding substantially better to the receptors than IgG2 and IgG4 (Jefferis et al., 2002, Immunol Lett 82:57-65). All FcγRs bind the same region on IgG Fc, yet with different affinities: the high affinity binder FcγRI has a Kd for IgG1 of 10⁻⁸ M⁻¹, whereas the low affinity receptors FcγRII and FcγRIII generally bind at 10⁻⁶ and 10⁻⁵ respectively. The extracellular domains of FcγRIIIa and FcγRIIIb are 96% identical, however FcγRIIIb does not have a intracellular signaling domain. Furthermore, whereas FcγRI, FcγRIIa/c, and FcγRIIIa are positive regulators of immune complex-triggered activation, characterized by having an intracellular domain that has an immunoreceptor tyrosine-based activation motif (ITAM), FcγRIIb has an immunoreceptor tyrosine-based inhibition motif (ITIM) and is therefore inhibitory. Thus the former are referred to as activation receptors, and FcγRIIb is referred to as an inhibitory receptor. The receptors also differ in expression pattern and levels on different immune cells. Yet another level of complexity is the existence of a number of FcγR polymorphisms in the human proteome. A particularly relevant polymorphism with clinical significance is V158/F158 FcγRIIIa. Human IgG1 binds with greater affinity to the V158 allotype than to the F158 allotype. This difference in affinity, and presumably its effect on ADCC and/or ADCP, has been shown to be a significant determinant of the efficacy of the anti-CD20 antibody rituximab (Rituxan®, a registered trademark of IDEC Pharmaceuticals Corporation). Patients with the V158 allotype respond favorably to rituximab treatment; however, patients with the lower affinity F158 allotype respond poorly (Cartron et al., 2002, Blood 99:754-758). Approximately 10-20% of humans are V158/V158 homozygous, 45% are V158/F158 heterozygous, and 35-45% of humans are F158/F158 homozygous (Lehrnbecher et al., 1999, Blood 94:4220-4232; Cartron et al., 2002, Blood 99:754-758). Thus 80-90% of humans are poor responders, that is they have at least one allele of the F158 FcγRIIIa.

An overlapping but separate site on Fc, shown in FIG. 1, serves as the interface for the complement protein C1q. In the same way that Fc/FcγR binding mediates ADCC, Fc/C1q binding mediates complement dependent cytotoxicity (CDC). C1q forms a complex with the serine proteases C1r and C1s to form the C1 complex. C1q is capable of binding six antibodies, although binding to two IgGs is sufficient to activate the complement cascade. Similar to Fc interaction with FcγRs, different IgG subclasses have different affinity for C1q, with IgG1 and IgG3 typically binding substantially better to the FcγRs than IgG2 and IgG4 (Jefferis et al., 2002, Immunol Lett 82:57-65).

A site on Fc between the Cγ2 and Cγ3 domains, shown in FIG. 1, mediates interaction with the neonatal receptor FcRn, the binding of which recycles endocytosed antibody from the endosome back to the bloodstream (Raghavan et al., 1996, Annu Rev Cell Dev Biol 12:181-220; Ghetie et al., 2000, Annu Rev Immunol 18:739-766). This process, coupled with preclusion of kidney filtration due to the large size of the full length molecule, results in favorable antibody serum half-lives ranging from one to three weeks. Binding of Fc to FcRn also plays a key role in antibody transport. The binding site for FcRn on Fc is also the site at which the bacterial proteins A and G bind. The tight binding by these proteins is typically exploited as a means to purify antibodies by employing protein A or protein G affinity chromatography during protein purification. Thus the fidelity of this region on Fc is important for both the clinical properties of antibodies and their purification. Structures of the rat Fc/FcRn complex have been disclosed (Martin et al., 2001, Mol Cell 7:867-877). The complexes of Fc with proteins A and G have also been disclosed (Deisenhofer, 1981, Biochemistry 20:2361-2370; Sauer-Eriksson et al., 1995, Structure 3:265-278; Tashiro et al., 1995, Curr Opin Struct Biol 5:471-481).

One feature of the Fc region is the conserved N-linked glycosylation that occurs at N297, shown in FIG. 1. This carbohydrate, or oligosaccharide as it is sometimes referred, plays a critical structural and functional role for the antibody, and is one of the principle reasons that antibodies must be produced using mammalian expression systems. Umaña et al., 1999, Nat Biotechnol 17:176-180; Davies et al., 2001, Biotechnol Bioeng 74:288-294; Mimura et al., 2001, J Biol Chem 276:45539-45547; Radaev et al., 2001, J Biol Chem 276:16478-16483; Shields et al., 2001, J Biol Chem 276:6591-6604; Shields et al., 2002, J Biol Chem 277:26733-26740; Simmons et al., 2002, J Immunol Methods 263:133-147; Radaev et al., 2001, J Biol Chem 276:16469-16477; and Krapp et al., 2003, J Mol Biol 325:979-989).

Antibodies have been developed for therapeutic use. Representative publications related to such therapies include Chamow et al., 1996, Trends Biotechnol 14:52-60; Ashkenazi et al., 1997, Curr Opin Immunol 9:195-200, Cragg et al., 1999, Curr Opin Immunol 11:541-547; Glennie et al., 2000, Immunol Today 21:403-410, McLaughlin et al., 1998, J Clin Oncol 16:2825-2833, and Cobleigh et al., 1999, J Clin Oncol 17:2639-2648. Currently for anticancer therapy, any small improvement in mortality rate defines success. Certain IgG variants disclosed herein enhance the capacity of antibodies to destroy targeted cancer cells.

Anti-tumor potency of antibodies is via enhancement of their ability to mediate cytotoxic effector functions such as ADCC, ADCP, and CDC. Examples include Clynes et al., 1998, Proc Natl Acad Sci USA 95:652-656; Clynes et al., 2000, Nat Med 6:443-446; and Cartron et al., 2002, Blood 99:754-758.

Human IgG1 is the most commonly used antibody for therapeutic purposes, and the majority of engineering studies have been constructed in this context. The different isotypes of the IgG class however, including IgG1, IgG2, IgG3, and IgG4, have unique physical, biological, and clinical properties. There is a need in the art to design IgG2, IgG3, and IgG4 variants. There is a further need to design such variants to improve binding to an FcγR or enhance effector function as compared to native IgG polypeptides. The present application meets these and other needs.

### SUMMARY

The present application is directed to IgG2, IgG3, and IgG4 variants. Certain variants include isotopic amino acid modifications between IgG1, IgG2, IgG3, and IgG4. The variations can include isotopic modifications between in at least 2 domains, 3, domains, 3 domains, or 4 domains. Exchange domains can be CH1, CH2, hinge, and CH3 domains, CH1, CH2, and CH3 domains, or CH2 and CH3 domains.

Alternatively, certain specific modifications can be made to IgG2, IgG3, and IgG4 variants that are not found in any other IgG subclass. In certain embodiments, the variations can occur within only the Fc region of the IgG subclass, or only within one or more specific domains.

In additional aspects, IgG2, IgG3, and IgG4 variants that exhibit altered binding to an FcγR or enhances effector function as compared to native IgG polypeptides can be designed. For example, altered binding to an FcγR such as FcγRI, FcγRIIa, FcγRIIb, FcγRIIc, or FcγRIIIa can be designed. Alternatively, one or more effector functions (e.g. ADCC, ADCP, and CDC) can be designed.

In one aspect, the present application is directed to IgG2, IgG3, or IgG4 variants with one or more isotypic substitutions. In an embodiment, of such variants, the IgG2, IgG3, or IgG4 variant including an amino acid sequence having the formula:

ASTKGPSVFPLAP-X(131)-S-X(133)-STS-X(137)-X(138)-
TAALGCLVKDYFPEPVTVSWNSGALTSGVHTFPAVLQS
SGLYSLSSVVTVPSS-X(192)-X(193)-GT-X(196)-TY-X(199)-
CNV-X(203)-HKPSNTKVDK-X(214)-VE-X(217)-K-X(219)-
X(220)-X(221)-X(222)-X(223)-X(224)-X(225)-CP-
X(228)-CPAP-X(233)-X(234)-X(235)-X(236)-GPSVFLFPPK
PKDTLMISRTPEVTCVVVDVS-X(268)-EDPEV-X(274)-F-
X(276)-WYVDGVEVHNAKTKPREEQ-X(296)-NST-X(300)-
RVVSVLTV-X(309)-HQDWLNGKEYKCKVSNK-X(327)-LP-
X(330)-X(331)-IEKTISK-X(339)-KGQPREPQVYTLPPS-
X(355)-X(356)-E-X(358)-TKNQVSLTCLVKGFYPSDIAVE
WES-X(384)-GQPENNY-X(392)-TTPP-X(397)-LDSDGSF
FLYS-X(409)-LTVDKSRWQ-X(419)-GN-X(422)-FSCSVMH
EALHN-X(435)-X(436)-TQKSLSLS-X(445)-GK,

wherein
-X(131)- is selected from the group consisting of C and S;
-X(133)- is selected from the group consisting of R and K;
-X(137)- is selected from the group consisting of E and G;
-X(138)- is selected from the group consisting of S and G;
-X(192)- is selected from the group consisting of N and S;
-X(193)- is selected from the group consisting of F and L;
-X(196)- is selected from the group consisting of Q and K;
-X(199)- is selected from the group consisting of T and I;
-X(203)- is selected from the group consisting of D and N;
-X(214)- is selected from the group consisting of T, K and R;
-X(217)- is selected from the group consisting of R, P, L and S;
-X(219)- is selected from the group consisting of C, S, T and Y;
-X(220)- is selected from the group consisting of C, P and G;
-X(221)- is selected from the group consisting of no amino acid, D, L and the sequence LGD;
-X(222)- is selected from the group consisting of V, K, T and no amino acid;
-X(223)- is selected from the group consisting of no amino acid and T;
-X(224)- is selected from the group consisting of E, H and P;
-X(225)- is selected from the group consisting of no amino acid, T and P;
-X(228)- is selected from the group consisting of P, S, R, and the sequence

(SEQ ID NO: 20)

RCPEPKSCDTPPPCPRCPEPKSCDTPPPCPRCPEPKSCDTPPPCPR;

-X(233)- is selected from the group consisting of P and E;
-X(234)- is selected from the group consisting of V, L and F;
-X(235)- is selected from the group consisting of A and L;
-X(236)- is selected from the group consisting of no amino acid and G;
-X(268)- is selected from the group consisting of H and Q;
-X(274)- is selected from the group consisting of Q and K;
-X(276)- is selected from the group consisting of N and K;
-X(296)- is selected from the group consisting of F and Y;
-X(300)- is selected from the group consisting of F and Y;
-X(309)- is selected from the group consisting of V and L;
-X(327)- is selected from the group consisting of G and A;
-X(330)- is selected from the group consisting of A and S;
-X(331)- is selected from the group consisting of P and S;
-X(339)- is selected from the group consisting of T and A;
-X(355)- is selected from the group consisting of R and Q;
-X(356)- is selected from the group consisting of E and D;
-X(358)- is selected from the group consisting of M and L;
-X(384)- is selected from the group consisting of N and S;
-X(392)- is selected from the group consisting of K and N;
-X(397)- is selected from the group consisting of M and V;
-X(409)- is selected from the group consisting of K and R;
-X(419)- is selected from the group consisting of Q and E;
-X(422)- is selected from the group consisting of V and I;
-X(435)- is selected from the group consisting of H and R;
-X(436)- is selected from the group consisting of Y and F; and
-X(445)- is selected from the group consisting of P and L.

Variants of the formula can have at least 1, 2, 3, 4, 5, 6, 7, 8, 9, 10 or more amino acid modifications as compared to an amino acid sequence including SEQ ID NO:11, SEQ ID NO:12, or SEQ ID NO: 4. In a further embodiment, at least two of the modifications can be in different domains, at least three modifications can be in different domains, or at least four modifications can be in different domains.

In a further aspect, the present application is directed to an IgG2, IgG3, or IgG4 variant including at least 1, 2, 3, 4, 5, 6, 7, 8, 9, 10 or more amino acid modifications as compared to an amino acid sequence including SEQ ID NO:11, SEQ ID NO:12, or SEQ ID NO: 4. The modification can be at one or more positions selected from among positions 131, 133, 137, 138, 192, 193, 196, 199, 203, 214, 217, 219, 221, 222, 223, 224, 225, 226, 227, 228, 229, 230, 233, 234, 235, 236, 268, 274, 296, 300, 309, 327, 330, 335, 339, 356, 358, 384, 392, 397, 409, 419, 422, 435, 436 and 445. In further embodiments, at least two of the modifications can be in different domains, at least three modifications can be in different domains, or at least four modifications can be in different domains.

In another aspect, the present application is directed to an IgG2 variant including an amino acid sequence having the formula:

ASTKGPSVFPLAP-X(131)-S-X(133)-STS-X(137)-X(138)-
TAALGCLVKDYFPEPVTVSWNSGALTSGVHTFPAVLQSSGLYSLSSVV
TVPSS-X(192)-X(193)-GTQTY-X(199)-CNV-X(203)-HKPS
NTKVDK-X(214)-VE-X(217)-K-X(219)-C-X(221)-X(222)-
X(223)-X(224)-X(225)-CPPCPAP-X(233)-X(234)-X(235)-
X(236)-GPSVFLFPPKPKDTLMISRTPEVTCVVVDVSHEDPEV-
X(274)-FNWYVDGVEVHNAKTKPREEQ-X(296)-NST-X(300)-
RVVSVLTV-X(309)-HQDWLNGKEYKCKVSNK-X(327)-
LPAPIEKTISK-X(339)-KGQPREPQVYTLPPSR-X(356)-
E-X(358)-TKNQVSLTCLVKGFYPSDIAVEWESNGQPENNYKTTPP-
X(397)-LDSDGSFFLYSKLTVDKSRWQQGNVFSCSVMHEALHNH
YTQKSLSLSPGK,

wherein
X(131) is selected from the group consisting of C and S;
X(133) is selected from the group consisting of R and K;
X(137) is selected from the group consisting of E and G;
X(138) is selected from the group consisting of S and G;
X(192) is selected from the group consisting of N and S;
X(193) is selected from the group consisting of F and L;
X(199) is selected from the group consisting of T and I;
X(203) is selected from the group consisting of D and N;
X(214) is selected from the group consisting of T and K;
X(217) is selected from the group consisting of R and P;
X(219) is selected from the group consisting of C and S;
X(221) is selected from the group consisting of no amino acid and D;
X(222) is selected from the group consisting of V and K;
X(223) is selected from the group consisting of no amino acid and T;
X(224) is selected from the group consisting of E and H;
X(225) is selected from the group consisting of no amino acid and T;
X(233) is selected from the group consisting of P and E;
X(234) is selected from the group consisting of V and L;
X(235) is selected from the group consisting of A and L;
X(236) is selected from the group consisting of no amino acid and G;
X(274) is selected from the group consisting of Q and K;
X(296) is selected from the group consisting of F and Y;
X(300) is selected from the group consisting of F and Y;
X(309) is selected from the group consisting of V and L;
X(327) is selected from the group consisting of G and A;
X(339) is selected from the group consisting of T and A;
X(356) is selected from the group consisting of E and D;
X(358) is selected from the group consisting of M and L; and
X(397) is selected from the group consisting of M and V.

In various embodiments, the formula has at least 1, 2, 3, 4, 5, 6, 7, 8, 9, 10 or more amino acid modifications as compared to an amino acid sequence including SEQ ID NO:11. In additional embodiments, at least 2, 3, or 4 of the modifications are in different domains.

In another aspect, the present application is directed to an IgG2 variant including two or more amino acid modifications as compared to SEQ ID NO:11. The modification can be selected from among C131S, R133K, E137G, S138G, N192S, F193L, T199I, D203N, T214K, R217P, C219S, insertion of 221D, V222K, insertion of 223T, E224H, insertion of 225T, P233E, V234L, A235L, insertion of 236G, Q274K, F296Y, F300Y, V309L, G327A, T339A, E356D, M358L, and M397V. In various embodiments, the formula has at least 1, 2, 3, 4, 5, 6, 7, 8, 9, 10 or more amino acid modifications as compared to an amino acid sequence including SEQ ID NO:11. In additional embodiments, at least 2, 3, or 4 of the modifications are in different domains.

In a further variation, the present application is directed to an IgG2 variant including an amino acid sequence having the formula:

-ASTKGPSVFPLAP-X(131)-S-X(133)-STS-X(137)-X(138)-
TAALGCLVKDYFPEPVTVSWNSGALTSGVHTFPAVLQSSGLYSLSSVVT
VPSS-X(192)-X(193)-GT-X(196)-TY-X(199)-CNV-X(203)-
HKPSNTKVDK-X(214)-VE-X(217)-K-X(219)-X(220)-
X(221)-X(222)-X(223)-X(224)-X(225)-C-X(227)-
X(228)-C-X(230)-X(231)-X(232)-X(233)-X(234)-
X(235)-X(236)-X(237)-X(238)-X(239)-X(240)-
X(241)-L-X(243)-X(244)-X(245)-X(246)-X(247)-
K-X(249)-TLMIS-X(255)-TP-X(258)-V-X(260)-C-
X(262)-X(263)-X(264)-X(265)-X(266)-X(267)-X(268)-
X(269)-X(270)-X(271)-X(272)-X(273)-X(274)-X(275)-
X(276)-W-X(278)-V-X(280)-X(281)-X(282)-X(283)-
X(284)-X(285)-X(286)-A-X(288)-T-X(290)-X(291)-
X(292)-X(293)-X(294)-X(295)-X(296)-X(297)-X(298)-
X(299)-X(300)-X(301)-X(302)-X(303)-X(304)-X(305)-
LTV-X(309)-HQD-X(313)-LNG-X(317)-X(318)-Y-
X(320)-C-X(322)-X(323)-X(324)-X(325)-X(326)-
X(327)-X(328)-X(329)-X(330)-X(331)-X(332)-
X(333)-X(334)-X(335)-X(336)-X(337)-K-X(339)-
KGQPREPQVYTLPPS-X(355)-X(356)-E-X(358)-
TKNQVSLTCLVKGFYPSDIAVEWES-X(384)-
GQPENNY-X(392)-TTPP-X(397)-LDSDGSFFLYS-
X(409)-LTVDKSRWQ-X(419)-GN-X(422)-
FSCSVMHEALHN-X(435)-X(436)-TQKSLSLS-
X(445)-GK-,

, wherein
-X(131)- is selected from the group consisting of C and S;
-X(133)- is selected from the group consisting of R and K;
-X(137)- is selected from the group consisting of E and G;
-X(138)- is selected from the group consisting of S and G;
-X(192)- is selected from the group consisting of N and S;
-X(193)- is selected from the group consisting of F and L;
-X(196)- is selected from the group consisting of Q and K;
-X(199)- is selected from the group consisting of T and I;
-X(203)- is selected from the group consisting of D and N;
-X(214)- is selected from the group consisting of T, K and R;
-X(217)- is selected from the group consisting of R, P, L and S;
-X(219)- is selected from the group consisting of C, S, T and Y;
-X(220)- is selected from the group consisting of C, P and G;
-X(221)- is selected from the group consisting of no amino acid, D, K, L, Y and the sequence LGD;
-X(222)- is selected from the group consisting of V, K, T, no amino acid, E and Y;
-X(223)- is selected from the group consisting of no amino acid, T, E and K;
-X(224)- is selected from the group consisting of E, H, P and Y;
-X(225)- is selected from the group consisting of no amino acid, T, P, E, K and W;
-X(227)- is selected from the group consisting of P, E, G, K and Y;
-X(228)- is selected from the group consisting of P, S, R, E, G, K, Y, and the sequence

(SEQ ID NO: 20)

RCPEPKSCDTPPPCPRCPEPKSCDTPPPCPRCPEPKSCDTPPPCPR;

-X(230)- is selected from the group consisting of P, A, E, G and Y;
-X(231)- is selected from the group consisting of A, E, G, K, P and Y;
-X(232)- is selected from the group consisting of P, E, G, K and Y;
-X(233)- is selected from the group consisting of P, E, A, D, F, G, H, I, K, L, M, N, Q, R, S, T, V, W and Y;
-X(234)- is selected from the group consisting of V, L, F, D, E, F, G, H, I, K, M, N, P, Q, R, S, T, W and Y;
-X(235)- is selected from the group consisting of A, L, D, F, G, H, I, K, M, N, P, Q, R, S, T, V, W, and Y;
-X(236)- is selected from the group consisting of no amino acid, G, A, D, E, F, H, I, K, L, M, N, P, Q, R, S, T, V, W and Y;
-X(237)- is selected from the group consisting of G, D, E, F, H, I, K, L, M, N, P, Q, R, S, T, V, W and Y;
-X(238)- is selected from the group consisting of P, D, E, F, G, H, I, K, L, M, N, Q, R, S, T, V, W and Y;
-X(239)- is selected from the group consisting of S, D, E, F, G, H, I, K, L, M, N, P, Q, R, T, V, W and Y;
-X(240)- is selected from the group consisting of V, A, I, M and T;
-X(241)- is selected from the group consisting of F, D, E, L, R, S, W and Y;
-X(243)- is selected from the group consisting of F, E, H, L, Q, R, W, and Y;
-X(244)- is selected from the group consisting of P and H;
-X(245)- is selected from the group consisting of P and A;
-X(246)- is selected from the group consisting of, K, D, E, H and Y;
-X(247)- is selected from the group consisting of P, G and V;
-X(249)- is selected from the group consisting of D, H, Q and Y;
-X(255)- is selected from the group consisting of R and Y;
-X(258)- is selected from the group consisting of E, H, S and Y;
-X(260)- is selected from the group consisting of T, D, E, H and Y;
-X(262)- is selected from the group consisting of V, A, E, F, I and T;
-X(263)- is selected from the group consisting of V, A, I, M and T;
-X(264)- is selected from the group consisting of V, A, D, E, F, G, H, I, K, L, M, N, P, Q, R, S, T, W, and Y;
-X(265)- is selected from the group consisting of D, F, G, H, I, K, L, M, P, Q, R, S, T, V, W and Y;
-X(266)- is selected from the group consisting of V, A, I, M and T;
-X(267)- is selected from the group consisting of S, D, E, F, H, I, K, L, M, N, P, Q, R, V, W and Y;
-X(268)- is selected from the group consisting of H, Q, D, E, F, G, I, K, L, M, P, R, T, V and W;
-X(269)- is selected from the group consisting of E, F, G, H, I, K, L, M, N, P, R, S, T, V, W and Y;
-X(270)- is selected from the group consisting of D, F, G, H, I, L, M, P, Q, R, S, T, W and Y;
-X(271)- is selected from the group consisting of P, A, D, E, F, G, H, I, K, L, M, N, Q, R, S, T, V, W and Y;
-X(272)- is selected from the group consisting of E, D, F, G, H, I, K, L, M, P, R, S, T, V, W and Y;
-X(273)- is selected from the group consisting of V and I;
-X(274)- is selected from the group consisting of Q, K, D, E, F, G, H, I, L, M, N, P, R, T, V, W and Y;
-X(275)- is selected from the group consisting of F, L and W;
-X(276)- is selected from the group consisting of N, K, D, E, F, G, H, I, L, M, P, R, S, T, V, W and Y;
-X(278)- is selected from the group consisting of Y, D, E, G, H, I, K, L, M, N, P, Q, R, S, T, V and W;
-X(280)- is selected from the group consisting of D, G, K, L, P and W;
-X(281)- is selected from the group consisting of G, D, E, K, N, P, Q and Y;
-X(282)- is selected from the group consisting of V, E, G, K, P and Y;
-X(283)- is selected from the group consisting of E, G, H, K, L, P, R and Y;
-X(284)- is selected from the group consisting of V, D, E, L, N, Q, T and Y;
-X(285)- is selected from the group consisting of H, D, E, K, Q, W and Y;
-X(286)- is selected from the group consisting of N, E, G, P and Y;
-X(288)- is selected from the group consisting of K, D, E and Y;
-X(290)- is selected from the group consisting of K, D, H, L, N and W;
-X(291)- is selected from the group consisting of P, D, E, G, H, I, Q and T;
-X(292)- is selected from the group consisting of R, D, E, T and Y;
-X(293)- is selected from the group consisting of E, F, G, H, I, L, M, N, P, R, S, T, V, W and Y;
-X(294)- is selected from the group consisting of E, F, G, H, I, K, L, M, P, R, S, T, V, W and Y;
-X(295)- is selected from the group consisting of Q, D, E, F, G, H, I, M, N, P, R, S, T, V, W and Y;
-X(296)- is selected from the group consisting of F, Y, A, D, E, G, I, K, L, M, N, Q, R, S, T and V;
-X(297)- is selected from the group consisting of N, D, E, F, G, H, I, K, L, M, P, Q, R, S, T, V, W and Y;
-X(298)- is selected from the group consisting of S, E, F, H, I, K, M, Q, R, W and Y;
-X(299)- is selected from the group consisting of T, A, D, E, F, G, H, I, K, L, M, N, P, Q, R, S, V, W and Y;
-X(300)- is selected from the group consisting of F, Y, A, D, E, G, H, K, M, N, P, Q, R, S, T, V and W;
-X(301)- is selected from the group consisting of R, D, E, H and Y;
-X(302)- is selected from the group consisting of V and I;
-X(303)- is selected from the group consisting of V, D, E and Y;
-X(304)- is selected from the group consisting of S, D, H, L, N and T;
-X(305)- is selected from the group consisting of V, E, T and Y;
-X(309)- is selected from the group consisting of V and L;
-X(313)- is selected from the group consisting of W and F;
-X(317)- is selected from the group consisting of K, E and Q;
-X(318)- is selected from the group consisting of E, H, L, Q, R and Y;
-X(320)- is selected from the group consisting of K, D, F, G, H, I, L, N, P, S, T, V, W and Y;
-X(322)- is selected from the group consisting of K, D, F, G, H, I, P, S, T, V, W and Y;
-X(323)- is selected from the group consisting of V and I;
-X(324)- is selected from the group consisting of S, D, F, G, H, I, L, M, P, R, T, V, W and Y;
-X(325)- is selected from the group consisting of N, A, D, E, F, G, H, I, K, L, M, P, Q, R, S, T, V, W and Y;
-X(326)- is selected from the group consisting of K, I, L, P and T;
-X(327)- is selected from the group consisting of G, A, D, E, F, H, I, K, L, M, N, P, R, T, V, W and Y;
-X(328)- is selected from the group consisting of L, A, D, E, F, G, H, I, K, M, N, P, Q, R, S, T, V, W and Y;
-X(329)- is selected from the group consisting of P, D, E, F, G, H, I, K, L, M, N, Q, R, S, T, V, W and Y;
-X(330)- is selected from the group consisting of A, S, E, F, G, H, I, L, M, N, P, R, T, V, W and Y;
-X(331)- is selected from the group consisting of P, S, D, F, H, I, L, M, Q, R, T, V, W and Y;
-X(332)- is selected from the group consisting of I, A, D, E, F, H, K, L, M, N, P, Q, R, S, T, V, W and Y;
-X(333)- is selected from the group consisting of E, F, H, I, L, M, P, T and Y;
-X(334)- is selected from the group consisting of K, F, I, P and T;
-X(335)- is selected from the group consisting of T, D, F, G, H, I, L, M, N, P, R, S, V, W and Y;
-X(336)- is selected from the group consisting of I, E, K and Y;
-X(337)- is selected from the group consisting of S, E, H and N;
-X(339)- is selected from the group consisting of T and A;
-X(355)- is selected from the group consisting of R and Q;
-X(356)- is selected from the group consisting of E and D;
-X(358)- is selected from the group consisting of M and L;
-X(384)- is selected from the group consisting of N and S;
-X(392)- is selected from the group consisting of K and N;
-X(397)- is selected from the group consisting of M and V;
-X(409)- is selected from the group consisting of K and R;
-X(419)- is selected from the group consisting of Q and E;
-X(422)- is selected from the group consisting of V and I;
-X(435)- is selected from the group consisting of H and R;
-X(436)- is selected from the group consisting of Y and F; and
-X(445)- is selected from the group consisting of P and L.

In certain variations, a first modification is selected from among C131S, R133K, E137G, S138G, N192S, F193L, Q196K, T199I, D203N, T214K, T214R, R217P, R217L, R217S, C219S, C219T, C219Y, C220P, C220G, insertion of 221D, insertion of 221L, insertion of 221LGD, V222K, V222T, deletion of V222, insertion of 223T, E224H, E224P, insertion of 225T, insertion of 225P, P228R, substitution of P228 with RCPEPKSCDTPPPCPRCPEPKSCDTPPPCPRCPEPKSCDTPPPCPR (SEQ ID NO:20), P228S, P233E, V234L, V234F, A235L, insertion of 236G, H268Q, Q274K, N276K, F296Y, F300Y, V309L, G327A, A330S, P331S, T339A, R355Q, E356D, M358L, N384S, K392N, M397V, K409R, Q419E, V4221, H435R, Y436F, and P445L. In a further variation, a second modification is selected from among 221K, 221Y, 222E, 222Y, 223E, 223K, 224Y, 225E, 225K, 225W, 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N.

In another aspect, a first modification selected from among

C131S, R133K, E137G, S138G, N192S, F193L, Q196K, T199I, D203N, T214K, T214R, R217P, R217L, R217S, C219S, C219T, C219Y, C220P, C220G, insertion of 221D, insertion of 221L, insertion of 221LGD, V222K, V222T, deletion of V222, insertion of 223T, E224H, E224P, insertion of 225T, insertion of 225P, P228R, substitution of P228 with RCPEPKSCDTPPPCPRCPEPKSCDTPPPCPRCPEPKSCDTPPPCPR, P228S, P233E, V234L, V234F, A235L, insertion of 236G, H268Q, Q274K, N276K, F296Y, F300Y, V309L, G327A, A330S, P331S, T339A, R355Q, E356D, M358L, N384S, K392N, M397V, K409R, Q419E, V4221, H435R, Y436F, and P445L. In a further variation, a second modification selected from among 221K, 221Y, 222E, 222Y, 223E, 223K, 224Y, 225E, 225K, 225W, 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, 337N.

In another aspect, the present application is directed to an IgG2 variant including an amino acid sequence having the formula:

ASTKGPSVFPLAP-X(131)-S-X(133)-STS-X(137)-X(138)-
TAALGCLVKDYFPEPVTVSWNSGALTSGVHTFPAVLQSSGLYSLSSVV
TVPSS-X(192)-X(193)-GT-X(196)-TY-X(199)-CNV-
X(203)-HKPSNTKVDK-X(214)-VE-X(217)-K-X(219)-
X(220)-X(221)-X(222)-X(223)-X(224)-X(225)-C-
X(227)-X(228)-CPAP-X(233)-X(234)-X(235)-X(236)-
X(237)-P-X(239)-X(240)-FLFPP-X(246)-PKDTLMIS-
X(255)-TP-X(258)-V-X(260)-CVV-X(264)-DV-X(267)-
X(268)-ED-X(271)-X(272)-V-X(274)-F-X(276)-W-
X(278)-VD-X(281)-V-X(283)-X(284)-HNAKT-X(290)-
PR-X(293)-E-X(295)-X(296)-NST-X(300)-RVV-X(304)-
VLTV-X(309)-HQDWLNGKEYKCKV-X(324)-N-X(326)-
X(327)-X(328)-P-X(330)-X(331)-X(332)-X(333)-
X(334)-TISK-X(339)-KGQPREPQVYTLPPS-X(355)-X(356)-
E-X(358)-TKNQVSLTCLVKGFYPSDIAVEWES-X(384)-
GQPENNY-X(392)-TTPP-X(397)-LDSDGSFFLYS-
X(409)-LTVDKSRWQ-X(419)-GN-X(422)-FSCSVMHEALHN-
X(435)-X(436)-TQKSLSLS-X(445)-GK;

wherein
-X(131)- is selected from the group consisting of C and S;
-X(133)- is selected from the group consisting of R and K;
-X(137)- is selected from the group consisting of E and G;
-X(138)- is selected from the group consisting of S and G;
-X(192)- is selected from the group consisting of N and S;
-X(193)- is selected from the group consisting of F and L;
-X(196)- is selected from the group consisting of Q and K;
-X(199)- is selected from the group consisting of T and I;
-X(203)- is selected from the group consisting of D and N;
-X(214)- is selected from the group consisting of T, K and R;
-X(217)- is selected from the group consisting of R, P, L and S;
-X(219)- is selected from the group consisting of C, S, T and Y;
-X(220)- is selected from the group consisting of C, P and G;
-X(221)- is selected from the group consisting of no amino acid, D, K, L, and the sequence LGD;
-X(222)- is selected from the group consisting of V, K, T, and no amino acid;
-X(223)- is selected from the group consisting of no amino acid and T;
-X(224)- is selected from the group consisting of E, H and P;
-X(225)- is selected from the group consisting of no amino acid, T and P;
-X(227)- is selected from the group consisting of P and G;
-X(228)- is selected from the group consisting of P, S, R, and the sequence

(SEQ ID NO: 20)

RCPEPKSCDTPPPCPRCPEPKSCDTPPPCPRCPEPKSCDTPPPCPR;

-X(233)- is selected from the group consisting of P and E;
-X(234)- is selected from the group consisting of V, L, F, Y and I;
-X(235)- is selected from the group consisting of A, L, Y, I and D;
-X(236)- is selected from the group consisting of no amino acid, G, S and A;
-X(237)- is selected from the group consisting of G and D;
-X(239)- is selected from the group consisting of S, D, E, N, Q and T;
-X(240)- is selected from the group consisting of V, I and M;
-X(246)- is selected from the group consisting of K, H and Y;
-X(255)- is selected from the group consisting of R and Y;
-X(258)- is selected from the group consisting of E, H and Y;
-X(260)- is selected from the group consisting of T and H;
-X(264)- is selected from the group consisting of V, I, T and Y;
-X(267)- is selected from the group consisting of S, D and E;
-X(268)- is selected from the group consisting of H, Q, D and E;
-X(271)- is selected from the group consisting of P and G;
-X(272)- is selected from the group consisting of E, Y, H, R and I;
-X(274)- is selected from the group consisting of Q, K and E;
-X(276)- is selected from the group consisting of N and K;
-X(278)- is selected from the group consisting of Y and T;
-X(281)- is selected from the group consisting of G, D and E;
-X(283)- is selected from the group consisting of E, L and H;
-X(284)- is selected from the group consisting of V, E and D;
-X(290)- is selected from the group consisting of K and N;
-X(293)- is selected from the group consisting of E and R;
-X(295)- is selected from the group consisting of Q and E;
-X(296)- is selected from the group consisting of F and Y;
-X(300)- is selected from the group consisting of F and Y;
-X(304)- is selected from the group consisting of S and T;
-X(309)- is selected from the group consisting of V and L;
-X(324)- is selected from the group consisting of S, G and I;
-X(326)- is selected from the group consisting of K and T;
-X(327)- is selected from the group consisting of G, A and D;
-X(328)- is selected from the group consisting of L, A, F, I and T;
-X(330)- is selected from the group consisting of A, S, L, Y and I;
-X(331)- is selected from the group consisting of P and S;
-X(332)- is selected from the group consisting of I, D, E, N, Q and T;
-X(333)- is selected from the group consisting of E and Y;
-X(334)- is selected from the group consisting of K, F, I and T;
-X(339)- is selected from the group consisting of T and A;
-X(355)- is selected from the group consisting of R and Q;
-X(356)- is selected from the group consisting of E and D;
-X(358)- is selected from the group consisting of M and L;
-X(384)- is selected from the group consisting of N and S;
-X(392)- is selected from the group consisting of K and N;
-X(397)- is selected from the group consisting of M and V;
-X(409)- is selected from the group consisting of K and R;
-X(419)- is selected from the group consisting of Q and E;
-X(422)- is selected from the group consisting of V and I;
-X(435)- is selected from the group consisting of H and R;
-X(436)- is selected from the group consisting of Y and F; and
-X(445)- is selected from the group consisting of P and L.

In certain variations, a first modification is selected from among C131S, R133K, E137G, S138G, N192S, F193L, Q196K, T199I, D203N, T214K, T214R, R217P, R217L, R217S, C219S, C219T, C219Y, C220P, C220G, the insertion of 221D, the insertion of 221LGD, the insertion of 221L, V222K, V222T, the deletion of V222, the insertion of 223T, E224H, E224P, the insertion of 225T, the insertion of 225P, P228R, the substitution of RCPEPKSCDTPPPCPRCPEPKSCDTPPPCPRCPEPKSCDTPPPCPR (SEQ ID NO:20) for P228, P228S, P233E, V234L, V234F, A235L, the insertion of 236G, H268Q, Q274K, N276K, F296Y, F300Y, V309L, G327A, A330S, P331S, T339A, R355Q, E356D, M358L, N384S, K392N, M397V, K409R, Q419E, V422I, H435R, Y436F, and P445L. In further variations, a second modification is selected from among 221K, 227G, 234Y, 234I, 235Y, 235I, 235D, 236S, 236A, 237D, 239D, 239E, 239N, 239Q, 239T, 240I, 240M, 246H, 246Y, 255Y, 258H, 258Y, 260H, 264I, 264T, 264Y, 267D, 267E, 268D, 268E, 271G, 272Y, 272H, 272R, 272I, 274E, 278T, 281D, 281E, 283L, 283H, 284E, 284D, 290N, 293R, 295E, 304T, 324G, 324I, 326T, 327D, 328A, 328F, 328I, 328T, 330L, 330Y, 330I, 332D, 332E, 332N, 332Q, 332T, 333Y, 334F, 334I, and 334T.

In another aspect, the present application is directed to an IgG2 variant including an amino acid sequence having the formula:

ASTKGPSVFPLAPCSRSTSESTAALGCLVKDYFPEPVTVSWNSGALTSGVHTFPAVLQSSG
LYSLSSVVTVPSSNFGTQTYTCNVDHKPSNTKVDKTVERKCCVEC-X(227)-X(228)-CPAP-
X(233)-X(234)-X(235)-X(236)--X(237)-X(238)-X(239)-X(240)-X(241)-L-X(243)-X(244)-
X(245)-X(246)-X(247)-K-X(249)-TLMIS-X(255)-TP-X(258)-V-X(260)-C-X(262)-X(263)-
X(264)-X(265)-X(266)-X(267)-X(268)-X(269)-X(270)-X(271)-X(272)-X(273)-X(274)-X(275)-
X(276)-W-X(278)-V-X(280)-X(281)-X(282)-X(283)-X(284)-X(285)-X(286)-A-X(288)-T-X(290)-
X(291)-X(292)-X(293)-X(294)-X(295)-X(296)-X(297)-X(298)-X(299)-X(300)-X(301)-X(302)-
X(303)-X(304)-X(305)-LTV-X(309)-HQD-X(313)-LNG-X(317)-X(318)-Y-X(320)-C-X(322)-
X(323)-X(324)-X(325)-X(326)-X(327)-X(328)-X(329)-X(330)-X(331)-X(332)-X(333)-X(334)-
X(335)-X(336)-X(337)-K-X(339)-KGQPREPQVYTLPPS-X(355)-X(356)-E-X(358)-
TKNQVSLTCLVKGFYPSDIAVEWES-X(384)-GQPENNY-X(392)-TTPP-X(397)-
LDSDGSFFLYS-X(409)-LTVDKSRWQ-X(419)-GN-X(422)-FSCSVMHEALHN-X(435)-
X(436)-TQKSLSLS-X(445)-GK,

wherein
-X(237)- is selected from the group consisting of G, D, E, F, H, I, K, L, M, N, P, Q, R, S, T, V, W and Y;
-X(238)- is selected from the group consisting of P, D, E, F, G, H, I, K, L, M, N, Q, R, S, T, V, W and Y;
-X(239)- is selected from the group consisting of S, D, E, F, G, H, I, K, L, M, N, P, Q, R, T, V, W and Y;
-X(240)- is selected from the group consisting of V, A, I, M and T;
-X(241)- is selected from the group consisting of F, D, E, L, R, S, W and Y;
-X(243)- is selected from the group consisting of F, E, H, L, Q, R, and Y;
-X(244)- is selected from the group consisting of P and H;
-X(245)- is selected from the group consisting of P and A;
-X(246)- is selected from the group consisting of, K, D, E, H and Y;
-X(247)- is selected from the group consisting of P, G and V;
-X(249)- is selected from the group consisting of D, H, Q and Y;
-X(255)- is selected from the group consisting of R and Y;
-X(258)- is selected from the group consisting of E, H, S and Y;
-X(260)- is selected from the group consisting of T, D, E, H and Y;
-X(262)- is selected from the group consisting of V, A, E, F, I and T;
-X(263)- is selected from the group consisting of V, A, I, M and T;
-X(264)- is selected from the group consisting of V, A, D, E, F, G, H, I, K, L, M, N, P, Q, R, S, T, W, and Y;
-X(265)- is selected from the group consisting of D, F, G, H, I, K, L, M, P, Q, R, S, T, V, W and Y;
-X(266)- is selected from the group consisting of V, A, I, M and T;
-X(267)- is selected from the group consisting of S, D, E, F, H, I, K, L, M, N, P, Q, R, V, W and Y;
-X(268)- is selected from the group consisting of H, Q, D, E, F, G, I, K, L, M, P, R, T, V and W;
-X(269)- is selected from the group consisting of E, F, G, H, I, K, L, M, N, P, R, S, T, V, W and Y;
-X(270)- is selected from the group consisting of D, F, G, H, I, L, M, P, Q, R, S, T, W and Y;
-X(271)- is selected from the group consisting of P, A, D, E, F, G, H, I, K, L, M, N, Q, R, S, T, V, W and Y;
-X(272)- is selected from the group consisting of E, D, F, G, H, I, K, L, M, P, R, S, T, V, W and Y;
-X(273)- is selected from the group consisting of V and I;
-X(274)- is selected from the group consisting of Q, K, D, E, F, G, H, I, L, M, N, P, R, T, V, W and Y;
-X(275)- is selected from the group consisting of FL and W;
-X(276)- is selected from the group consisting of N, K, D, E, F, G, H, I, L, M, P, R, S, T, V, W and Y;
-X(278)- is selected from the group consisting of Y, D, E, G, H, I, K, L, M, N, P, Q, R, S, T, V and W;
-X(280)- is selected from the group consisting of D, G, K, L, P and W;
-X(281)- is selected from the group consisting of G, D, E, K, N, P, Q and Y;
-X(282)- is selected from the group consisting of V, E, G, K, P and Y;
-X(283)- is selected from the group consisting of E, G, H, K, L, P, R and Y;
-X(284)- is selected from the group consisting of V, D, E, L, N, Q, T and Y;
-X(285)- is selected from the group consisting of H, D, E, K, Q, W and Y;
-X(286)- is selected from the group consisting of N, E, G, P and Y;
-X(288)- is selected from the group consisting of K, D, E and Y;
-X(290)- is selected from the group consisting of K, D, H, L, N and W;
-X(291)- is selected from the group consisting of P, D, E, G, H, I, Q and T;
-X(292)- is selected from the group consisting of R, D, E, T and Y;
-X(293)- is selected from the group consisting of E, F, G, H, I, L, M, N, P, R, S, T, V, W and Y;
-X(294)- is selected from the group consisting of E, F, G, H, I, K, L, M, P, R, S, T, V, W and Y;
-X(295)- is selected from the group consisting of Q, D, E, F, G, H, I, M, N, P, R, S, T, V, W and Y;
-X(296)- is selected from the group consisting of F, Y, A, D, E, G, I, K, L, M, N, Q, R, S, T and V;
-X(297)- is selected from the group consisting of N, D, E, F, G, H, I, K, L, M, P, Q, R, S, T, V, W and Y;
-X(298)- is selected from the group consisting of S, E, F, H, I, K, M, Q, R, W and Y;
-X(299)- is selected from the group consisting of T, A, D, E, F, G, H, I, K, L, M, N, P, Q, R, S, V, W and Y;
-X(300)- is selected from the group consisting of F, Y, A, D, E, G, H, K, M, N, P, Q, R, S, T, V and W;
-X(301)- is selected from the group consisting of R, D, E, H and Y;
-X(302)- is selected from the group consisting of V and I;
-X(303)- is selected from the group consisting of V, D, E and Y;
-X(304)- is selected from the group consisting of S, D, H, L, N and T;
-X(305)- is selected from the group consisting of V, E, T and Y;
-X(309)- is selected from the group consisting of V and L;
-X(313)- is selected from the group consisting of W and F;
-X(317)- is selected from the group consisting of K, E and Q;
-X(318)- is selected from the group consisting of E, H, L, Q, R and Y;
-X(320)- is selected from the group consisting of K, D, F, G, H, I, L, N, P, S, T, V, W and Y;
-X(322)- is selected from the group consisting of K, D, F, G, H, I, P, S, T, V, W and Y;
-X(323)- is selected from the group consisting of V and I;
-X(324)- is selected from the group consisting of S, D, F, G, H, I, L, M, P, R, T, V, W and Y;
-X(325)- is selected from the group consisting of N, A, D, E, F, G, H, I, K, L, M, P, Q, R, S, T, V, W and Y;
-X(326)- is selected from the group consisting of K, I, L, P and T;
-X(327)- is selected from the group consisting of G, A, D, E, F, H, I, K, L, M, N, P, R, T, V, W and Y;
-X(328)- is selected from the group consisting of L, A, D, E, F, G, H, I, K, M, N, P, Q, R, S, T, V, W and Y;
-X(329)- is selected from the group consisting of P, D, E, F, G, H, I, K, L, M, N, Q, R, S, T, V, W and Y;
-X(330)- is selected from the group consisting of A, S, E, F, G, H, I, L, M, N, P, R, T, V, W and Y;
-X(331)- is selected from the group consisting of P, S, D, F, H, I, L, M, Q, R, T, V, W and Y;
-X(332)- is selected from the group consisting of I, A, D, E, F, H, K, L, M, N, P, Q, R, S, T, V, W and Y;
-X(333)- is selected from the group consisting of E, F, H, I, L, M, P, T and Y;
-X(334)- is selected from the group consisting of K, F, I, P and T;
-X(335)- is selected from the group consisting of T, D, F, G, H, I, L, M, N, P, R, S, V, W and Y;
-X(336)- is selected from the group consisting of I, E, K and Y;
-X(337)- is selected from the group consisting of S, E, H and N;
-X(339)- is selected from the group consisting of T and A;
-X(355)- is selected from the group consisting of R and Q;
-X(356)- is selected from the group consisting of E and D;
-X(358)- is selected from the group consisting of M and L;
-X(384)- is selected from the group consisting of N and S;
-X(392)- is selected from the group consisting of K and N;
-X(397)- is selected from the group consisting of M and V;
-X(409)- is selected from the group consisting of K and R;
-X(419)- is selected from the group consisting of Q and E;
-X(422)- is selected from the group consisting of V and I;
-X(435)- is selected from the group consisting of H and R;
-X(436)- is selected from the group consisting of Y and F; and
-X(445)- is selected from the group consisting of P and L.

In certain variations, a first modification is selected from among P228R, substitution of P228 with RCPEPKSCDTPPPCPRCPEPKSCDTPPPCPRCPEPKSCDTPPPCPR (SEQ ID NO:20), P228S, P233E, V234L, V234F, A235L, insertion of 236G, H268Q, Q274K, N276K, F296Y, F300Y, V309L, G327A, A330S, P331S, T339A, R355Q, E356D, M358L, N384S, K392N, M397V, K409R, Q419E, V4221, H435R, Y436F, and P445L. In additional variations, a second modification is selected from among 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N. In certain variations, X(227) is P and X(228) is P.

In a further aspect, the present application is directed to an IgG2 variant amino acid sequence including at least two modifications as compared to SEQ ID. NO:2. In certain variations, a first modification is selected from among P228R, substitution of P228 with RCPEPKSCDTPPPCPRCPEPKSCDTPPPCPRCPEPKSCDTPPPCPR (SEQ ID NO:20), P228S, P233E, V234L, V234F, A235L, insertion of 236G, H268Q, Q274K, N276K, F296Y, F300Y, V309L, G327A, A330S, P331S, T339A, R355Q, E356D, M358L, N384S, K392N, M397V, K409R, Q419E, V4221, H435R, Y436F, and P445L. In further variations, a second modification is selected from among 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 2971, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N.

In a further aspect, the application is directed to an IgG2 variant including an amino acid sequence having the formula

ASTKGPSVFPLAPCSRSTSESTAALGCLVKDYFPEPVTVSWNSGALTSGVHTFPAVLQSSG
LYSLSSVVTVPSSNFGTQTYTCNVDHKPSNTKVDKTVERKCCVEC-X(227)-X(228)-CPAP-
X(233)-X(234)-X(235)-X(236)-X(237)-P-X(239)-X(240)-FLFPP-X(246)-PKDTLMIS-X(255)-
TP-X(258)-V-X(260)-CVV-X(264)-DV-X(267)-X(268)-ED-X(271)-X(272)-V-X(274)-F-X(276)-
W-X(278)-VD-X(281)-V-X(283)-X(284)-HNAKT-X(290)-PR-X(293)-E-X(295)-X(296)-NST-
X(300)-RVV-X(304)-VLTV-X(309)-HQDWLNGKEYKCKV-X(324)-N-X(326)-X(327)-X(328)-P-
X(330)-X(331)-X(332)-X(333)-X(334)-TISK-X(339)-KGQPREPQVYTLPPS-X(355)-X(356)-E-
X(358)-TKNQVSLTCLVKGFYPSDIAVEWES-X(384)-GQPENNY-X(392)-TTPP-X(397)-
LDSDGSFFLYS-X(409)-LTVDKSRWQ-X(419)-GN-X(422)-FSCSVMHEALHN-X(435)-
X(436)-TQKSLSLS-X(445)-GK

wherein
-X(237)- is selected from the group consisting of G and D;
-X(239)- is selected from the group consisting of S, D, E, N, Q and T;
-X(240)- is selected from the group consisting of V, I and M;
-X(246)- is selected from the group consisting of K, H and Y;
-X(255)- is selected from the group consisting of R and Y;
-X(258)- is selected from the group consisting of E, H and Y;
-X(260)- is selected from the group consisting of T and H;
-X(264)- is selected from the group consisting of V, I, T and Y;
-X(267)- is selected from the group consisting of S, D and E;
-X(268)- is selected from the group consisting of H, Q, D and E;
-X(271)- is selected from the group consisting of P and G;
-X(272)- is selected from the group consisting of E, Y, H, R and I;
-X(274)- is selected from the group consisting of Q, K and E;
-X(276)- is selected from the group consisting of N and K;
-X(278)- is selected from the group consisting of Y and T;
-X(281)- is selected from the group consisting of G, D and E;
-X(283)- is selected from the group consisting of E, L and H;
-X(284)- is selected from the group consisting of V, E and D;
-X(290)- is selected from the group consisting of K and N;
-X(293)- is selected from the group consisting of E and R;
-X(295)- is selected from the group consisting of Q and E;
-X(296)- is selected from the group consisting of F and Y;
-X(300)- is selected from the group consisting of F and Y;
-X(304)- is selected from the group consisting of S and T;
-X(309)- is selected from the group consisting of V and L;
-X(324)- is selected from the group consisting of S, G and I;
-X(326)- is selected from the group consisting of K and T;
-X(327)- is selected from the group consisting of G, A and D;
-X(328)- is selected from the group consisting of L, A, F, I and T;
-X(330)- is selected from the group consisting of A, S, L, Y and I;
-X(331)- is selected from the group consisting of P and S;
-X(332)- is selected from the group consisting of I, D, E, N, Q and T;
-X(333)- is selected from the group consisting of E and Y;
-X(334)- is selected from the group consisting of K, F, I and T;
-X(339)- is selected from the group consisting of T and A;
-X(355)- is selected from the group consisting of R and Q;
-X(356)- is selected from the group consisting of E and D;
-X(358)- is selected from the group consisting of M and L;
-X(384)- is selected from the group consisting of N and S;
-X(392)- is selected from the group consisting of K and N;
-X(397)- is selected from the group consisting of M and V;
-X(409)- is selected from the group consisting of K and R;
-X(419)- is selected from the group consisting of Q and E;
-X(422)- is selected from the group consisting of V and I;
-X(435)- is selected from the group consisting of H and R;
-X(436)- is selected from the group consisting of Y and F;
-X(445)- is selected from the group consisting of P and L;

In certain variations, a first modification is selected from among P228R, the substitution of RCPEPKSCDTPPPCPRCPEPKSCDTPPPCPRCPEPKSCDTPPPCPR (SEQ ID NO:20) for P228, P228S, P233E, V234L, V234F, A235L, the insertion of 236G, H268Q, K274Q, N276K, Y296F, Y300F, L309V, A327G, A330S, P331S, A339T, R355Q, D356E, L358M, N384S, K392N, V397M, K409R, Q419E, V422I, H435R, Y436F, and P445L. In additional variations, a second modification is selected from among 227G, 234Y, 234I, 235Y, 235I, 235D, 236S, 236A, 237D, 239D, 239E, 239N, 239Q, 239T, 240I, 240M, 246H, 246Y, 255Y, 258H, 258Y, 260H, 264I, 264T, 264Y, 267D, 267E, 268D, 268E, 271G, 272Y, 272H, 272R, 272I, 274E, 278T, 281D, 281E, 283L, 283H, 284E, 284D, 290N, 293R, 295E, 304T, 324G, 324I, 326T, 327D, 328A, 328F, 328I, 328T, 330L, 330Y, 330I, 332D, 332E, 332N, 332Q, 332T, 333Y, 334F, 334I, and 334T.

In another aspect, the present application is directed to an IgG2 variant including an amino acid sequence having the formula:

ASTKGPSVFPLAPCSRSTSESTAALGCLVKDYFPEPVTVSWNSGALTSGVHTFPAVLQSSG
LYSLSSVVTVPSSNFGTQTYTCNVDHKPSNTKVDKTVERKCC-X(221)-X(222)-X(223)-
X(224)-X(225)-C-X(227)-X(228)-C-X(230)-X(231)-X(232)-ELLGG-X(238)-X(239)-X(240)-
X(241)-L-X(243)-X(244)-X(245)-X(246)-X(247)-K-X(249)-TLMIS-X(255)-TP-X(258)-V-
X(260)-C-X(262)-X(263)-X(264)-X(265)-X(266)-X(267)-X(268)-X(269)-X(270)-X(271)-
X(272)-X(273)-X(274)-X(275)-X(276)-W-X(278)-V-X(280)-X(281)-X(282)-X(283)-X(284)-
X(285)-X(286)-A-X(288)-T-X(290)-X(291)-X(292)-X(293)-X(294)-X(295)-X(296)-X(297)-
X(298)-X(299)-X(300)-X(301)-X(302)-X(303)-X(304)-X(305)-LTVVHQD-X(313)-LNG-X(317)-
X(318)-Y-X(320)-C-X(322)-X(323)-X(324)-X(325)-X(326)-X(327)-X(328)-X(329)-X(330)-
X(331)-X(332)-X(333)-X(334)-X(335)-X(336)-X(337)-
KTKGQPREPQVYTLPPSREEMTKNQVSLTCLVKGFYPSDIAVEWESNGQPENNYKTTPPM
LDSDGSFFLYSKLTVDKSRWQQGNVFSCSVMHEALHNHYTQKSLSLSPGK-,

wherein
-X(221)- is selected from the group consisting of no amino acid, K and Y;
-X(222)- is selected from the group consisting of V, E and Y;
-X(223)- is selected from the group consisting of no amino acid, E and K;
-X(224)- is selected from the group consisting of E and Y;
-X(225)- is selected from the group consisting of no amino acid, E, K and W;
-X(227)- is selected from the group consisting of P, E, G, K and Y;
-X(228)- is selected from the group consisting of P, E, G, K and Y;
-X(230)- is selected from the group consisting of P, A, E, G and Y;
-X(231)- is selected from the group consisting of A, E, G, K, P and Y;
-X(232)- is selected from the group consisting of P, E, G, K and Y;
-X(233)- is selected from the group consisting of P, A, D, F, G, H, I, K, L, M, N, Q, R, S, T, V, W and Y;
-X(234)- is selected from the group consisting of V, D, E, F, G, H, I, K, M, N, P, Q, R, S, T, W and Y;
-X(235)- is selected from the group consisting of A, D, F, G, H, I, K, M, N, P, Q, R, S, T, V, W and Y;
-X(236)- is selected from the group consisting of no amino acid, A, D, E, F, H, I, K, L, M, N, P, Q, R, S, T, V, W and Y;
-X(237)- is selected from the group consisting of G, D, E, F, H, I, K, L, M, N, P, Q, R, S, T, V, W and Y;
-X(238)- is selected from the group consisting of P, D, E, F, G, H, I, K, L, M, N, Q, R, S, T, V, W and Y;
-X(239)- is selected from the group consisting of S, D, E, F, G, H, I, K, L, M, N, P, Q, R, T, V, W and Y;
-X(240)- is selected from the group consisting of V, A, I, M and T;
-X(241)- is selected from the group consisting of F, D, E, L, R, S, W and Y;
-X(243)- is selected from the group consisting of F, E, H, L, Q, R, W and Y;
-X(244)- is selected from the group consisting of P and H;
-X(245)- is selected from the group consisting of P and A;
-X(246)- is selected from the group consisting of K, D, E, H and Y;
-X(247)- is selected from the group consisting of P, G and V;
-X(249)- is selected from the group consisting of D, H, Q and Y;
-X(255)- is selected from the group consisting of R, E and Y;
-X(258)- is selected from the group consisting of E, H, S and Y;
-X(260)- is selected from the group consisting of T, D, E, H and Y;
-X(262)- is selected from the group consisting of V, A, E, F, I and T;
-X(263)- is selected from the group consisting of V, A, I, M and T;
-X(264)- is selected from the group consisting of V, A, D, E, F, G, H, I, K, L, M, N, P, Q, R, S, T, W and Y;
-X(265)- is selected from the group consisting of D, F, G, H, I, K, L, M, P, Q, R, S, T, V, W and Y;
-X(266)- is selected from the group consisting of V, A, I, M and T;
-X(267)- is selected from the group consisting of S, D, E, F, H, I, K, L, M, N, P, Q, R, V, W and Y;
-X(268)- is selected from the group consisting of H, D, E, F, G, I, K, L, M, P, R, T, V and W;
-X(269)- is selected from the group consisting of E, F, G, H, I, K, L, M, N, P, R, S, T, V, W and Y;
-X(270)- is selected from the group consisting of D, F, G, H, I, L, M, P, Q, R, S, T, W and Y;
-X(271)- is selected from the group consisting of P, A, D, E, F, G, H, I, K, L, M, N, Q, R, S, T, V, W and Y;
-X(272)- is selected from the group consisting of E, D, F, G, H, I, K, L, M, P, R, S, T, V, W and Y;
-X(273)- is selected from the group consisting of V and I;
-X(274)- is selected from the group consisting of Q, D, E, F, G, H, I, L, M, N, P, R, T, V, W and Y;
-X(275)- is selected from the group consisting of F, L and W;
-X(276)- is selected from the group consisting of N, D, E, F, G, H, I, L, M, P, R, S, T, V, W and Y;
-X(278)- is selected from the group consisting of Y, D, E, G, H, I, K, L, M, N, P, Q, R, S, T, V and W;
-X(280)- is selected from the group consisting of D, G, K, L, P and W;
-X(281)- is selected from the group consisting of G, D, E, K, N, P, Q and Y;
-X(282)- is selected from the group consisting of V, E, G, K, P and Y;
-X(283)- is selected from the group consisting of E, G, H, K, L, P, R and Y;
-X(284)- is selected from the group consisting of V, D, E, L, N, Q, T and Y;
-X(285)- is selected from the group consisting of H, D, E, K, Q, W and Y;
-X(286)- is selected from the group consisting of N, E, G, P and Y;
-X(288)- is selected from the group consisting of K, D, E and Y;
-X(290)- is selected from the group consisting of K, D, H, L, N and W;
-X(291)- is selected from the group consisting of P, D, E, G, H, I, Q and T;
-X(292)- is selected from the group consisting of R, D, E, T and Y;
-X(293)- is selected from the group consisting of E, F, G, H, I, L, M, N, P, R, S, T, V, W and Y;
-X(294)- is selected from the group consisting of E, F, G, H, I, K, L, M, P, R, S, T, V, W and Y;
-X(295)- is selected from the group consisting of Q, D, E, F, G, H, I, M, N, P, R, S, T, V, W and Y;
-X(296)- is selected from the group consisting of F, A, D, E, G, I, K, L, M, N, Q, R, S, T and V;
-X(297)- is selected from the group consisting of N, D, E, F, G, H, I, K, L, M, P, Q, R, S, T, V, W and Y;
-X(298)- is selected from the group consisting of S, E, F, H, I, K, M, Q, R, W and Y;
-X(299)- is selected from the group consisting of T, A, D, E, F, G, H, I, K, L, M, N, P, Q, R, S, V, W and Y;
-X(300)- is selected from the group consisting of F, A, D, E, G, H, K, M, N, P, Q, R, S, T, V and W;
-X(301)- is selected from the group consisting of R, D, E, H and Y;
-X(302)- is selected from the group consisting of V and I;
-X(303)- is selected from the group consisting of V, D, E and Y;
-X(304)- is selected from the group consisting of S, D, H, L, N and T;
-X(305)- is selected from the group consisting of V, E, T and Y;
-X(313)- is selected from the group consisting of W and F;
-X(317)- is selected from the group consisting of K, E and Q;
-X(318)- is selected from the group consisting of E, H, L, Q, R and Y;
-X(320)- is selected from the group consisting of K, D, F, G, H, I, L, N, P, S, T, V, W and Y;
-X(322)- is selected from the group consisting of K, D, F, G, H, I, P, S, T, V, W and Y;
-X(323)- is selected from the group consisting of V and I;
-X(324)- is selected from the group consisting of S, D, F, G, H, I, L, M, P, R, T, V, W and Y;
-X(325)- is selected from the group consisting of N, A, D, E, F, G, H, I, K, L, M, P, Q, R, S, T, V, W and Y;
-X(326)- is selected from the group consisting of K, I, L, P and T;
-X(327)- is selected from the group consisting of A, G, D, E, F, H, I, K, L, M, N, P, R, T, V, W and Y;
-X(328)- is selected from the group consisting of L, A, D, E, F, G, H, I, K, M, N, P, Q, R, S, T, V, W and Y;
-X(329)- is selected from the group consisting of P, D, E, F, G, H, I, K, L, M, N, Q, R, S, T, V, W and Y;
-X(330)- is selected from the group consisting of A, E, F, G, H, I, L, M, N, P, R, T, V, W and Y;
-X(331)- is selected from the group consisting of P, D, F, H, I, L, M, Q, R, T, V, W and Y;
-X(332)- is selected from the group consisting of I, A, D, E, F, H, K, L, M, N, P, Q, R, S, T, V, W and Y;
-X(333)- is selected from the group consisting of E, F, H, I, L, M, P, T and Y;
-X(334)- is selected from the group consisting of K, F, I, P and T;
-X(335)- is selected from the group consisting of T, D, F, G, H, I, L, M, N, P, R, S, V, W and Y;
-X(336)- is selected from the group consisting of I, E, K and Y; and
-X(337)- is selected from the group consisting of S, E, H and N

The variant differs from SEQ ID. NO:2 by at least one amino acid In a further aspect, X(327) is A.

In another aspect, the present application is directed to an IgG2 variant including an amino acid sequence having the formula:

ASTKGPSVFPLAPCSRSTSESTAALGCLVKDYFPEPVTVSWNSGALTSGVHTFPAVLQSSG
LYSLSSVVTVPSSNFGTQTYTCNVDHKPSNTKVDKTVERKCC-X(221)-VEC-X(227)-
PCPAPELLGGP-X(239)-X(240)-FLFPP-X(246)-PKDTLMIS-X(255)-TP-X(258)-V-X(260)-
CVV-X(264)-DV-X(267)-X(268)-ED-X(271)-X(272)-V-X(274)-FNW-X(278)-VD-X(281)-V-
X(283)-X(284)-HNAKT-X(290)-PR-X(293)-E-X(295)-FNSTFRVV-X(304)-
VLTVVHQDWLNGKEYKCKV-X(324)-N-X(326)-X(327)-X(328)-P-X(330)-P-X(332)-X(333)-
X(334)-
TISKTKGQPREPQVYTLPPSREEMTKNQVSLTCLVKGFYPSDIAVEWESNGQPENNYKTTP
PMLDSDGSFFLYSKLTVDKSRWQQGNVFSCSVMHEALHNHYTQKSLSLSPGK,

wherein
-X(221)- is selected from the group consisting of no amino acid and K;
-X(227)- is selected from the group consisting of P and G;
-X(237)- is selected from the group consisting of G and D;
-X(239)- is selected from the group consisting of S, D, E, N, Q and T;
-X(240)- is selected from the group consisting of V, I and M;
-X(246)- is selected from the group consisting of K, H and Y;
-X(255)- is selected from the group consisting of R and Y;
-X(258)- is selected from the group consisting of E, H and Y;
-X(260)- is selected from the group consisting of T and H;
-X(264)- is selected from the group consisting of V, I, T and Y;
-X(267)- is selected from the group consisting of S, D and E;
-X(268)- is selected from the group consisting of H, D and E;
-X(271)- is selected from the group consisting of P and G;
-X(272)- is selected from the group consisting of E, Y, H, R and I;
-X(274)- is selected from the group consisting of Q and E;
-X(278)- is selected from the group consisting of Y and T;
-X(281)- is selected from the group consisting of G, D and E;
-X(283)- is selected from the group consisting of E, L and H;
-X(284)- is selected from the group consisting of V, E and D;
-X(290)- is selected from the group consisting of K and N;
-X(293)- is selected from the group consisting of E and R;
-X(295)- is selected from the group consisting of Q and E;
-X(304)- is selected from the group consisting of S and T;
-X(324)- is selected from the group consisting of S, G and I;
-X(326)- is selected from the group consisting of K and T;
-X(327)- is selected from the group consisting of A, G and D;
-X(328)- is selected from the group consisting of L, A, F, I and T;
-X(330)- is selected from the group consisting of A, L, Y and I;
-X(332)- is selected from the group consisting of I, D, E, N, Q and T;
-X(333)- is selected from the group consisting of E and Y; and
-X(334)- is selected from the group consisting of K, F, I and T.

In certain variations, at least one of the positions is different from the sequence of SEQ ID NO:5. In a further variation, X(327) is A.

In another aspect, the present application is directed to an IgG2 variant including an amino acid sequence having the formula:

ASTKGPSVFPLAPCSRSTSESTAALGCLVKDYFPEPVTVSWNSGALTSGVHTFPAVLQSSG
LYSLSSVVTVPSSNFGTQTYTCNVDHKPSNTKVDKTVERKCC-X(221)-X(222)-X(223)-
X(224)-X(225)-C-X(227)-X(228)-C-X(230)-X(231)-X(232)-X(233)-X(234)-X(235)-X(236)-
X(237)-X(238)-X(239)-X(240)-X(241)-L-X(243)-X(244)-X(245)-X(246)-X(247)-K-X(249)-
TLMIS-X(255)-TP-X(258)-V-X(260)-C-X(262)-X(263)-X(264)-X(265)-X(266)-X(267)-X(268)-
X(269)-X(270)-X(271)-X(272)-X(273)-X(274)-X(275)-X(276)-W-X(278)-V-X(280)-X(281)-
X(282)-X(283)-X(284)-X(285)-X(286)-A-X(288)-T-X(290)-X(291)-X(292)-X(293)-X(294)-
X(295)-X(296)-X(297)-X(298)-X(299)-X(300)-X(301)-X(302)-X(303)-X(304)-X(305)-
LTVVHQD-X(313)-LNG-X(317)-X(318)-Y-X(320)-C-X(322)-X(323)-X(324)-X(325)-X(326)-
X(327)-X(328)-X(329)-X(330)-X(331)-X(332)-X(333)-X(334)-X(335)-X(336)-X(337)-
KTKGQPREPQVYTLPPSREEMTKNQVSLTCLVKGFYPSDIAVEWESNGQPENNYKTTPPM
LDSDGSFFLYSKLTVDKSRWQQGNVFSCSVMHEALHNHYTQKSLSLSPGK-,

wherein
-X(221)- is selected from the group consisting of no amino acid, K and Y;
-X(222)- is selected from the group consisting of V, E and Y;
-X(223)- is selected from the group consisting of no amino acid, E and K;
-X(224)- is selected from the group consisting of E and Y;
-X(225)- is selected from the group consisting of no amino acid, E, K and W;
-X(227)- is selected from the group consisting of P, E, G, K and Y;
-X(228)- is selected from the group consisting of P, E, G, K and Y;
-X(230)- is selected from the group consisting of P, A, E, G and Y;
-X(231)- is selected from the group consisting of A, E, G, K, P and Y;
-X(232)- is selected from the group consisting of P, E, G, K and Y;
-X(233)- is selected from the group consisting of P, A, D, F, G, H, I, K, L, M, N, Q, R, S, T, V, W and Y;
-X(234)- is selected from the group consisting of V, D, E, F, G, H, I, K, M, N, P, Q, R, S, T, W and Y;
-X(235)- is selected from the group consisting of A, D, F, G, H, I, K, M, N, P, Q, R, S, T, V, W and Y;
-X(236)- is selected from the group consisting of no amino acid, A, D, E, F, H, I, K, L, M, N, P, Q, R, S, T, V, W and Y;
-X(237)- is selected from the group consisting of G, D, E, F, H, I, K, L, M, N, P, Q, R, S, T, V, W and Y;
-X(238)- is selected from the group consisting of P, D, E, F, G, H, I, K, L, M, N, Q, R, S, T, V, W and Y;
-X(239)- is selected from the group consisting of S, D, E, F, G, H, I, K, L, M, N, P, Q, R, T, V, W and Y;
-X(240)- is selected from the group consisting of V, A, I, M and T;
-X(241)- is selected from the group consisting of F, D, E, L, R, S, W and Y;
-X(243)- is selected from the group consisting of F, E, H, L, Q, R, W and Y;
-X(244)- is selected from the group consisting of P and H;
-X(245)- is selected from the group consisting of P and A;
-X(246)- is selected from the group consisting of K, D, E, H and Y;
-X(247)- is selected from the group consisting of P, G and V;
-X(249)- is selected from the group consisting of D, H, Q and Y;
-X(255)- is selected from the group consisting of R, E and Y;
-X(258)- is selected from the group consisting of E, H, S and Y;
-X(260)- is selected from the group consisting of T, D, E, H and Y;
-X(262)- is selected from the group consisting of V, A, E, F, I and T;
-X(263)- is selected from the group consisting of V, A, I, M and T;
-X(264)- is selected from the group consisting of V, A, D, E, F, G, H, I, K, L, M, N, P, Q, R, S, T, W and Y;
-X(265)- is selected from the group consisting of D, F, G, H, I, K, L, M, P, Q, R, S, T, V, W and Y;
-X(266)- is selected from the group consisting of V, A, I, M and T;
-X(267)- is selected from the group consisting of S, D, E, F, H, I, K, L, M, N, P, Q, R, V, W and Y;
-X(268)- is selected from the group consisting of H, D, E, F, G, I, K, L, M, P, R, T, V and W;
-X(269)- is selected from the group consisting of E, F, G, H, I, K, L, M, N, P, R, S, T, V, W and Y;
-X(270)- is selected from the group consisting of D, F, G, H, I, L, M, P, Q, R, S, T, W and Y;
-X(271)- is selected from the group consisting of P, A, D, E, F, G, H, I, K, L, M, N, Q, R, S, T, V, W and Y;
-X(272)- is selected from the group consisting of E, D, F, G, H, I, K, L, M, P, R, S, T, V, W and Y;
-X(273)- is selected from the group consisting of V and I;
-X(274)- is selected from the group consisting of Q, D, E, F, G, H, I, L, M, N, P, R, T, V, W and Y;
-X(275)- is selected from the group consisting of F, L and W;
-X(276)- is selected from the group consisting of N, D, E, F, G, H, I, L, M, P, R, S, T, V, W and Y;
-X(278)- is selected from the group consisting of Y, D, E, G, H, I, K, L, M, N, P, Q, R, S, T, V and W;
-X(280)- is selected from the group consisting of D, G, K, L, P and W;
-X(281)- is selected from the group consisting of G, D, E, K, N, P, Q and Y;
-X(282)- is selected from the group consisting of V, E, G, K, P and Y;
-X(283)- is selected from the group consisting of E, G, H, K, L, P, R and Y;
-X(284)- is selected from the group consisting of V, D, E, L, N, Q, T and Y;
-X(285)- is selected from the group consisting of H, D, E, K, Q, W and Y;
-X(286)- is selected from the group consisting of N, E, G, P and Y;
-X(288)- is selected from the group consisting of K, D, E and Y;
-X(290)- is selected from the group consisting of K, D, H, L, N and W;
-X(291)- is selected from the group consisting of P, D, E, G, H, I, Q and T;
-X(292)- is selected from the group consisting of R, D, E, T and Y;
-X(293)- is selected from the group consisting of E, F, G, H, I, L, M, N, P, R, S, T, V, W and Y;
-X(294)- is selected from the group consisting of E, F, G, H, I, K, L, M, P, R, S, T, V, W and Y;
-X(295)- is selected from the group consisting of Q, D, E, F, G, H, I, M, N, P, R, S, T, V, W and Y;
-X(296)- is selected from the group consisting of F, A, D, E, G, I, K, L, M, N, Q, R, S, T and V;
-X(297)- is selected from the group consisting of N, D, E, F, G, H, I, K, L, M, P, Q, R, S, T, V, W and Y;
-X(298)- is selected from the group consisting of S, E, F, H, I, K, M, Q, R, W and Y;
-X(299)- is selected from the group consisting of T, A, D, E, F, G, H, I, K, L, M, N, P, Q, R, S, V, W and Y;
-X(300)- is selected from the group consisting of F, A, D, E, G, H, K, M, N, P, Q, R, S, T, V and W;
-X(301)- is selected from the group consisting of R, D, E, H and Y;
-X(302)- is selected from the group consisting of V and I;
-X(303)- is selected from the group consisting of V, D, E and Y;
-X(304)- is selected from the group consisting of S, D, H, L, N and T;
-X(305)- is selected from the group consisting of V, E, T and Y;
-X(313)- is selected from the group consisting of W and F;
-X(317)- is selected from the group consisting of K, E and Q;
-X(318)- is selected from the group consisting of E, H, L, Q, R and Y;
-X(320)- is selected from the group consisting of K, D, F, G, H, I, L, N, P, S, T, V, W and Y;
-X(322)- is selected from the group consisting of K, D, F, G, H, I, P, S, T, V, W and Y;
-X(323)- is selected from the group consisting of V and I;
-X(324)- is selected from the group consisting of S, D, F, G, H, I, L, M, P, R, T, V, W and Y;
-X(325)- is selected from the group consisting of N, A, D, E, F, G, H, I, K, L, M, P, Q, R, S, T, V, W and Y;
-X(326)- is selected from the group consisting of K, I, L, P and T;
-X(327)- is selected from the group consisting of G, D, E, F, H, I, K, L, M, N, P, R, T, V, W and Y;
-X(328)- is selected from the group consisting of L, A, D, E, F, G, H, I, K, M, N, P, Q, R, S, T, V, W and Y;
-X(329)- is selected from the group consisting of P, D, E, F, G, H, I, K, L, M, N, Q, R, S, T, V, W and Y;
-X(330)- is selected from the group consisting of A, E, F, G, H, I, L, M, N, P, R, T, V, W and Y;
-X(331)- is selected from the group consisting of P, D, F, H, I, L, M, Q, R, T, V, W and Y;
-X(332)- is selected from the group consisting of I, A, D, E, F, H, K, L, M, N, P, Q, R, S, T, V, W and Y;
-X(333)- is selected from the group consisting of E, F, H, I, L, M, P, T and Y;
-X(334)- is selected from the group consisting of K, F, I, P and T;
-X(335)- is selected from the group consisting of T, D, F, G, H, I, L, M, N, P, R, S, V, W and Y;
-X(336)- is selected from the group consisting of I, E, K and Y; and
-X(337)- is selected from the group consisting of S, E, H and N.

In certain variations, the variant differs from SEQ ID NO:11 by at least one amino acid.

In a further aspect, the present application is directed to an IgG2 variant including an amino acid sequence having the formula:

ASTKGPSVFPLAPCSRSTSESTAALGCLVKDYFPEPVTVSWNSGALTSGVHTFPAVLQSSG
LYSLSSVVTVPSSNFGTQTYTCNVDHKPSNTKVDKTVERKCC-X(221)-V-E-C-X(227)-
PCPAPP-X(234)-X(235)-X(236)-X(237)-P-X(239)-X(240)-FLFPP-X(246)-PKDTLMIS-X(255)-
TP-X(258)-V-X(260)-CVV-X(264)-DV-X(267)-X(268)-ED-X(271)-X(272)-V-X(274)-FNW-
X(278)-VD-X(281)-V-X(283)-X(284)-HNAKT-X(290)-PR-X(293)-E-X(295)-FNSTFRVV-
X(304)-VLTVVHQDWLNGKEYKCKV-X(324)-N-X(326)-X(327)-X(328)-P-X(330)-P-X(332)-
X(333)-X(334)-
TISKTKGQPREPQVYTLPPSREEMTKNQVSLTCLVKGFYPSDIAVEWESNGQPENNYKTTP
PMLDSDGSFFLYSKLTVDKSRWQQGNVFSCSVMHEALHNHYTQKSLSLSPGK,

wherein
-X(221)- is selected from the group consisting of no amino acid and K;
-X(227)- is selected from the group consisting of P and G;
-X(234)- is selected from the group consisting of V, Y and I;
-X(235)- is selected from the group consisting of A, Y, I and D;
-X(236)- is selected from the group consisting of no amino acid, S and A;
-X(237)- is selected from the group consisting of G and D;
-X(239)- is selected from the group consisting of S, D, E, N, Q and T;
-X(240)- is selected from the group consisting of V, I and M;
-X(246)- is selected from the group consisting of K, H and Y;
-X(255)- is selected from the group consisting of R and Y;
-X(258)- is selected from the group consisting of E, H and Y;
-X(260)- is selected from the group consisting of T and H;
-X(264)- is selected from the group consisting of V, I, T and Y;
-X(267)- is selected from the group consisting of S, D and E;
-X(268)- is selected from the group consisting of H, D and E;
-X(271)- is selected from the group consisting of P and G;
-X(272)- is selected from the group consisting of E, Y, H, R and I;
-X(274)- is selected from the group consisting of Q and E;
-X(278)- is selected from the group consisting of Y and T;
-X(281)- is selected from the group consisting of G, D and E;
-X(283)- is selected from the group consisting of E, L and H;
-X(284)- is selected from the group consisting of V, E and D;
-X(290)- is selected from the group consisting of K and N;
-X(293)- is selected from the group consisting of E and R;
-X(295)- is selected from the group consisting of Q and E;
-X(304)- is selected from the group consisting of S and T;
-X(324)- is selected from the group consisting of S, G and I;
-X(326)- is selected from the group consisting of K and T;
-X(327)- is selected from the group consisting of G and D;
-X(328)- is selected from the group consisting of L, A, F, I and T;
-X(330)- is selected from the group consisting of A, L, Y and I;
-X(332)- is selected from the group consisting of I, D, E, N, Q and T;
-X(333)- is selected from the group consisting of E and Y; and
-X(334)- is selected from the group consisting of K, F, I and T;

In certain aspects, the variant differs from SEQ ID NO:2 by at least one amino acid.

In another aspect, the present application is directed to an IgG3 variant including two or more amino acid modifications as compared to SEQ ID NO:12. The modifications are selected from among C131S, R133K, G137E, G138S, S192N, L193F, Q196K, T199I, N203D, R214K R214T, L217P, L217R, L217S, T219S, T219C, T219Y, P220C P220G, L221D, L221-, deletion of the sequence LGD beginning at L221, T222K, T222V, deletion of T222, deletion of T223, H224E, H224P, deletion of T225, T225P, R228P, R228S, deletion of RCPEPKSCDTPPPCPRCPEPKSCDTPPPCPRCPEPKSCDTPPPCPR (SEQ ID NO:20) beginning at 228, E233P, L234V, L234F, L235A, deletion of G236, H268Q, Q274K, K276N, Y296F, F300Y, L309V, A327G, A330S, P331S, T339A, R355Q, E356D, M358L, S384N, N392K, M397V, K409R, Q419E, I422V, R435H, F436Y, and P445L. In certain embodiments, at least two of the amino acid modifications are in different domains. In various embodiments, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the modifications are in different domains.

In another embodiment, the an IgG3 variant includes an amino acid sequence having the formula:

ASTKGPSVFPLAP-X(131)-S-X(133)-
STSGGTAALGCLVKDYFPEPVTVSWNSGALTSGVHTFPAVLQSSGLYSLSSVVTVPSSSLG
TQTY-X(199)-CNVNHKPSNTKVDK-X(214)-VE-X(217)-K-X(219)-X(220)-X(221)-GD-X(222)-
THTCP-X(228)-
CPEPKSCDTPPPCPRCPEPKSCDTPPPCPRCPEPKSCDTPPPCPRCPAPELLGGPSVFLF
PPKPKDTLMISRTPEVTCVVVDVSHEDPEV-X(274)-F-X(276)-
WYVDGVEVHNAKTKPREEQYNST-X(300)-
RVVSVLTVLHQDWLNGKEYKCKVSNKALPAPIEKTISK-X(339)-KGQPREPQVYTLPPSR-
X(356)-E-X(358)-TKNQVSLTCLVKGFYPSDIAVEWES-X(384)-GQPENNY-X(392)-TTPP-
X(397)-LDSDGSFFLYSKLTVDKSRWQQGN-X(422)-FSCSVMHEALHN-X(435)-X(436)-
TQKSLSLSPGK

wherein
X(131) is selected from the group consisting of C and S;
X(133) is selected from the group consisting of R and K;
X(199) is selected from the group consisting of T and I;
X(214) is selected from the group consisting of R and K;
X(217) is selected from the group consisting of L and P;
X(219) is selected from the group consisting of T and S;
X(220) is selected from the group consisting of P and C;
X(221) is selected from the group consisting of D L, and the sequence LGD;
X(222) is selected from the group consisting of T and K;
X(228) is selected from the group consisting of R, the sequence RCPEPKSCDTPPPCPRCPEPKSCDTPPPCPRCPEPKSCDTPPPCPR (SEQ ID NO:20) and P;
X(274) is selected from the group consisting of Q and K;
X(276) is selected from the group consisting of K and N;
X(300) is selected from the group consisting of F and Y;
X(339) is selected from the group consisting of T and A;
X(356) is selected from the group consisting of E and D;
X(358) is selected from the group consisting of M and L;
X(384) is selected from the group consisting of S and N;
X(392) is selected from the group consisting of N and K;
X(397) is selected from the group consisting of M and V;
X(422) is selected from the group consisting of I and V;
X(435) is selected from the group consisting of R and H; and
X(436) is selected from the group consisting of F and Y.

In certain variations, the formula has at least two amino acid modifications as compared to SEQ ID NO:12. In further variations, the two of modifications can in different domains. In various embodiments, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the modifications are in different domains.

In another aspect, the present application is directed to an IgG3 variant including two or more amino acid modifications as compared to SEQ ID NO:12. The modifications can be selected from among C131S, R133K, G137E, G138S, S192N, L193F, Q196K, T199I, N203D, R214K R214T, L217P, L217R, L217S, T219S, T219C, T219Y, P220C P220G, L221D, the deletion of L221, deletion of GD, T222K, T222V, the deletion of T222, the deletion of T223, H224E, H224P, the deletion of T225, T225P, R228P, R228S, deletion of RCPEPKSCDTPPPCPRCPEPKSCDTPPPCPRCPEPKSCDTPPPCPR (SEQ ID NO:20) beginning at R228, E233P, L234V, L234F, L235A, G236-, H268Q, Q274K, K276N, Y296F, F300Y, L309V, A327G, A330S, P331S, T339A, R355Q, E356D, M358L, S384N, N392K, M397V, K409R, Q419E, I422V, R435H, F436Y, and P445L. In certain embodiments, at least two of the amino acid modifications are in different domains. In various embodiments, the formula has at least 1, 2, 3, 4, 5, 6, 7, 8, 9, 10 or more amino acid modifications as compared to an amino acid sequence including SEQ ID NO:11. In additional embodiments, at least 2, 3, or 4 of the modifications are in different domains.

In another aspect, the present application is directed to an IgG3 variant including an amino acid sequence having the formula:

-ASTKGPSVFPLAP-X(131)-S-X(133)-STS-X(137)-X(138)-
TAALGCLVKDYFPEPVTVSWNSGALTSGVHTFPAVLQSSGLYSLSSVVTVPSS-X(192)-
X(193)-GT-X(196)-TY-X(199)-CNV-X(203)-HKPSNTKVDK-X(214)-VE-X(217)-K-X(219)-
X(220)-X(221)-X(222)-X(223)-X(224)-X(225)-C-X(227)-X(228)-C-X(230)-X(231)-X(232)-
X(233)-X(234)-X(235)-X(236)-X(237)-X(238)-X(239)-X(240)-X(241)-L-X(243)-X(244)-X(245)-
X(246)-X(247)-K-X(249)-TLMIS-X(255)-TP-X(258)-V-X(260)-C-X(262)-X(263)-X(264)-
X(265)-X(266)-X(267)-X(268)-X(269)-X(270)-X(271)-X(272)-X(273)-X(274)-X(275)-X(276)-
W-X(278)-V-X(280)-X(281)-X(282)-X(283)-X(284)-X(285)-X(286)-A-X(288)-T-X(290)-X(291)-
X(292)-X(293)-X(294)-X(295)-X(296)-X(297)-X(298)-X(299)-X(300)-X(301)-X(302)-X(303)-
X(304)-X(305)-LTV-X(309)-HQD-X(313)-LNG-X(317)-X(318)-Y-X(320)-C-X(322)-X(323)-
X(324)-X(325)-X(326)-X(327)-X(328)-X(329)-X(330)-X(331)-X(332)-X(333)-X(334)-X(335)-
X(336)-X(337)-K-X(339)-KGQPREPQVYTLPPS-X(355)-X(356)-E-X(358)-
TKNQVSLTCLVKGFYPSDIAVEWES-X(384)-GQPENNY-X(392)-TTPP-X(397)-
LDSDGSFFLYS-X(409)-LTVDKSRWQ-X(419)-GN-X(422)-FSCSVMHEALHN-X(435)-
X(436)-TQKSLSLS-X(445)-GK

wherein
-X(131)- is selected from the group consisting of C and S;
-X(133)- is selected from the group consisting of R and K;
-X(137)- is selected from the group consisting of E and G;
-X(138)- is selected from the group consisting of S and G;
-X(192)- is selected from the group consisting of N and S;
-X(193)- is selected from the group consisting of F and L;
-X(196)- is selected from the group consisting of Q and K;
-X(199)- is selected from the group consisting of T and I;
-X(203)- is selected from the group consisting of D and N;
-X(214)- is selected from the group consisting of T, K and R;
-X(217)- is selected from the group consisting of R, P, L and S;
-X(219)- is selected from the group consisting of C, S, T and Y;
-X(220)- is selected from the group consisting of C, P and G;
-X(221)- is selected from the group consisting of no amino acid, D, K, Y, L, and the sequence LGD;
-X(222)- is selected from the group consisting of V, K, T, no amino acid, E and Y;
-X(223)- is selected from the group consisting of no amino acid, T, E and K;
-X(224)- is selected from the group consisting of E, H, P and Y;
-X(225)- is selected from the group consisting of no amino acid, T, P, E, K and W;
-X(227)- is selected from the group consisting of P, E, G, K and Y;
-X(228)- is selected from the group consisting of P, S, E, G, K, Y, R, and the sequence

(SEQ ID NO: 20)

RCPEPKSCDTPPPCPRCPEPKSCDTPPPCPRCPEPKSCDTPPPCPR;

-X(230)- is selected from the group consisting of P, A, E, G and Y;
-X(231)- is selected from the group consisting of A, E, G, K, P and Y;
-X(232)- is selected from the group consisting of P, E, G, K and Y;
-X(233)- is selected from the group consisting of P, E, A, D, F, G, H, I, K, L, M, N, Q, R, S, T, V, W and Y;
-X(234)- is selected from the group consisting of V, L, F, D, E, F, G, H, I, K, M, N, P, Q, R, S, T, W and Y;
-X(235)- is selected from the group consisting of A, L, D, F, G, H, I, K, M, N, P, Q, R, S, T, V, W, and Y;
-X(236)- is selected from the group consisting of no amino acid, G, A, D, E, F, H, I, K, L, M, N, P, Q, R, S, T, V, W and Y;
-X(237)- is selected from the group consisting of G, D, E, F, H, I, K, L, M, N, P, Q, R, S, T, V, W and Y;
-X(238)- is selected from the group consisting of P, D, E, F, G, H, I, K, L, M, N, Q, R, S, T, V, W and Y;
-X(239)- is selected from the group consisting of S, D, E, F, G, H, I, K, L, M, N, P, Q, R, T, V, W and Y;
-X(240)- is selected from the group consisting of V, A, I, M and T;
-X(241)- is selected from the group consisting of F, D, E, L, R, S, W and Y;
-X(243)- is selected from the group consisting of F, E, H, L, Q, R, and Y;
-X(244)- is selected from the group consisting of P and H;
-X(245)- is selected from the group consisting of P and A;
-X(246)- is selected from the group consisting of, K, D, E, H and Y;
-X(247)- is selected from the group consisting of P, G and V;
-X(249)- is selected from the group consisting of D, H, Q and Y;
-X(255)- is selected from the group consisting of R and Y;
-X(258)- is selected from the group consisting of E, H, S and Y;
-X(260)- is selected from the group consisting of T, D, E, H and Y;
-X(262)- is selected from the group consisting of V, A, E, F, I and T;
-X(263)- is selected from the group consisting of V, A, I, M and T;
-X(264)- is selected from the group consisting of V, A, D, E, F, G, H, I, K, L, M, N, P, Q, R, S, T, W, and Y;
-X(265)- is selected from the group consisting of D, F, G, H, I, K, L, M, P, Q, R, S, T, V, W and Y;
-X(266)- is selected from the group consisting of V, A, I, M and T;
-X(267)- is selected from the group consisting of S, D, E, F, H, I, K, L, M, N, P, Q, R, V, W and Y;
-X(268)- is selected from the group consisting of H, Q, D, E, F, G, I, K, L, M, P, R, T, V and W;
-X(269)- is selected from the group consisting of E, F, G, H, I, K, L, M, N, P, R, S, T, V, W and Y;
-X(270)- is selected from the group consisting of D, F, G, H, I, L, M, P, Q, R, S, T, W and Y;
-X(271)- is selected from the group consisting of P, A, D, E, F, G, H, I, K, L, M, N, Q, R, S, T, V, W and Y;
-X(272)- is selected from the group consisting of E, D, F, G, H, I, K, L, M, P, R, S, T, V, W and Y;
-X(273)- is selected from the group consisting of V and I;
-X(274)- is selected from the group consisting of Q, K, D, E, F, G, H, I, L, M, N, P, R, T, V, W and Y;
-X(275)- is selected from the group consisting of FL and W;
-X(276)- is selected from the group consisting of N, K, D, E, F, G, H, I, L, M, P, R, S, T, V, W and Y;
-X(278)- is selected from the group consisting of Y, D, E, G, H, I, K, L, M, N, P, Q, R, S, T, V and W;
-X(280)- is selected from the group consisting of D, G, K, L, P and W;
-X(281)- is selected from the group consisting of G, D, E, K, N, P, Q and Y;
-X(282)- is selected from the group consisting of V, E, G, K, P and Y;
-X(283)- is selected from the group consisting of E, G, H, K, L, P, R and Y;
-X(284)- is selected from the group consisting of V, D, E, L, N, Q, T and Y;
-X(285)- is selected from the group consisting of H, D, E, K, Q, W and Y;
-X(286)- is selected from the group consisting of N, E, G, P and Y;
-X(288)- is selected from the group consisting of K, D, E and Y;
-X(290)- is selected from the group consisting of K, D, H, L, N and W;
-X(291)- is selected from the group consisting of P, D, E, G, H, I, Q and T;
-X(292)- is selected from the group consisting of R, D, E, T and Y;
-X(293)- is selected from the group consisting of E, F, G, H, I, L, M, N, P, R, S, T, V, W and Y;
-X(294)- is selected from the group consisting of E, F, G, H, I, K, L, M, P, R, S, T, V, W and Y;
-X(295)- is selected from the group consisting of Q, D, E, F, G, H, I, M, N, P, R, S, T, V, W and Y;
-X(296)- is selected from the group consisting of F, Y, A, D, E, G, I, K, L, M, N, Q, R, S, T and V;
-X(297)- is selected from the group consisting of N, D, E, F, G, H, I, K, L, M, P, Q, R, S, T, V, W and Y;
-X(298)- is selected from the group consisting of S, E, F, H, I, K, M, Q, R, W and Y;
-X(299)- is selected from the group consisting of T, A, D, E, F, G, H, I, K, L, M, N, P, Q, R, S, V, W and Y;
-X(300)- is selected from the group consisting of F, Y, A, D, E, G, H, K, M, N, P, Q, R, S, T, V and W;
-X(301)- is selected from the group consisting of R, D, E, H and Y;
-X(302)- is selected from the group consisting of V and I;
-X(303)- is selected from the group consisting of V, D, E and Y;
-X(304)- is selected from the group consisting of S, D, H, L, N and T;
-X(305)- is selected from the group consisting of V, E, T and Y;
-X(309)- is selected from the group consisting of V and L;
-X(313)- is selected from the group consisting of W and F;
-X(317)- is selected from the group consisting of K, E and Q;
-X(318)- is selected from the group consisting of E, H, L, Q, R and Y;
-X(320)- is selected from the group consisting of K, D, F, G, H, I, L, N, P, S, T, V, W and Y;
-X(322)- is selected from the group consisting of K, D, F, G, H, I, P, S, T, V, W and Y;
-X(323)- is selected from the group consisting of V and I;
-X(324)- is selected from the group consisting of S, D, F, G, H, I, L, M, P, R, T, V, W and Y;
-X(325)- is selected from the group consisting of N, A, D, E, F, G, H, I, K, L, M, P, Q, R, S, T, V, W and Y;
-X(326)- is selected from the group consisting of K, I, L, P and T;
-X(327)- is selected from the group consisting of G, A, D, E, F, H, I, K, L, M, N, P, R, T, V, W and Y;
-X(328)- is selected from the group consisting of L, A, D, E, F, G, H, I, K, M, N, P, Q, R, S, T, V, W and Y;
-X(329)- is selected from the group consisting of P, D, E, F, G, H, I, K, L, M, N, Q, R, S, T, V, W and Y;
-X(330)- is selected from the group consisting of A, S, E, F, G, H, I, L, M, N, P, R, T, V, W and Y;
-X(331)- is selected from the group consisting of P, S, D, F, H, I, L, M, Q, R, T, V, W and Y;
-X(332)- is selected from the group consisting of I, A, D, E, F, H, K, L, M, N, P, Q, R, S, T, V, W and Y;
-X(333)- is selected from the group consisting of E, F, H, I, L, M, P, T and Y;
-X(334)- is selected from the group consisting of K, F, I, P and T;
-X(335)- is selected from the group consisting of T, D, F, G, H, I, L, M, N, P, R, S, V, W and Y;
-X(336)- is selected from the group consisting of I, E, K and Y;
-X(337)- is selected from the group consisting of S, E, H and N;
-X(339)- is selected from the group consisting of T and A;
-X(355)- is selected from the group consisting of R and Q;
-X(356)- is selected from the group consisting of E and D;
-X(358)- is selected from the group consisting of M and L;
-X(384)- is selected from the group consisting of N and S;
-X(392)- is selected from the group consisting of K and N;
-X(397)- is selected from the group consisting of M and V;
-X(409)- is selected from the group consisting of K and R;
-X(419)- is selected from the group consisting of Q and E;
-X(422)- is selected from the group consisting of V and I;
-X(435)- is selected from the group consisting of H and R;
-X(436)- is selected from the group consisting of Y and F; and
-X(445)- is selected from the group consisting of P and L.

In one variation, a first modification can be selected from among C131S, R133K, G137E, G138S, S192N, L193F, Q196K, T199I, N203D, R214K R214T, L217P, L217R, L217S, T219S, T219C, T219Y, P220C P220G, L221D, deletion of L221, deletion of the sequence LGD beginning at L221, T222K, T222V, deletion of T222, deletion of T223, H224E, H224P, deletion of T225, T225P, R228P, R228S, deletion of the sequence RCPEPKSCDTPPPCPRCPEPKSCDTPPPCPRCPEPKSCDTPPPCPR (SEQ ID NO:20) beginning at 228, E233P, L234V, L234F, L235A, deletion of G236, H268Q, Q274K, K276N, Y296F, F300Y, L309V, A327G, A330S, P331S, T339A, R355Q, E356D, M358L, S384N, N392K, M397V, K409R, Q419E, 1422V, R435H, F436Y, and P445L. In a further variation, a second modification is selected from among 221K, 221Y, 222E, 222Y, 223E, 223K, 224Y, 225E, 225K, 225W, 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, 337N. In various embodiments, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the modifications are in different domains.

In a further aspect, the present application is directed to an IgG3 variant amino acid sequence having at least two amino acid modifications as compared to SEQ ID NO:13, wherein a first modification is selected from among C131S, R133K, G137E, G138S, S192N, L193F, Q196K, T199I, N203D, R214K R214T, L217P, L217R, L217S, T219S, T219C, T219Y, P220C P220G, L221D, deletion of L221, deletion of the sequence LGD beginning at L221, T222K, T222V, deletion of T222, deletion of T223, H224E, H224P, deletion of T225, T225P, R228P, R228S, deletion of the sequence RCPEPKSCDTPPPCPRCPEPKSCDTPPPCPRCPEPKSCDTPPPCPR (SEQ ID NO: 20) beginning at 228, E233P, L234V, L234F, L235A, deletion of G236, H268Q, Q274K, K276N, Y296F, F300Y, L309V, A327G, A330S, P331S, T339A, R355Q, E356D, M358L, S384N, N392K, M397V, K409R, Q419E, I422V, R435H, F436Y, and P445L, and a second modification is selected from among 221K, 221Y, 222E, 222Y, 223E, 223K, 224Y, 225E, 225K, 225W, 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N. In various embodiments, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the modifications are in different domains.

In another aspect, the present application is directed to an IgG3 variant including an amino acid sequence having the formula:

ASTKGPSVFPLAP-X(131)-S-X(133)-STS-X(137)-X(138)-
TAALGCLVKDYFPEPVTVSWNSGALTSGVHTFPAVLQSSGLYSLSSVVTVPSS-X(192)-
X(193)-GT-X(196)-TY-X(199)-CNV-X(203)-HKPSNTKVDK-X(214)-VE-X(217)-K-X(219)-
X(220)-X(221)-X(222)-X(223)-X(224)-X(225)-C-X(227)-X(228)-CPAP-X(233)-X(234)-X(235)-
X(236)-X(237)-P-X(239)-X(240)-FLFPP-X(246)-PKDTLMIS-X(255)-TP-X(258)-V-X(260)-
CVV-X(264)-DV-X(267)-X(268)-ED-X(271)-X(272)-V-X(274)-F-X(276)-W-X(278)-VD-X(281)-
V-X(283)-X(284)-HNAKT-X(290)-PR-X(293)-E-X(295)-X(296)-NST-X(300)-RVV-X(304)-
VLTV-X(309)-HQDWLNGKEYKCKV-X(324)-N-X(326)-X(327)-X(328)-P-X(330)-X(331)-
X(332)-X(333)-X(334)-TISK-X(339)-KGQPREPQVYTLPPS-X(355)-X(356)-E-X(358)-
TKNQVSLTCLVKGFYPSDIAVEWES-X(384)-GQPENNY-X(392)-TTPP-X(397)-
LDSDGSFFLYS-X(409)-LTVDKSRWQ-X(419)-GN-X(422)-FSCSVMHEALHN-X(435)-
X(436)-TQKSLSLS-X(445)-GK;

wherein
-X(131)- is selected from the group consisting of C and S;
-X(133)- is selected from the group consisting of R and K;
-X(137)- is selected from the group consisting of E and G;
-X(138)- is selected from the group consisting of S and G;
-X(192)- is selected from the group consisting of N and S;
-X(193)- is selected from the group consisting of F and L;
-X(196)- is selected from the group consisting of Q and K;
-X(199)- is selected from the group consisting of T and I;
-X(203)- is selected from the group consisting of D and N;
-X(214)- is selected from the group consisting of T, K and R;
-X(217)- is selected from the group consisting of R, P, L and S;
-X(219)- is selected from the group consisting of C, S, T and Y;
-X(220)- is selected from the group consisting of C, P and G;
-X(221)- is selected from the group consisting of no amino acid, D, L, K, and the sequence LGD;
-X(222)- is selected from the group consisting of V, K, T, and no amino acid;
-X(223)- is selected from the group consisting of no amino acid and T;
-X(224)- is selected from the group consisting of E, H and P;
-X(225)- is selected from the group consisting of no amino acid, T and P;
-X(227)- is selected from the group consisting of P and G;
-X(228)- is selected from the group consisting of P, R, S, and the sequence

(SEQ ID NO: 20)

RCPEPKSCDTPPPCPRCPEPKSCDTPPPCPRCPEPKSCDTPPPCPR;

-X(233)- is selected from the group consisting of P and E;
-X(234)- is selected from the group consisting of V, LF, Y and I;
-X(235)- is selected from the group consisting of A, L, Y, I and D;
-X(236)- is selected from the group consisting of no amino acid, G, S and A;
-X(237)- is selected from the group consisting of G and D;
-X(239)- is selected from the group consisting of S, D, E, N, Q and T;
-X(240)- is selected from the group consisting of V, I and M;
-X(246)- is selected from the group consisting of K, H and Y;
-X(255)- is selected from the group consisting of R and Y;
-X(258)- is selected from the group consisting of E, H and Y;
-X(260)- is selected from the group consisting of T and H;
-X(264)- is selected from the group consisting of V, I, T and Y;
-X(267)- is selected from the group consisting of S, D and E;
-X(268)- is selected from the group consisting of H, Q, D and E;
-X(271)- is selected from the group consisting of P and G;
-X(272)- is selected from the group consisting of E, Y, H, R and I;
-X(274)- is selected from the group consisting of Q, K and E;
-X(276)- is selected from the group consisting of N and K;
-X(278)- is selected from the group consisting of Y and T;
-X(281)- is selected from the group consisting of G, D and E;
-X(283)- is selected from the group consisting of E, L and H;
-X(284)- is selected from the group consisting of V, E and D;
-X(290)- is selected from the group consisting of K and N;
-X(293)- is selected from the group consisting of E and R;
-X(295)- is selected from the group consisting of Q and E;
-X(296)- is selected from the group consisting of F and Y;
-X(300)- is selected from the group consisting of F and Y;
-X(304)- is selected from the group consisting of S and T;
-X(309)- is selected from the group consisting of V and L;
-X(324)- is selected from the group consisting of S, G and I;
-X(326)- is selected from the group consisting of K and T;
-X(327)- is selected from the group consisting of G, A and D;
-X(328)- is selected from the group consisting of L, A, F, I and T;
-X(330)- is selected from the group consisting of A, S, L, Y and I;
-X(331)- is selected from the group consisting of P and S;
-X(332)- is selected from the group consisting of I, D, E, N, Q and T;
-X(333)- is selected from the group consisting of E and Y;
-X(334)- is selected from the group consisting of K, F, I and T;
-X(339)- is selected from the group consisting of T and A;
-X(355)- is selected from the group consisting of R and Q;
-X(356)- is selected from the group consisting of E and D;
-X(358)- is selected from the group consisting of M and L;
-X(384)- is selected from the group consisting of N and S;
-X(392)- is selected from the group consisting of K and N;
-X(397)- is selected from the group consisting of M and V;
-X(409)- is selected from the group consisting of K and R;
-X(419)- is selected from the group consisting of Q and E;
-X(422)- is selected from the group consisting of V and I;
-X(435)- is selected from the group consisting of H and R;
-X(436)- is selected from the group consisting of Y and F;
-X(445)- is selected from the group consisting of P and L.

In certain variations, a first modification is selected from among C131S, R133K, G137E, G138S, S192N, L193F, Q196K, T199I, N203D, R214K R214T, L217P, L217R, L217S, T219S, T219C, T219Y, P220C P220G, L221D, deletion of L221, deletion of the sequence LGD beginning at L221, T222K, T222V, deletion of T222, deletion of T223, H224E, H224P, deletion of T225, T225P, R228P, R228S, deletion of R, deletion of the sequence RCPEPKSCDTPPPCPRCPEPKSCDTPPPCPRCPEPKSCDTPPPCPR (SEQ ID NO:20) beginning at 228, E233P, L234V, L234F, L235A, deletion of G236, H268Q, Q274K, K276N, Y296F, F300Y, L309V, A327G, A330S, P331S, T339A, R355Q, E356D, M358L, S384N, N392K, M397V, K409R, Q419E, 1422V, R435H, F436Y, and P445L. In a further variation, a second modification is selected from among 221K, 227G, 234Y, 234I, 235Y, 235I, 235D, 236S, 236A, 237D, 239D, 239E, 239N, 239Q, 239T, 240I, 240M, 246H, 246Y, 255Y, 258H, 258Y, 260H, 264I, 264T, 264Y, 267D, 267E, 268D, 268E, 271G, 272Y, 272H, 272R, 272I, 274E, 278T, 281D, 281E, 283L, 283H, 284E, 284D, 290N, 293R, 295E, 304T, 324G, 324I, 326T, 327D, 328A, 328F, 328I, 328T, 330L, 330Y, 330I, 332D, 332E, 332N, 332Q, 332T, 333Y, 334F, 334I, and 334T. In various embodiments, the formula has at least 1, 2, 3, 4, 5, 6, 7, 8, 9, 10 or more first and/or second amino acid modifications as compared to an amino acid sequence including SEQ ID NO:12. In additional embodiments, at least 2, 3, or 4 of the modifications are in different domains.

In another aspect, the present application is directed to an IgG3 variant including an amino acid sequence having the formula:

C-X(227)-X(228)-C-X(230)-X(231)-X(232)-X(233)-X(234)-X(235)-X(236)-X(237)-X(238)-
X(239)-X(240)-X(241)-L-X(243)-X(244)-X(245)-X(246)-X(247)-K-X(249)-TLMIS-X(255)-TP-
X(258)-V-X(260)-C-X(262)-X(263)-X(264)-X(265)-X(266)-X(267)-X(268)-X(269)-X(270)-
X(271)-X(272)-X(273)-X(274)-X(275)-X(276)-W-X(278)-V-X(280)-X(281)-X(282)-X(283)-
X(284)-X(285)-X(286)-A-X(288)-T-X(290)-X(291)-X(292)-X(293)-X(294)-X(295)-X(296)-
X(297)-X(298)-X(299)-X(300)-X(301)-X(302)-X(303)-X(304)-X(305)-LTV-X(309)-HQD-
X(313)-LNG-X(317)-X(318)-Y-X(320)-C-X(322)-X(323)-X(324)-X(325)-X(326)-X(327)-
X(328)-X(329)-X(330)-X(331)-X(332)-X(333)-X(334)-X(335)-X(336)-X(337)-K-X(339)-
KGQPREPQVYTLPPS-X(355)-X(356)-E-X(358)-TKNQVSLTCLVKGFYPSDIAVEWES-
X(384)-GQPENNY-X(392)-TTPP-X(397)-LDSDGSFFLYS-X(409)-LTVDKSRWQ-X(419)-GN-
X(422)-FSCSVMHEALHN-X(435)-X(436)-TQKSLSLS-X(445)-GK

wherein
-X(227)- is selected from the group consisting of P, E, G, K and Y;
-X(228)- is selected from the group consisting of P, S, E, G, K, Y, R, and the sequence

(SEQ ID NO: 20)

RCPEPKSCDTPPPCPRCPEPKSCDTPPPCPRCPEPKSCDTPPPCPR;

-X(230)- is selected from the group consisting of P, A, E, G and Y;
-X(231)- is selected from the group consisting of A, E, G, K, P and Y;
-X(232)- is selected from the group consisting of P, E, G, K and Y;
-X(233)- is selected from the group consisting of P, E, A, D, F, G, H, I, K, L, M, N, Q, R, S, T, V, W and Y;
-X(234)- is selected from the group consisting of V, L, F, D, E, F, G, H, I, K, M, N, P, Q, R, S, T, W and Y;
-X(235)- is selected from the group consisting of A, L, D, F, G, H, I, K, M, N, P, Q, R, S, T, V, W, and Y;
-X(236)- is selected from the group consisting of no amino acid, G, A, D, E, F, H, I, K, L, M, N, P, Q, R, S, T, V, W and Y;
-X(237)- is selected from the group consisting of G, D, E, F, H, I, K, L, M, N, P, Q, R, S, T, V, W and Y;
-X(238)- is selected from the group consisting of P, D, E, F, G, H, I, K, L, M, N, Q, R, S, T, V, W and Y;
-X(239)- is selected from the group consisting of S, D, E, F, G, H, I, K, L, M, N, P, Q, R, T, V, W and Y;
-X(240)- is selected from the group consisting of V, A, I, M and T;
-X(241)- is selected from the group consisting of F, D, E, L, R, S, W and Y;
-X(243)- is selected from the group consisting of F, E, H, L, Q, R, and Y;
-X(244)- is selected from the group consisting of P and H;
-X(245)- is selected from the group consisting of P and A;
-X(246)- is selected from the group consisting of, K, D, E, H and Y;
-X(247)- is selected from the group consisting of P, G and V;
-X(249)- is selected from the group consisting of D, H, Q and Y;
-X(255)- is selected from the group consisting of R and Y;
-X(258)- is selected from the group consisting of E, H, S and Y;
-X(260)- is selected from the group consisting of T, D, E, H and Y;
-X(262)- is selected from the group consisting of V, A, E, F, I and T;
-X(263)- is selected from the group consisting of V, A, I, M and T;
-X(264)- is selected from the group consisting of V, A, D, E, F, G, H, I, K, L, M, N, P, Q, R, S, T, W, and Y;
-X(265)- is selected from the group consisting of D, F, G, H, I, K, L, M, P, Q, R, S, T, V, W and Y;
-X(266)- is selected from the group consisting of V, A, I, M and T;
-X(267)- is selected from the group consisting of S, D, E, F, H, I, K, L, M, N, P, Q, R, V, W and Y;
-X(268)- is selected from the group consisting of H, Q, D, E, F, G, I, K, L, M, P, R, T, V and W;
-X(269)- is selected from the group consisting of E, F, G, H, I, K, L, M, N, P, R, S, T, V, W and Y;
-X(270)- is selected from the group consisting of D, F, G, H, I, L, M, P, Q, R, S, T, W and Y;
-X(271)- is selected from the group consisting of P, A, D, E, F, G, H, I, K, L, M, N, Q, R, S, T, V, W and Y;
-X(272)- is selected from the group consisting of E, D, F, G, H, I, K, L, M, P, R, S, T, V, W and Y;
-X(273)- is selected from the group consisting of V and I;
-X(274)- is selected from the group consisting of Q, K, D, E, F, G, H, I, L, M, N, P, R, T, V, W and Y;
-X(275)- is selected from the group consisting of FL and W;
-X(276)- is selected from the group consisting of N, K, D, E, F, G, H, I, L, M, P, R, S, T, V, W and Y;
-X(278)- is selected from the group consisting of Y, D, E, G, H, I, K, L, M, N, P, Q, R, S, T, V and W;
-X(280)- is selected from the group consisting of D, G, K, L, P and W;
-X(281)- is selected from the group consisting of G, D, E, K, N, P, Q and Y;
-X(282)- is selected from the group consisting of V, E, G, K, P and Y;
-X(283)- is selected from the group consisting of E, G, H, K, L, P, R and Y;
-X(284)- is selected from the group consisting of V, D, E, L, N, Q, T and Y;
-X(285)- is selected from the group consisting of H, D, E, K, Q, W and Y;
-X(286)- is selected from the group consisting of N, E, G, P and Y;
-X(288)- is selected from the group consisting of K, D, E and Y;
-X(290)- is selected from the group consisting of K, D, H, L, N and W;
-X(291)- is selected from the group consisting of P, D, E, G, H, I, Q and T;
-X(292)- is selected from the group consisting of R, D, E, T and Y;
-X(293)- is selected from the group consisting of E, F, G, H, I, L, M, N, P, R, S, T, V, W and Y;
-X(294)- is selected from the group consisting of E, F, G, H, I, K, L, M, P, R, S, T, V, W and Y;
-X(295)- is selected from the group consisting of Q, D, E, F, G, H, I, M, N, P, R, S, T, V, W and Y;
-X(296)- is selected from the group consisting of F, Y, A, D, E, G, I, K, L, M, N, Q, R, S, T and V;
-X(297)- is selected from the group consisting of N, D, E, F, G, H, I, K, L, M, P, Q, R, S, T, V, W and Y;
-X(298)- is selected from the group consisting of S, E, F, H, I, K, M, Q, R, W and Y;
-X(299)- is selected from the group consisting of T, A, D, E, F, G, H, I, K, L, M, N, P, Q, R, S, V, W and Y;
-X(300)- is selected from the group consisting of F, Y, A, D, E, G, H, K, M, N, P, Q, R, S, T, V and W;
-X(301)- is selected from the group consisting of R, D, E, H and Y;
-X(302)- is selected from the group consisting of V and I;
-X(303)- is selected from the group consisting of V, D, E and Y;
-X(304)- is selected from the group consisting of S, D, H, L, N and T;
-X(305)- is selected from the group consisting of V, E, T and Y;
-X(309)- is selected from the group consisting of V and L;
-X(313)- is selected from the group consisting of W and F;
-X(317)- is selected from the group consisting of K, E and Q;
-X(318)- is selected from the group consisting of E, H, L, Q, R and Y;
-X(320)- is selected from the group consisting of K, D, F, G, H, I, L, N, P, S, T, V, W and Y;
-X(322)- is selected from the group consisting of K, D, F, G, H, I, P, S, T, V, W and Y;
-X(323)- is selected from the group consisting of V and I;
-X(324)- is selected from the group consisting of S, D, F, G, H, I, L, M, P, R, T, V, W and Y;
-X(325)- is selected from the group consisting of N, A, D, E, F, G, H, I, K, L, M, P, Q, R, S, T, V, W and Y;
-X(326)- is selected from the group consisting of K, I, L, P and T;
-X(327)- is selected from the group consisting of G, A, D, E, F, H, I, K, L, M, N, P, R, T, V, W and Y;
-X(328)- is selected from the group consisting of L, A, D, E, F, G, H, I, K, M, N, P, Q, R, S, T, V, W and Y;
-X(329)- is selected from the group consisting of P, D, E, F, G, H, I, K, L, M, N, Q, R, S, T, V, W and Y;
-X(330)- is selected from the group consisting of A, S, E, F, G, H, I, L, M, N, P, R, T, V, W and Y;
-X(331)- is selected from the group consisting of P, S, D, F, H, I, L, M, Q, R, T, V, W and Y;
-X(332)- is selected from the group consisting of I, A, D, E, F, H, K, L, M, N, P, Q, R, S, T, V, W and Y;
-X(333)- is selected from the group consisting of E, F, H, I, L, M, P, T and Y;
-X(334)- is selected from the group consisting of K, F, I, P and T;
-X(335)- is selected from the group consisting of T, D, F, G, H, I, L, M, N, P, R, S, V, W and Y;
-X(336)- is selected from the group consisting of I, E, K and Y;
-X(337)- is selected from the group consisting of S, E, H and N;
-X(339)- is selected from the group consisting of T and A;
-X(355)- is selected from the group consisting of R and Q;
-X(356)- is selected from the group consisting of E and D;
-X(358)- is selected from the group consisting of M and L;
-X(384)- is selected from the group consisting of N and S;
-X(392)- is selected from the group consisting of K and N;
-X(397)- is selected from the group consisting of M and V;
-X(409)- is selected from the group consisting of K and R;
-X(419)- is selected from the group consisting of Q and E;
-X(422)- is selected from the group consisting of V and 1;
-X(435)- is selected from the group consisting of H and R;
-X(436)- is selected from the group consisting of Y and F; and
-X(445)- is selected from the group consisting of P and L

In various embodiments, a first modification is selected from among R228P, R228S, deletion of the sequence RCPEPKSCDTPPPCPRCPEPKSCDTPPPCPRCPEPKSCDTPPPCPR (SEQ ID NO:20) beginning at 228, E233P, L234V, L234F, L235A, deletion of G236, H268Q, Q274K, K276N, Y296F, F300Y, L309V, A327G, A330S, P331S, T339A, R355Q, E356D, M358L, S384N, N392K, M397V, K409R, Q419E, 1422V, R435H, F436Y, and P445L, and/or a second modification is selected from among 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N. In various embodiments, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the first and/or second modifications are in different domains. Alternatively, the substitutions can be selected from those beginning at position 230.

In another aspect, the present application is directed to an IgG3 variant amino acid sequence including at least two modifications as compared to SEQ ID NO:12, wherein a first modification is selected from among R228P, R228S, deletion of the sequence RCPEPKSCDTPPPCPRCPEPKSCDTPPPCPRCPEPKSCDTPPPCPR (SEQ ID NO:20) beginning at 228, E233P, L234V, L234F, L235A, deletion of G236, H268Q, Q274K, K276N, Y296F, F300Y, L309V, A327G, A330S, P331S, T339A, R355Q, E356D, M358L, S384N, N392K, M397V, K409R, Q419E, I422V, R435H, F436Y, and P445L. In a further variation, a second modification is selected from among 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N. In various embodiments, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the modifications are in different domains.

In another aspect, the present application is directed to an IgG3 variant including an amino acid sequence having the formula

C-X(227)-X(228)-CPAP-X(233)-X(234)-X(235)-X(236)-X(237)-P-X(239)-X(240)-FLFPP-
X(246)-PKDTLMIS-X(255)-TP-X(258)-V-X(260)-CVV-X(264)-DV-X(267)-X(268)-ED-X(271)-
X(272)-V-X(274)-F-X(276)-W-X(278)-VD-X(281)-V-X(283)-X(284)-HNAKT-X(290)-PR-
X(293)-E-X(295)-X(296)-NST-X(300)-RVV-X(304)-VLTV-X(309)-HQDWLNGKEYKCKV-
X(324)-N-X(326)-X(327)-X(328)-P-X(330)-X(331)-X(332)-X(333)-X(334)-TISK-X(339)-
KGQPREPQVYTLPPS-X(355)-X(356)-E-X(358)-TKNQVSLTCLVKGFYPSDIAVEWES-
X(384)-GQPENNY-X(392)-TTPP-X(397)-LDSDGSFFLYS-X(409)-LTVDKSRWQ-X(419)-GN-
X(422)-FSCSVMHEALHN-X(435)-X(436)-TQKSLSLS-X(445)-GK-,

wherein
-X(227)- is selected from the group consisting of P and G;
-X(228)- is selected from the group consisting of P, R, S, and the sequence

(SEQ ID NO: 20)

RCPEPKSCDTPPPCPRCPEPKSCDTPPPCPRCPEPKSCDTPPPCPR;

-X(233)- is selected from the group consisting of P and E;
-X(234)- is selected from the group consisting of V, LF, Y and I;
-X(235)- is selected from the group consisting of A, L, Y, I and D;
-X(236)- is selected from the group consisting of no amino acid, G, S and A;
-X(237)- is selected from the group consisting of G and D;
-X(239)- is selected from the group consisting of S, D, E, N, Q and T;
-X(240)- is selected from the group consisting of V, I and M;
-X(246)- is selected from the group consisting of K, H and Y;
-X(255)- is selected from the group consisting of R and Y;
-X(258)- is selected from the group consisting of E, H and Y;
-X(260)- is selected from the group consisting of T and H;
-X(264)- is selected from the group consisting of V, I, T and Y;
-X(267)- is selected from the group consisting of S, D and E;
-X(268)- is selected from the group consisting of H, Q, D and E;
-X(271)- is selected from the group consisting of P and G;
-X(272)- is selected from the group consisting of E, Y, H, R and I;
-X(274)- is selected from the group consisting of Q, K and E;
-X(276)- is selected from the group consisting of N and K;
-X(278)- is selected from the group consisting of Y and T;
-X(281)- is selected from the group consisting of G, D and E;
-X(283)- is selected from the group consisting of E, L and H;
-X(284)- is selected from the group consisting of V, E and D;
-X(290)- is selected from the group consisting of K and N;
-X(293)- is selected from the group consisting of E and R;
-X(295)- is selected from the group consisting of Q and E;
-X(296)- is selected from the group consisting of F and Y;
-X(300)- is selected from the group consisting of F and Y;
-X(304)- is selected from the group consisting of S and T;
-X(309)- is selected from the group consisting of V and L;
-X(324)- is selected from the group consisting of S, G and I;
-X(326)- is selected from the group consisting of K and T;
-X(327)- is selected from the group consisting of G, A and D;
-X(328)- is selected from the group consisting of L, A, F, I and T;
-X(330)- is selected from the group consisting of A, S, L, Y and I;
-X(331)- is selected from the group consisting of P and S;
-X(332)- is selected from the group consisting of I, D, E, N, Q and T;
-X(333)- is selected from the group consisting of E and Y;
-X(334)- is selected from the group consisting of K, F, I and T;
-X(339)- is selected from the group consisting of T and A;
-X(355)- is selected from the group consisting of R and Q;
-X(356)- is selected from the group consisting of E and D;
-X(358)- is selected from the group consisting of M and L;
-X(384)- is selected from the group consisting of N and S;
-X(392)- is selected from the group consisting of K and N;
-X(397)- is selected from the group consisting of M and V;
-X(409)- is selected from the group consisting of K and R;
-X(419)- is selected from the group consisting of Q and E;
-X(422)- is selected from the group consisting of V and I;
-X(435)- is selected from the group consisting of H and R;
-X(436)- is selected from the group consisting of Y and F; and
-X(445)- is selected from the group consisting of P and L.

In certain variations, a first modification is selected from among R228P, R228S, deletion of R, deletion of the sequence RCPEPKSCDTPPPCPRCPEPKSCDTPPPCPRCPEPKSCDTPPPCPR (SEQ ID NO:20) beginning at 228, E233P, L234V, L234F, L235A, deletion of G236, H268Q, Q274K, K276N, Y296F, F300Y, L309V, A327G, A330S, P331S, T339A, R355Q, E356D, M358L, S384N, N392K, M397V, K409R, Q419E, 1422V, R435H, F436Y, and P445L. In further variations, a second modification is selected from among 227G, 234Y, 234I, 235Y, 235I, 235D, 236S, 236A, 237D, 239D, 239E, 239N, 239Q, 239T, 240I, 240M, 246H, 246Y, 255Y, 258H, 258Y, 260H, 264I, 264T, 264Y, 267D, 267E, 268D, 268E, 271G, 272Y, 272H, 272R, 272I, 274E, 278T, 281D, 281E, 283L, 283H, 284E, 284D, 290N, 293R, 295E, 304T, 324G, 324I, 326T, 327D, 328A, 328F, 328I, 328T, 330L, 330Y, 330I, 332D, 332E, 332N, 332Q, 332T, 333Y, 334F, 334I, and 334T. In additional embodiments, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the modifications are in different domains. Alternatively, the modifications can be from position 230 until the C terminus.

In another aspect, the present application is directed to an IgG3 variant including an amino acid sequence having the formula:

ASTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTP-X(221)-GD-X(222)-X(223)-
X(224)-X(225)-C-X(227)-X(228)-
CPEPKSCDTPPPCPRCPEPKSCDTPPPCPRCPEPKSCDTPPPCPRC-X(230)-X(231)-
X(232)-X(233)-X(234)-X(235)-X(236)-X(237)-X(238)-X(239)-X(240)-X(241)-L-X(243)-X(244)-
X(245)-X(246)-X(247)-K-X(249)-TLMIS-X(255)-TP-X(258)-V-X(260)-C-X(262)-X(263)-
X(264)-X(265)-X(266)-X(267)-X(268)-X(269)-X(270)-X(271)-X(272)-X(273)-X(274)-X(275)-
X(276)-W-X(278)-V-X(280)-X(281)-X(282)-X(283)-X(284)-X(285)-X(286)-A-X(288)-T-X(290)-
X(291)-X(292)-X(293)-X(294)-X(295)-X(296)-X(297)-X(298)-X(299)-X(300)-X(301)-X(302)-
X(303)-X(304)-X(305)-LTVLHQD-X(313)-LNG-X(317)-X(318)-Y-X(320)-C-X(322)-X(323)-
X(324)-X(325)-X(326)-X(327)-X(328)-X(329)-X(330)-X(331)-X(332)-X(333)-X(334)-X(335)-
X(336)-X(337)-
KTKGQPREPQVYTLPPSREEMTKNQVSLTCLVKGFYPSDIAVEWESSGQPENNYNTTPPM
LDSDGSFFLYSKLTVDKSRWQQGNIFSCSVMHEALHNRFTQKSLSLSPGK,

wherein
-X(221)- is selected from the group consisting of L, K and Y;
-X(222)- is selected from the group consisting of T, E and Y;
-X(223)- is selected from the group consisting of T, E and K;
-X(224)- is selected from the group consisting of H and Y;
-X(225)- is selected from the group consisting of T, E, K and W;
-X(227)- is selected from the group consisting of P, E, G, K and Y;
-X(228)- is selected from the group consisting of R, E, G, K and Y;
-X(230)- is selected from the group consisting of P, A, E, G and Y;
-X(231)- is selected from the group consisting of A, E, G, K, P and Y;
-X(232)- is selected from the group consisting of P, E, G, K and Y;
-X(233)- is selected from the group consisting of E, A, D, F, G, H, I, K, L, M, N, Q, R, S, T, V, W and Y;
-X(234)- is selected from the group consisting of L, D, E, F, G, H, I, K, M, N, P, Q, R, S, T, W and Y;
-X(235)- is selected from the group consisting of L, D, F, G, H, I, K, M, N, P, Q, R, S, T, V, W, and Y;
-X(236)- is selected from the group consisting of G, A, D, E, F, H, I, K, L, M, N, P, Q, R, S, T, V, W and Y;
-X(237)- is selected from the group consisting of G, D, E, F, H, I, K, L, M, N, P, Q, R, S, T, V, W, and Y;
-X(238)- is selected from the group consisting of P, D, E, F, G, H, I, K, L, M, N, Q, R, S, T, V, W and Y;
-X(239)- is selected from the group consisting of S, D, E, F, G, H, I, K, L, M, N, P, Q, R, T, V, W and Y;
-X(240)- is selected from the group consisting of V, A, I, M and T;
-X(241)- is selected from the group consisting of F, D, E, L, R, S, W and Y;
-X(243)- is selected from the group consisting of F, E, H, L, Q, R, W and Y;
-X(244)- is selected from the group consisting of P and H;
-X(245)- is selected from the group consisting of P and A;
-X(246)- is selected from the group consisting of K, D, E, H and Y;
-X(247)- is selected from the group consisting of P, G and V;
-X(249)- is selected from the group consisting of D, H, Q and Y;
-X(255)- is selected from the group consisting of R, E and Y;
-X(258)- is selected from the group consisting of E, H, S and Y;
-X(260)- is selected from the group consisting of T, D, E, H and Y;
-X(262)- is selected from the group consisting of V, A, E, F, I and T;
-X(263)- is selected from the group consisting of V, A, I, M and T;
-X(264)- is selected from the group consisting of V, A, D, E, F, G, H, I, K, L, M, N, P, Q, R, S, T, W and Y;
-X(265)- is selected from the group consisting of D, F, G, H, I, K, L, M, P, Q, R, S, T, V, W and Y;
-X(266)- is selected from the group consisting of V, A, I, M and T;
-X(267)- is selected from the group consisting of S, D, E, F, H, I, K, L, M, N, P, Q, R, V, W and Y;
-X(268)- is selected from the group consisting of H, D, E, F, G, I, K, L, M, P, R, T, V and W;
-X(269)- is selected from the group consisting of E, F, G, H, I, K, L, M, N, P, R, S, T, V, W and Y;
-X(270)- is selected from the group consisting of D, F, G, H, I, L, M, P, Q, R, S, T, W and Y;
-X(271)- is selected from the group consisting of P, A, D, E, F, G, H, I, K, L, M, N, Q, R, S, T, V, W and Y;
-X(272)- is selected from the group consisting of E, D, F, G, H, I, K, L, M, P, R, S, T, V, W and Y;
-X(273)- is selected from the group consisting of V and I;
-X(274)- is selected from the group consisting of Q, D, E, F, G, H, I, L, M, N, P, R, T, V, W and Y;
-X(275)- is selected from the group consisting of F, L and W;
-X(276)- is selected from the group consisting of K, D, E, F, G, H, I, L, M, P, R, S, T, V, W and Y;
-X(278)- is selected from the group consisting of Y, D, E, G, H, I, K, L, M, N, P, Q, R, S, T, V and W;
-X(280)- is selected from the group consisting of D, G, K, L, P and W;
-X(281)- is selected from the group consisting of G, D, E, K, N, P, Q and Y;
-X(282)- is selected from the group consisting of V, E, G, K, P and Y;
-X(283)- is selected from the group consisting of E, G, H, K, L, P, R and Y;
-X(284)- is selected from the group consisting of V, D, E, L, N, Q, T and Y;
-X(285)- is selected from the group consisting of H, D, E, K, Q, W and Y;
-X(286)- is selected from the group consisting of N, E, G, P and Y;
-X(288)- is selected from the group consisting of K, D, E and Y;
-X(290)- is selected from the group consisting of K, D, H, L, N and W;
-X(291)- is selected from the group consisting of P, D, E, G, H, I, Q and T;
-X(292)- is selected from the group consisting of R, D, E, T and Y;
-X(293)- is selected from the group consisting of E, F, G, H, I, L, M, N, P, R, S, T, V, W and Y;
-X(294)- is selected from the group consisting of E, F, G, H, I, K, L, M, P, R, S, T, V, W and Y;
-X(295)- is selected from the group consisting of Q, D, E, F, G, H, I, M, N, P, R, S, T, V, W and Y;
-X(296)- is selected from the group consisting of Y, A, D, E, G, I, K, L, M, N, Q, R, S, T and V;
-X(297)- is selected from the group consisting of N, D, E, F, G, H, I, K, L, M, P, Q, R, S, T, V, W and Y;
-X(298)- is selected from the group consisting of S, E, F, H, I, K, M, Q, R, W and Y;
-X(299)- is selected from the group consisting of T, A, D, E, F, G, H, I, K, L, M, N, P, Q, R, S, V, W and Y;
-X(300)- is selected from the group consisting of F, A, D, E, G, H, K, M, N, P, Q, R, S, T, V and W;
-X(301)- is selected from the group consisting of R, D, E, H and Y;
-X(302)- is selected from the group consisting of V and I;
-X(303)- is selected from the group consisting of V, D, E and Y;
-X(304)- is selected from the group consisting of S, D, H, L, N and T;
-X(305)- is selected from the group consisting of V, E, T and Y;
-X(313)- is selected from the group consisting of W and F;
-X(317)- is selected from the group consisting of K, E and Q;
-X(318)- is selected from the group consisting of E, H, L, Q, R and Y;
-X(320)- is selected from the group consisting of K, D, F, G, H, I, L, N, P, S, T, V, W and Y;
-X(322)- is selected from the group consisting of K, D, F, G, H, I, P, S, T, V, W and Y;
-X(323)- is selected from the group consisting of V and I;
-X(324)- is selected from the group consisting of S, D, F, G, H, I, L, M, P, R, T, V, W and Y;
-X(325)- is selected from the group consisting of N, A, D, E, F, G, H, I, K, L, M, P, Q, R, S, T, V, W and Y;
-X(326)- is selected from the group consisting of K, I, L, P and T;
-X(327)- is selected from the group consisting of A, D, E, F, H, I, K, L, M, N, P, R, T, V, W and Y;
-X(328)- is selected from the group consisting of L, A, D, E, F, G, H, I, K, M, N, P, Q, R, S, T, V, W and Y;
-X(329)- is selected from the group consisting of P, D, E, F, G, H, I, K, L, M, N, Q, R, S, T, V, W and Y;
-X(330)- is selected from the group consisting of A, E, F, G, H, I, L, M, N, P, R, T, V, W and Y;
-X(331)- is selected from the group consisting of P, D, F, H, I, L, M, Q, R, T, V, W and Y;
-X(332)- is selected from the group consisting of I, A, D, E, F, H, K, L, M, N, P, Q, R, S, T, V, W and Y;
-X(333)- is selected from the group consisting of E, F, H, I, L, M, P, T and Y;
-X(334)- is selected from the group consisting of K, F, I, P and T;
-X(335)- is selected from the group consisting of T, D, F, G, H, I, L, M, N, P, R, S, V, W and Y;
-X(336)- is selected from the group consisting of I, E, K and Y; and
-X(337)- is selected from the group consisting of S, E, H and N.

In certain variations, the variant differs from SEQ ID NO:12 by at least one amino acid.

In another aspect, the present application is directed to an IgG3 variant including an amino acid sequence having the formula:

ASTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTP-X(221)-GDTTHTC-X(227)-
RCPEPKSCDTPPPCPRCPEPKSCDTPPPCPRCPEPKSCDTPPPCPRCPAPE-X(234)-
X(235)-X(236)-X(237)-P-X(239)-X(240)-FLFPP-X(246)-PKDTLMIS-X(255)-TP-X(258)-V-
X(260)-CVV-X(264)-DV-X(267)-X(268)-ED-X(271)-X(272)-V-X(274)-FKW-X(278)-VD-
X(281)-V-X(283)-X(284)-HNAKT-X(290)-PR-X(293)-E-X(295)-YNSTFRVV-X(304)-
VLTVLHQDWLNGKEYKCKV-X(324)-N-X(326)-X(327)-X(328)-P-X(330)-P-X(332)-X(333)-
X(334)-
TISKTKGQPREPQVYTLPPSREEMTKNQVSLTCLVKGFYPSDIAVEWESSGQPENNYNTTP
PMLDSDGSFFLYSKLTVDKSRWQQGNIFSCSVMHEALHNRFTQKSLSLSPGK-;

wherein
-X(221)- is selected from the group consisting of L and K;
-X(227)- is selected from the group consisting of P and G;
-X(234)- is selected from the group consisting of L, Y and I;
-X(235)- is selected from the group consisting of L, Y, I and D;
-X(236)- is selected from the group consisting of G, S and A;
-X(237)- is selected from the group consisting of G and D;
-X(239)- is selected from the group consisting of S, D, E, N, Q and T;
-X(240)- is selected from the group consisting of V, I and M;
-X(246)- is selected from the group consisting of K, H and Y;
-X(255)- is selected from the group consisting of R and Y;
-X(258)- is selected from the group consisting of E, H and Y;
-X(260)- is selected from the group consisting of T and H;
-X(264)- is selected from the group consisting of V, I, T and Y;
-X(267)- is selected from the group consisting of S, D and E;
-X(268)- is selected from the group consisting of H, D and E;
-X(271)- is selected from the group consisting of P and G;
-X(272)- is selected from the group consisting of E, Y, H, R and I;
-X(274)- is selected from the group consisting of Q and E;
-X(278)- is selected from the group consisting of Y and T;
-X(281)- is selected from the group consisting of G, D and E;
-X(283)- is selected from the group consisting of E, L and H;
-X(284)- is selected from the group consisting of V, E and D;
-X(290)- is selected from the group consisting of K and N;
-X(293)- is selected from the group consisting of E and R;
-X(295)- is selected from the group consisting of Q and E;
-X(304)- is selected from the group consisting of S and T;
-X(324)- is selected from the group consisting of S, G and I;
-X(326)- is selected from the group consisting of K and T;
-X(327)- is selected from the group consisting of A and D;
-X(328)- is selected from the group consisting of L, A, F, I and T;
-X(330)- is selected from the group consisting of A, L, Y and I;
-X(332)- is selected from the group consisting of I, D, E, N, Q and T;
-X(333)- is selected from the group consisting of E and Y; and
-X(334)- is selected from the group consisting of K, F, I and T.

In certain variations, the variant differs from SEQ ID NO:12 by at least one amino acid. In additional variations, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the modifications are in different domains.

In another aspect, the present application is directed to an IgG4 variant including two or more amino acid modifications as compared to SEQ ID NO:13. The modifications can be selected from among C131S, R133K, E137G, S138G, S192N, L193F, K196Q, T199I, D203N, R214K, R214T, S217P, S217R, S217L, Y219S, Y219C, Y219T, G220C, G220P, -221D, -221L, insertion of the sequence LGD at -221, -222K, -222V, -222T, -223T, P224H, P224E, P225T, P225-, S228P, S228R, substitution of the sequence RCPEPKSCDTPPPCPRCPEPKSCDTPPPCPRCPEPKSCDTPPPCPR at 228, E233P, F234L, F234V, L235A, G236-, Q268H, Q274K, N276K, F296Y, Y300F, L309V, G327A, S330A, S331P, A339T, Q355R, E356D, M358L, N384S, K392N, V397M, R409K, E419Q, V422I, H435R, Y436F, and L445P. In certain embodiments, at least two of the amino acid modifications are in different domains. In various embodiments, the formula has at least 1, 2, 3, 4, 5, 6, 7, 8, 9, 10 or more amino acid modifications as compared to an amino acid sequence including SEQ ID NO:14. In additional embodiments, at least 2, 3, or 4 of the modifications are in different domains.

In another aspect, the present application is directed to an IgG4 variant including an amino acid sequence having the formula:

ASTKGPSVFPLAP-X(131)-S-X(133)-STS-X(137)-X(138)-
TAALGCLVKDYFPEPVTVSWNSGALTSGVHTFPAVLQSSGLYSLSSVVTVPSSSLGT-
X(196)-TY-X(199)-CNV-X(203)-HKPSNTKVDK-X(214)-VE-X(217)-K-X(219)-X(220)-X(221)-
X(222)-X(223)-X(224)-X(225)-CP-X(228)-CPAPE-X(234)-
LGGPSVFLFPPKPKDTLMISRTPEVTCVVVDVS-X(268)-EDPEV-X(274)-
FNWYVDGVEVHNAKTKPREEQ-X(296)-NSTYRVVSVLTVLHQDWLNGKEYKCKVSNK-
X(327)-LP-X(330)-X(331)-IEKTISKAKGQPREPQVYTLPPS-X(355)-X(356)-E-X(358)-
TKNQVSLTCLVKGFYPSDIAVEWESNGQPENNYKTTPPVLDSDGSFFLYS-X(409)-
LTVDKSRWQ-X(419)-GNVFSCSVMHEALHNHYTQKSLSLS-X(445)-GK

wherein
X(131) is selected from the group consisting of C and S;
X(133) is selected from the group consisting of R and K;
X(137) is selected from the group consisting of E and G;
X(138) is selected from the group consisting of S and G;
X(196) is selected from the group consisting of K and Q;
X(199) is selected from the group consisting of T and I;
X(203) is selected from the group consisting of D and N;
X(214) is selected from the group consisting of R and K;
X(217) is selected from the group consisting of S and P;
X(219) is selected from the group consisting of Y and S;
X(220) is selected from the group consisting of G and C;
X(221) is selected from the group consisting of no amino acid and D;
X(222) is selected from the group consisting of no amino acid and K;
X(223) is selected from the group consisting of no amino acid and T;
X(224) is selected from the group consisting of P and H;
X(225) is selected from the group consisting of P and T;
X(228) is selected from the group consisting of S and P;
X(234) is selected from the group consisting of F and L;
X(268) is selected from the group consisting of Q and H;
X(274) is selected from the group consisting of Q and K;
X(296) is selected from the group consisting of F and Y;
X(327) is selected from the group consisting of G and A;
X(330) is selected from the group consisting of S and A;
X(331) is selected from the group consisting of S and P;
X(355) is selected from the group consisting of Q and R;
X(356) is selected from the group consisting of E and D;
X(358) is selected from the group consisting of M and L;
X(409) is selected from the group consisting of R and K;
X(419) is selected from the group consisting of E and Q; and
X(445) is selected from the group consisting of L and P.

In certain embodiments, at least two of the amino acid modifications are in different domains. In various embodiments, the formula has at least 1, 2, 3, 4, 5, 6, 7, 8, 9, 10 or more amino acid modifications as compared to an amino acid sequence including SEQ ID NO:13. In additional embodiments, at least 2, 3, or 4 of the modifications are in different domains.

In another aspect, the present application is directed to an IgG4 variant including two or more amino acid modifications as compared to SEQ ID NO:14. In certain embodiments, the modifications selected from among C131S, R133K, E137G, S138G, K196Q, T1991, D203N, R214K, S217P, Y219S, G220C, 221D, -222K, -223T, P224H, P225T, S228P, F234L, Q268H, Q274K, F296Y, G327A, S330A, S331P, Q355R, E356D, M358L, R409K, E419Q, and L445P. In various embodiments, the formula has at least 1, 2, 3, 4, 5, 6, 7, 8, 9, 10 or more amino acid modifications as compared to an amino acid sequence including SEQ ID NO:13. In additional embodiments, at least 2, 3, or 4 of the modifications are in different domains.

In another aspect, the present application is directed to an IgG4 variant including an amino acid sequence having the formula

-ASTKGPSVFPLAP-X(131)-S-X(133)-STS-X(137)-X(138)-
TAALGCLVKDYFPEPVTVSWNSGALTSGVHTFPAVLQSSGLYSLSSVVTVPSS-X(192)-
X(193)-GT-X(196)-TY-X(199)-CNV-X(203)-HKPSNTKVDK-X(214)-VE-X(217)-K-X(219)-
X(220)-X(221)-X(222)-X(223)-X(224)-X(225)-C-X(227)-X(228)-C-X(230)-X(231)-X(232)-
X(233)-X(234)-X(235)-X(236)-X(237)-X(238)-X(239)-X(240)-X(241)-L-X(243)-X(244)-X(245)-
X(246)-X(247)-K-X(249)-TLMIS-X(255)-TP-X(258)-V-X(260)-C-X(262)-X(263)-X(264)-
X(265)-X(266)-X(267)-X(268)-X(269)-X(270)-X(271)-X(272)-X(273)-X(274)-X(275)-X(276)-
W-X(278)-V-X(280)-X(281)-X(282)-X(283)-X(284)-X(285)-X(286)-A-X(288)-T-X(290)-X(291)-
X(292)-X(293)-X(294)-X(295)-X(296)-X(297)-X(298)-X(299)-X(300)-X(301)-X(302)-X(303)-
X(304)-X(305)-LTV-X(309)-HQD-X(313)-LNG-X(317)-X(318)-Y-X(320)-C-X(322)-X(323)-
X(324)-X(325)-X(326)-X(327)-X(328)-X(329)-X(330)-X(331)-X(332)-X(333)-X(334)-X(335)-
X(336)-X(337)-K-X(339)-KGQPREPQVYTLPPS-X(355)-X(356)-E-X(358)-
TKNQVSLTCLVKGFYPSDIAVEWES-X(384)-GQPENNY-X(392)-TTPP-X(397)-
LDSDGSFFLYS-X(409)-LTVDKSRWQ-X(419)-GN-X(422)-FSCSVMHEALHN-X(435)-
X(436)-TQKSLSLS-X(445)-GK

wherein
-X(131)- is selected from the group consisting of C and S;
-X(133)- is selected from the group consisting of R and K;
-X(137)- is selected from the group consisting of E and G;
-X(138)- is selected from the group consisting of S and G;
-X(192)- is selected from the group consisting of N and S;
-X(193)- is selected from the group consisting of F and L;
-X(196)- is selected from the group consisting of Q and K;
-X(199)- is selected from the group consisting of T and I;
-X(203)- is selected from the group consisting of D and N;
-X(214)- is selected from the group consisting of T, K and R;
-X(217)- is selected from the group consisting of R, P, L and S;
-X(219)- is selected from the group consisting of C, S, T and Y;
-X(220)- is selected from the group consisting of C, P and G;
-X(221)- is selected from the group consisting of no amino acid, D, K, Y, L, and the sequence LGD;
-X(222)- is selected from the group consisting of V, K, T, no amino acid, E and Y;
-X(223)- is selected from the group consisting of no amino acid, T, E and K;
-X(224)- is selected from the group consisting of E, H, P and Y;
-X(225)- is selected from the group consisting of no amino acid, T, P, E, K and W;
-X(227)- is selected from the group consisting of P, E, G, K and Y;
-X(228)- is selected from the group consisting of P, S, E, G, K, Y, R, and the sequence

(SEQ ID NO: 20)

RCPEPKSCDTPPPCPRCPEPKSCDTPPPCPRCPEPKSCDTPPPCPR;

-X(230)- is selected from the group consisting of P, A, E, G and Y;
-X(231)- is selected from the group consisting of A, E, G, K, P and Y;
-X(232)- is selected from the group consisting of P, E, G, K and Y;
-X(233)- is selected from the group consisting of P, E, A, D, F, G, H, I, K, L, M, N, Q, R, S, T, V, W and Y;
-X(234)- is selected from the group consisting of V, L, F, D, E, F, G, H, I, K, M, N, P, Q, R, S, T, W and Y;
-X(235)- is selected from the group consisting of A, L, D, F, G, H, I, K, M, N, P, Q, R, S, T, V, W, and Y;
-X(236)- is selected from the group consisting of no amino acid, G, A, D, E, F, H, I, K, L, M, N, P, Q, R, S, T, V, W and Y;
-X(237)- is selected from the group consisting of G, D, E, F, H, I, K, L, M, N, P, Q, R, S, T, V, W and Y;
-X(238)- is selected from the group consisting of P, D, E, F, G, H, I, K, L, M, N, Q, R, S, T, V, W and Y;
-X(239)- is selected from the group consisting of S, D, E, F, G, H, I, K, L, M, N, P, Q, R, T, V, W and Y;
-X(240)- is selected from the group consisting of V, A, I, M and T;
-X(241)- is selected from the group consisting of F, D, E, L, R, S, W and Y;
-X(243)- is selected from the group consisting of F, E, H, L, Q, R, and Y;
-X(244)- is selected from the group consisting of P and H;
-X(245)- is selected from the group consisting of P and A;
-X(246)- is selected from the group consisting of, K, D, E, H and Y;
-X(247)- is selected from the group consisting of P, G and V;
-X(249)- is selected from the group consisting of D, H, Q and Y;
-X(255)- is selected from the group consisting of R and Y;
-X(258)- is selected from the group consisting of E, H, S and Y;
-X(260)- is selected from the group consisting of T, D, E, H and Y;
-X(262)- is selected from the group consisting of V, A, E, F, I and T;
-X(263)- is selected from the group consisting of V, A, I, M and T;
-X(264)- is selected from the group consisting of V, A, D, E, F, G, H, I, K, L, M, N, P, Q, R, S, T, W, and Y;
-X(265)- is selected from the group consisting of D, F, G, H, I, K, L, M, P, Q, R, S, T, V, W and Y;
-X(266)- is selected from the group consisting of V, A, I, M and T;
-X(267)- is selected from the group consisting of S, D, E, F, H, I, K, L, M, N, P, Q, R, V, W and Y;
-X(268)- is selected from the group consisting of H, Q, D, E, F, G, I, K, L, M, P, R, T, V and W;
-X(269)- is selected from the group consisting of E, F, G, H, I, K, L, M, N, P, R, S, T, V, W and Y;
-X(270)- is selected from the group consisting of D, F, G, H, I, L, M, P, Q, R, S, T, W and Y;
-X(271)- is selected from the group consisting of P, A, D, E, F, G, H, I, K, L, M, N, Q, R, S, T, V, W and Y;
-X(272)- is selected from the group consisting of E, D, F, G, H, I, K, L, M, P, R, S, T, V, W and Y;
-X(273)- is selected from the group consisting of V and I;
-X(274)- is selected from the group consisting of Q, K, D, E, F, G, H, I, L, M, N, P, R, T, V, W and Y;
-X(275)- is selected from the group consisting of FL and W;
-X(276)- is selected from the group consisting of N, K, D, E, F, G, H, I, L, M, P, R, S, T, V, W and Y;
-X(278)- is selected from the group consisting of Y, D, E, G, H, I, K, L, M, N, P, Q, R, S, T, V and W;
-X(280)- is selected from the group consisting of D, G, K, L, P and W;
-X(281)- is selected from the group consisting of G, D, E, K, N, P, Q and Y;
-X(282)- is selected from the group consisting of V, E, G, K, P and Y;
-X(283)- is selected from the group consisting of E, G, H, K, L, P, R and Y;
-X(284)- is selected from the group consisting of V, D, E, L, N, Q, T and Y;
-X(285)- is selected from the group consisting of H, D, E, K, Q, W and Y;
-X(286)- is selected from the group consisting of N, E, G, P and Y;
-X(288)- is selected from the group consisting of K, D, E and Y;
-X(290)- is selected from the group consisting of K, D, H, L, N and W;
-X(291)- is selected from the group consisting of P, D, E, G, H, I, Q and T;
-X(292)- is selected from the group consisting of R, D, E, T and Y;
-X(293)- is selected from the group consisting of E, F, G, H, I, L, M, N, P, R, S, T, V, W and Y;
-X(294)- is selected from the group consisting of E, F, G, H, I, K, L, M, P, R, S, T, V, W and Y;
-X(295)- is selected from the group consisting of Q, D, E, F, G, H, I, M, N, P, R, S, T, V, W and Y;
-X(296)- is selected from the group consisting of F, Y, A, D, E, G, I, K, L, M, N, Q, R, S, T and V;
-X(297)- is selected from the group consisting of N, D, E, F, G, H, I, K, L, M, P, Q, R, S, T, V, W and Y;
-X(298)- is selected from the group consisting of S, E, F, H, I, K, M, Q, R, W and Y;
-X(299)- is selected from the group consisting of T, A, D, E, F, G, H, I, K, L, M, N, P, Q, R, S, V, W and Y;
-X(300)- is selected from the group consisting of F, Y, A, D, E, G, H, K, M, N, P, Q, R, S, T, V and W;
-X(301)- is selected from the group consisting of R, D, E, H and Y;
-X(302)- is selected from the group consisting of V and I;
-X(303)- is selected from the group consisting of V, D, E and Y;
-X(304)- is selected from the group consisting of S, D, H, L, N and T;
-X(305)- is selected from the group consisting of V, E, T and Y;
-X(309)- is selected from the group consisting of V and L;
-X(313)- is selected from the group consisting of W and F;
-X(317)- is selected from the group consisting of K, E and Q;
-X(318)- is selected from the group consisting of E, H, L, Q, R and Y;
-X(320)- is selected from the group consisting of K, D, F, G, H, I, L, N, P, S, T, V, W and Y;
-X(322)- is selected from the group consisting of K, D, F, G, H, I, P, S, T, V, W and Y;
-X(323)- is selected from the group consisting of V and I;
-X(324)- is selected from the group consisting of S, D, F, G, H, I, L, M, P, R, T, V, W and Y;
-X(325)- is selected from the group consisting of N, A, D, E, F, G, H, I, K, L, M, P, Q, R, S, T, V, W and Y;
-X(326)- is selected from the group consisting of K, I, L, P and T;
-X(327)- is selected from the group consisting of G, A, D, E, F, H, I, K, L, M, N, P, R, T, V, W and Y;
-X(328)- is selected from the group consisting of L, A, D, E, F, G, H, I, K, M, N, P, Q, R, S, T, V, W and Y;
-X(329)- is selected from the group consisting of P, D, E, F, G, H, I, K, L, M, N, Q, R, S, T, V, W and Y;
-X(330)- is selected from the group consisting of A, S, E, F, G, H, I, L, M, N, P, R, T, V, W and Y;
-X(331)- is selected from the group consisting of P, S, D, F, H, I, L, M, Q, R, T, V, W and Y;
-X(332)- is selected from the group consisting of I, A, D, E, F, H, K, L, M, N, P, Q, R, S, T, V, W and Y;
-X(333)- is selected from the group consisting of E, F, H, I, L, M, P, T and Y;
-X(334)- is selected from the group consisting of K, F, I, P and T;
-X(335)- is selected from the group consisting of T, D, F, G, H, I, L, M, N, P, R, S, V, W and Y;
-X(336)- is selected from the group consisting of I, E, K and Y;
-X(337)- is selected from the group consisting of S, E, H and N;
-X(339)- is selected from the group consisting of T and A;
-X(355)- is selected from the group consisting of R and Q;
-X(356)- is selected from the group consisting of E and D;
-X(358)- is selected from the group consisting of M and L;
-X(384)- is selected from the group consisting of N and S;
-X(392)- is selected from the group consisting of K and N;
-X(397)- is selected from the group consisting of M and V;
-X(409)- is selected from the group consisting of K and R;
-X(419)- is selected from the group consisting of Q and E;
-X(422)- is selected from the group consisting of V and I;
-X(435)- is selected from the group consisting of H and R;
-X(436)- is selected from the group consisting of Y and F; and
-X(445)- is selected from the group consisting of P and L.

In one variation, a first modification is selected from among C131S, R133K, E137G, S138G, S192N, L193F, K196Q, T199I, D203N, R214K, R214T, S217P, S217R, S217L, Y219S, Y219C, Y219T, G220C, G220P, -221D, -221L, insertion of the sequence LGD at -221, -222K, -222V, -222T, -223T, P224H, P224E, P225T, P225-, S228P, S228R, substitution of the sequence RCPEPKSCDTPPPCPRCPEPKSCDTPPPCPRCPEPKSCDTPPPCPR (SEQ ID NO:20) at 228, E233P, F234L, F234V, L235A, G236-, Q268H, Q274K, N276K, F296Y, Y300F, L309V, G327A, S330A, S331P, A339T, Q355R, E356D, M358L, N384S, K392N, V397M, R409K, E419Q, V422I, H435R, Y436F, and L445P. In a further variation, a second modification is selected from among 221K, 221Y, 222E, 222Y, 223E, 223K, 224Y, 225E, 225K, 225W, 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N.

In a further aspect, the present application is directed to an IgG4 variant amino acid sequence having at least two amino acid modifications as compared to SEQ ID NO:13. The IgG4 variant includes a first modification selected from among C131S, R133K, E137G, S138G, S192N, L193F, K196Q, T199I, D203N, R214K, R214T, S217P, S217R, S217L, Y219S, Y219C, Y219T, G220C, G220P, -221 D, -221L, insertion of the sequence LGD at -221, -222K, -222V, -222T, -223T, P224H, P224E, P225T, P225-, S228P, S228R, substitution of the sequence RCPEPKSCDTPPPCPRCPEPKSCDTPPPCPRCPEPKSCDTPPPCPR (SEQ ID NO:20) at 228, E233P, F234L, F234V, L235A, G236-, Q268H, Q274K, N276K, F296Y, Y300F, L309V, G327A, S330A, S533P, A339T, Q355R, E356D, M358L, N384S, K392N, V397M, R409K, E419Q, V422I, H435R, Y436F, and L445P. In a further variation, a second modification is selected from among 221K, 221Y, 222E, 222Y, 223E, 223K, 224Y, 225E, 225K, 225W, 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N.

In another aspect, the present application is directed to an IgG4 variant including an amino acid sequence having the formula:

ASTKGPSVFPLAP-X(131)-S-X(133)-STS-X(137)-X(138)-
TAALGCLVKDYFPEPVTVSWNSGALTSGVHTFPAVLQSSGLYSLSSVVTVPSS-X(192)-
X(193)-GT-X(196)-TY-X(199)-CNV-X(203)-HKPSNTKVDK-X(214)-VE-X(217)-K-X(219)-
X(220)-X(221)-X(222)-X(223)-X(224)-X(225)-C-X(227)-X(228)-CPAP-X(233)-X(234)-X(235)-
X(236)-X(237)-P-X(239)-X(240)-FLFPP-X(246)-PKDTLMIS-X(255)-TP-X(258)-V-X(260)-
CVV-X(264)-DV-X(267)-X(268)-ED-X(271)-X(272)-V-X(274)-F-X(276)-W-X(278)-VD-X(281)-
V-X(283)-X(284)-HNAKT-X(290)-PR-X(293)-E-X(295)-X(296)-NST-X(300)-RVV-X(304)-
VLTV-X(309)-HQDWLNGKEYKCKV-X(324)-N-X(326)-X(327)-X(328)-P-X(330)-X(331)-
X(332)-X(333)-X(334)-TISK-X(339)-KGQPREPQVYTLPPS-X(355)-X(356)-E-X(358)-
TKNQVSLTCLVKGFYPSDIAVEWES-X(384)-GQPENNY-X(392)-TTPP-X(397)-
LDSDGSFFLYS-X(409)-LTVDKSRWQ-X(419)-GN-X(422)-FSCSVMHEALHN-X(435)-
X(436)-TQKSLSLS-X(445)-GK;

wherein
-X(131)- is selected from the group consisting of C and S;
-X(133)- is selected from the group consisting of R and K;
-X(137)- is selected from the group consisting of E and G;
-X(138)- is selected from the group consisting of S and G;
-X(192)- is selected from the group consisting of N and S;
-X(193)- is selected from the group consisting of F and L;
-X(196)- is selected from the group consisting of Q and K;
-X(199)- is selected from the group consisting of T and I;
-X(203)- is selected from the group consisting of D and N;
-X(214)- is selected from the group consisting of T, K and R;
-X(217)- is selected from the group consisting of R, P, L and S;
-X(219)- is selected from the group consisting of C, S, T and Y;
-X(220)- is selected from the group consisting of C, P and G;
-X(221)- is selected from the group consisting of no amino acid, D, L, K, and the sequence LGD;
-X(222)- is selected from the group consisting of V, K, T, and no amino acid;
-X(223)- is selected from the group consisting of no amino acid and T;
-X(224)- is selected from the group consisting of E, H and P;
-X(225)- is selected from the group consisting of no amino acid, T and P;
-X(227)- is selected from the group consisting of P and G;
-X(228)- is selected from the group consisting of P, R, S, and the sequence

(SEQ ID NO: 20)

RCPEPKSCDTPPPCPRCPEPKSCDTPPPCPRCPEPKSCDTPPPCPR;

-X(233)- is selected from the group consisting of P and E;
-X(234)- is selected from the group consisting of V, LF, Y and I;
-X(235)- is selected from the group consisting of A, L, Y, I and D;
-X(236)- is selected from the group consisting of no amino acid, G, S and A;
-X(237)- is selected from the group consisting of G and D;
-X(239)- is selected from the group consisting of S, D, E, N, Q and T;
-X(240)- is selected from the group consisting of V, I and M;
-X(246)- is selected from the group consisting of K, H and Y;
-X(255)- is selected from the group consisting of R and Y;
-X(258)- is selected from the group consisting of E, H and Y;
-X(260)- is selected from the group consisting of T and H;
-X(264)- is selected from the group consisting of V, I, T and Y;
-X(267)- is selected from the group consisting of S, D and E;
-X(268)- is selected from the group consisting of H, Q, D and E;
-X(271)- is selected from the group consisting of P and G;
-X(272)- is selected from the group consisting of E, Y, H, R and I;
-X(274)- is selected from the group consisting of Q, K and E;
-X(276)- is selected from the group consisting of N and K;
-X(278)- is selected from the group consisting of Y and T;
-X(281)- is selected from the group consisting of G, D and E;
-X(283)- is selected from the group consisting of E, L and H;
-X(284)- is selected from the group consisting of V, E and D;
-X(290)- is selected from the group consisting of K and N;
-X(293)- is selected from the group consisting of E and R;
-X(295)- is selected from the group consisting of Q and E;
-X(296)- is selected from the group consisting of F and Y;
-X(300)- is selected from the group consisting of F and Y;
-X(304)- is selected from the group consisting of S and T;
-X(309)- is selected from the group consisting of V and L;
-X(324)- is selected from the group consisting of S, G and I;
-X(326)- is selected from the group consisting of K and T;
-X(327)- is selected from the group consisting of G, A and D;
-X(328)- is selected from the group consisting of L, A, F, I and T;
-X(330)- is selected from the group consisting of A, S, L, Y and I;
-X(331)- is selected from the group consisting of P and S;
-X(332)- is selected from the group consisting of I, D, E, N, Q and T;
-X(333)- is selected from the group consisting of E and Y;
-X(334)- is selected from the group consisting of K, F, I and T;
-X(339)- is selected from the group consisting of T and A;
-X(355)- is selected from the group consisting of R and Q;
-X(356)- is selected from the group consisting of E and D;
-X(358)- is selected from the group consisting of M and L;
-X(384)- is selected from the group consisting of N and S;
-X(392)- is selected from the group consisting of K and N;
-X(397)- is selected from the group consisting of M and V;
-X(409)- is selected from the group consisting of K and R;
-X(419)- is selected from the group consisting of Q and E;
-X(422)- is selected from the group consisting of V and I;
-X(435)- is selected from the group consisting of H and R;
-X(436)- is selected from the group consisting of Y and F; and
-X(445)- is selected from the group consisting of P and L.

In one variation, a first modification is selected from among C131S, R133K, E137G, S138G, S192N, L193F, K196Q, T199I, D203N, R214K, R214T, S217P, S217R, S217L, Y219S, Y219C, Y219T, G220C, G220P, -221D, -221L, insertion of the sequence LGD at -221, -222K, -222V, -222T, -223T, P224H, P224E, P225T, P225-, S228P, S228R, substitution of the sequence RCPEPKSCDTPPPCPRCPEPKSCDTPPPCPRCPEPKSCDTPPPCPR (SEQ ID NO:20) at 228, E233P, F234L, F234V, L235A, G236-, Q268H, Q274K, N276K, F296Y, Y300F, L309V, G327A, S330A, S331P, A339T, Q355R, E356D, M358L, N384S, K392N, V397M, R409K, E419Q, V4221, H435R, Y436F, and L445P. In a further variation, a second modification is selected from among 227G, 234Y, 234I, 235Y, 235I, 235D, 236S, 236A, 237D, 239D, 239E, 239N, 239Q, 239T, 240I, 240M, 246H, 246Y, 255Y, 258H, 258Y, 260H, 264I, 264T, 264Y, 267D, 267E, 268D, 268E, 271G, 272Y, 272H, 272R, 272I, 274E, 278T, 281D, 281E, 283L, 283H, 284E, 284D, 290N, 293R, 295E, 304T, 324G, 324I, 326T, 327D, 328A, 328F, 328I, 328T, 330L, 330Y, 330I, 332D, 332E, 332N, 332Q, 332T, 333Y, 334F, 3341, and 334T.

In another aspect, the present application is directed to an IgG4 variant including an amino acid sequence having the formula:

-C-X(227)-X(228)-C-X(230)-X(231)-X(232)-X(233)-X(234)-X(235)-X(236)--X(237)-X(238)-
X(239)-X(240)-X(241)-L-X(243)-X(244)-X(245)-X(246)-X(247)-K-X(249)-TLMIS-X(255)-TP-
X(258)-V-X(260)-C-X(262)-X(263)-X(264)-X(265)-X(266)-X(267)-X(268)-X(269)-X(270)-
X(271)-X(272)-X(273)-X(274)-X(275)-X(276)-W-X(278)-V-X(280)-X(281)-X(282)-X(283)-
X(284)-X(285)-X(286)-A-X(288)-T-X(290)-X(291)-X(292)-X(293)-X(294)-X(295)-X(296)-
X(297)-X(298)-X(299)-X(300)-X(301)-X(302)-X(303)-X(304)-X(305)-LTV-X(309)-HQD-
X(313)-LNG-X(317)-X(318)-Y-X(320)-C-X(322)-X(323)-X(324)-X(325)-X(326)-X(327)-
X(328)-X(329)-X(330)-X(331)-X(332)-X(333)-X(334)-X(335)-X(336)-X(337)-K-X(339)-
KGQPREPQVYTLPPS-X(355)-X(356)-E-X(358)-TKNQVSLTCLVKGFYPSDIAVEWES-
X(384)-GQPENNY-X(392)-TTPP-X(397)-LDSDGSFFLYS-X(409)-LTVDKSRWQ-X(419)-GN-
X(422)-FSCSVMHEALHN-X(435)-X(436)-TQKSLSLS-X(445)-GK

wherein
-X(227)- is selected from the group consisting of P, E, G, K and Y;
-X(228)- is selected from the group consisting of P, S, E, G, K, Y, R, and the sequence

(SEQ ID NO: 20)

RCPEPKSCDTPPPCPRCPEPKSCDTPPPCPRCPEPKSCDTPPPCPR;

-X(230)- is selected from the group consisting of P, A, E, G and Y;
-X(231)- is selected from the group consisting of A, E, G, K, P and Y;
-X(232)- is selected from the group consisting of P, E, G, K and Y;
-X(233)- is selected from the group consisting of P, E, A, D, F, G, H, I, K, L, M, N, Q, R, S, T, V, W and Y;
-X(234)- is selected from the group consisting of V, L, F, D, E, F, G, H, I, K, M, N, P, Q, R, S, T, W and Y;
-X(235)- is selected from the group consisting of A, L, D, F, G, H, I, K, M, N, P, Q, R, S, T, V, W, and Y;
-X(236)- is selected from the group consisting of no amino acid, G, A, D, E, F, H, I, K, L, M, N, P, Q, R, S, T, V, W and Y;
-X(237)- is selected from the group consisting of G, D, E, F, H, I, K, L, M, N, P, Q, R, S, T, V, W and Y;
-X(238)- is selected from the group consisting of P, D, E, F, G, H, I, K, L, M, N, Q, R, S, T, V, W and Y;
-X(239)- is selected from the group consisting of S, D, E, F, G, H, I, K, L, M, N, P, Q, R, T, V, W and Y;
-X(240)- is selected from the group consisting of V, A, I, M and T;
-X(241)- is selected from the group consisting of F, D, E, L, R, S, W and Y;
-X(243)- is selected from the group consisting of F, E, H, L, Q, R, and Y;
-X(244)- is selected from the group consisting of P and H;
-X(245)- is selected from the group consisting of P and A;
-X(246)- is selected from the group consisting of, K, D, E, H and Y;
-X(247)- is selected from the group consisting of P, G and V;
-X(249)- is selected from the group consisting of D, H, Q and Y;
-X(255)- is selected from the group consisting of R and Y;
-X(258)- is selected from the group consisting of E, H, S and Y;
-X(260)- is selected from the group consisting of T, D, E, H and Y;
-X(262)- is selected from the group consisting of V, A, E, F, I and T;
-X(263)- is selected from the group consisting of V, A, I, M and T;
-X(264)- is selected from the group consisting of V, A, D, E, F, G, H, I, K, L, M, N, P, Q, R, S, T, W, and Y;
-X(265)- is selected from the group consisting of D, F, G, H, I, K, L, M, P, Q, R, S, T, V, W and Y;
-X(266)- is selected from the group consisting of V, A, I, M and T;
-X(267)- is selected from the group consisting of S, D, E, F, H, I, K, L, M, N, P, Q, R, V, W and Y;
-X(268)- is selected from the group consisting of H, Q, D, E, F, G, I, K, L, M, P, R, T, V and W;
-X(269)- is selected from the group consisting of E, F, G, H, I, K, L, M, N, P, R, S, T, V, W and Y;
-X(270)- is selected from the group consisting of D, F, G, H, I, L, M, P, Q, R, S, T, W and Y;
-X(271)- is selected from the group consisting of P, A, D, E, F, G, H, I, K, L, M, N, Q, R, S, T, V, W and Y;
-X(272)- is selected from the group consisting of E, D, F, G, H, I, K, L, M, P, R, S, T, V, W and Y;
-X(273)- is selected from the group consisting of V and I;
-X(274)- is selected from the group consisting of Q, K, D, E, F, G, H, I, L, M, N, P, R, T, V, W and Y;
-X(275)- is selected from the group consisting of FL and W;
-X(276)- is selected from the group consisting of N, K, D, E, F, G, H, I, L, M, P, R, S, T, V, W and Y;
-X(278)- is selected from the group consisting of Y, D, E, G, H, I, K, L, M, N, P, Q, R, S, T, V and W;
-X(280)- is selected from the group consisting of D, G, K, L, P and W;
-X(281)- is selected from the group consisting of G, D, E, K, N, P, Q and Y;
-X(282)- is selected from the group consisting of V, E, G, K, P and Y;
-X(283)- is selected from the group consisting of E, G, H, K, L, P, R and Y;
-X(284)- is selected from the group consisting of V, D, E, L, N, Q, T and Y;
-X(285)- is selected from the group consisting of H, D, E, K, Q, W and Y;
-X(286)- is selected from the group consisting of N, E, G, P and Y;
-X(288)- is selected from the group consisting of K, D, E and Y;
-X(290)- is selected from the group consisting of K, D, H, L, N and W;
-X(291)- is selected from the group consisting of P, D, E, G, H, I, Q and T;
-X(292)- is selected from the group consisting of R, D, E, T and Y;
-X(293)- is selected from the group consisting of E, F, G, H, I, L, M, N, P, R, S, T, V, W and Y;
-X(294)- is selected from the group consisting of E, F, G, H, I, K, L, M, P, R, S, T, V, W and Y;
-X(295)- is selected from the group consisting of Q, D, E, F, G, H, I, M, N, P, R, S, T, V, W and Y;
-X(296)- is selected from the group consisting of F, Y, A, D, E, G, I, K, L, M, N, Q, R, S, T and V;
-X(297)- is selected from the group consisting of N, D, E, F, G, H, I, K, L, M, P, Q, R, S, T, V, W and Y;
-X(298)- is selected from the group consisting of S, E, F, H, I, K, M, Q, R, W and Y;
-X(299)- is selected from the group consisting of T, A, D, E, F, G, H, I, K, L, M, N, P, Q, R, S, V, W and Y;
-X(300)- is selected from the group consisting of F, Y, A, D, E, G, H, K, M, N, P, Q, R, S, T, V and W;
-X(301)- is selected from the group consisting of R, D, E, H and Y;
-X(302)- is selected from the group consisting of V and I;
-X(303)- is selected from the group consisting of V, D, E and Y;
-X(304)- is selected from the group consisting of S, D, H, L, N and T;
-X(305)- is selected from the group consisting of V, E, T and Y;
-X(309)- is selected from the group consisting of V and L;
-X(313)- is selected from the group consisting of W and F;
-X(317)- is selected from the group consisting of K, E and Q;
-X(318)- is selected from the group consisting of E, H, L, Q, R and Y;
-X(320)- is selected from the group consisting of K, D, F, G, H, I, L, N, P, S, T, V, W and Y;
-X(322)- is selected from the group consisting of K, D, F, G, H, I, P, S, T, V, W and Y;
-X(323)- is selected from the group consisting of V and I;
-X(324)- is selected from the group consisting of S, D, F, G, H, I, L, M, P, R, T, V, W and Y;
-X(325)- is selected from the group consisting of N, A, D, E, F, G, H, I, K, L, M, P, Q, R, S, T, V, W and Y;
-X(326)- is selected from the group consisting of K, I, L, P and T;
-X(327)- is selected from the group consisting of G, A, D, E, F, H, I, K, L, M, N, P, R, T, V, W and Y;
-X(328)- is selected from the group consisting of L, A, D, E, F, G, H, I, K, M, N, P, Q, R, S, T, V, W and Y;
-X(329)- is selected from the group consisting of P, D, E, F, G, H, I, K, L, M, N, Q, R, S, T, V, W and Y;
-X(330)- is selected from the group consisting of A, S, E, F, G, H, I, L, M, N, P, R, T, V, W and Y;
-X(331)- is selected from the group consisting of P, S, D, F, H, I, L, M, Q, R, T, V, W and Y;
-X(332)- is selected from the group consisting of I, A, D, E, F, H, K, L, M, N, P, Q, R, S, T, V, W and Y;
-X(333)- is selected from the group consisting of E, F, H, I, L, M, P, T and Y;
-X(334)- is selected from the group consisting of K, F, I, P and T;
-X(335)- is selected from the group consisting of T, D, F, G, H, I, L, M, N, P, R, S, V, W and Y;
-X(336)- is selected from the group consisting of I, E, K and Y;
-X(337)- is selected from the group consisting of S, E, H and N;
-X(339)- is selected from the group consisting of T and A;
-X(355)- is selected from the group consisting of R and Q;
-X(356)- is selected from the group consisting of E and D;
-X(358)- is selected from the group consisting of M and L;
-X(384)- is selected from the group consisting of N and S;
-X(392)- is selected from the group consisting of K and N;
-X(397)- is selected from the group consisting of M and V;
-X(409)- is selected from the group consisting of K and R;
-X(419)- is selected from the group consisting of Q and E;
-X(422)- is selected from the group consisting of V and I;
-X(435)- is selected from the group consisting of H and R;
-X(436)- is selected from the group consisting of Y and F; and
-X(445)- is selected from the group consisting of P and L.

In one variation, a first modification is selected from among S228P, S228R, substitution of the sequence RCPEPKSCDTPPPCPRCPEPKSCDTPPPCPRCPEPKSCDTPPPCPR (SEQ ID NO:20) at 228, E233P, F234L, F234V, L235A, G236-, Q268H, Q274K, N276K, F296Y, Y300F, L309V, G327A, S330A, S331P, A339T, Q355R, E356D, M358L, N384S, K392N, V397M, R409K, E419Q, V4221, H435R, Y436F, and L445P. In a further variation, a modification is selected from among, 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 2651, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N.

In a further aspect, the present application is directed to an IgG4 variant amino acid sequence including at least two modifications as compared to SEQ ID NO:13. In certain variations, a first modification is selected from among Q268H, Q274K, N276K, F296Y, Y300F, L309V, G327A, S330A, S331P, A339T, Q355R, E356D, M358L, N384S, K392N, V397M, R409K, E419Q, V4221, H435R, Y436F, and L445P. In further variations, a second modification is selected from among 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N.

In another aspect, the present application is directed to an IgG4 variant including an amino acid sequence having the formula:

C-X(227)-X(228)-CPAP-X(233)-X(234)-X(235)-X(236)-X(237)-P-X(239)-X(240)-FLFPP-
X(246)-PKDTLMIS-X(255)-TP-X(258)-V-X(260)-CVV-X(264)-DV-X(267)-X(268)-ED-X(271)-
X(272)-V-X(274)-F-X(276)-W-X(278)-VD-X(281)-V-X(283)-X(284)-HNAKT-X(290)-PR-
X(293)-E-X(295)-X(296)-NST-X(300)-RVV-X(304)-VLTV-X(309)-HQDWLNGKEYKCKV-
X(324)-N-X(326)-X(327)-X(328)-P-X(330)-X(331)-X(332)-X(333)-X(334)-TISK-X(339)-
KGQPREPQVYTLPPS-X(355)-X(356)-E-X(358)-TKNQVSLTCLVKGFYPSDIAVEWES-
X(384)-GQPENNY-X(392)-TTPP-X(397)-LDSDGSFFLYS-X(409)-LTVDKSRWQ-X(419)-GN-
X(422)-FSCSVMHEALHN-X(435)-X(436)-TQKSLSLS-X(445)-GK

wherein
-X(227)- is selected from the group consisting of P and G;
-X(228)- is selected from the group consisting of P, R, S, and the sequence

(SEQ ID NO: 20)

RCPEPKSCDTPPPCPRCPEPKSCDTPPPCPRCPEPKSCDTPPPCPR;

-X(233)- is selected from the group consisting of P and E;
-X(234)- is selected from the group consisting of V, LF, Y and I;
-X(235)- is selected from the group consisting of A, L, Y, I and D;
-X(236)- is selected from the group consisting of no amino acid, G, S and A;
-X(237)- is selected from the group consisting of G and D;
-X(239)- is selected from the group consisting of S, D, E, N, Q and T;
-X(240)- is selected from the group consisting of V, I and M;
-X(246)- is selected from the group consisting of K, H and Y;
-X(255)- is selected from the group consisting of R and Y;
-X(258)- is selected from the group consisting of E, H and Y;
-X(260)- is selected from the group consisting of T and H;
-X(264)- is selected from the group consisting of V, I, T and Y;
-X(267)- is selected from the group consisting of S, D and E;
-X(268)- is selected from the group consisting of H, Q, D and E;
-X(271)- is selected from the group consisting of P and G;
-X(272)- is selected from the group consisting of E, Y, H, R and I;
-X(274)- is selected from the group consisting of Q, K and E;
-X(276)- is selected from the group consisting of N and K;
-X(278)- is selected from the group consisting of Y and T;
-X(281)- is selected from the group consisting of G, D and E;
-X(283)- is selected from the group consisting of E, L and H;
-X(284)- is selected from the group consisting of V, E and D;
-X(290)- is selected from the group consisting of K and N;
-X(293)- is selected from the group consisting of E and R;
-X(295)- is selected from the group consisting of Q and E;
-X(296)- is selected from the group consisting of F and Y;
-X(300)- is selected from the group consisting of F and Y;
-X(304)- is selected from the group consisting of S and T;
-X(309)- is selected from the group consisting of V and L;
-X(324)- is selected from the group consisting of S, G and I;
-X(326)- is selected from the group consisting of K and T;
-X(327)- is selected from the group consisting of G, A and D;
-X(328)- is selected from the group consisting of L, A, F, I and T;
-X(330)- is selected from the group consisting of A, S, L, Y and I;
-X(331)- is selected from the group consisting of P and S;
-X(332)- is selected from the group consisting of I, D, E, N, Q and T;
-X(333)- is selected from the group consisting of E and Y;
-X(334)- is selected from the group consisting of K, F, I and T;
-X(339)- is selected from the group consisting of T and A;
-X(355)- is selected from the group consisting of R and Q;
-X(356)- is selected from the group consisting of E and D;
-X(358)- is selected from the group consisting of M and L;
-X(384)- is selected from the group consisting of N and S;
-X(392)- is selected from the group consisting of K and N;
-X(397)- is selected from the group consisting of M and V;
-X(409)- is selected from the group consisting of K and R;
-X(419)- is selected from the group consisting of Q and E;
-X(422)- is selected from the group consisting of V and I;
-X(435)- is selected from the group consisting of H and R;
-X(436)- is selected from the group consisting of Y and F; and
-X(445)- is selected from the group consisting of P and L.

In one variation, a first modification is selected from among S228P, S228R, substitution of the sequence RCPEPKSCDTPPPCPRCPEPKSCDTPPPCPRCPEPKSCDTPPPCPR (SEQ ID NO:20) at 228, E233P, F234L, F234V, L235A, G236-, Q268H, Q274K, N276K, F296Y, Y300F, L309V, G327A, S330A, S331P, A339T, Q355R, E356D, M358L, N384S, K392N, V397M, R409K, E419Q, V4221, H435R, Y436F, and L445P. In a further variation, a second modification is selected from among 227G, 234Y, 234I, 235Y, 235I, 235D, 236S, 236A, 237D, 239D, 239E, 239N, 239Q, 239T, 240I, 240M, 246H, 246Y, 255Y, 258H, 258Y, 260H, 264I, 264T, 264Y, 267D, 267E, 268D, 268E, 271G, 272Y, 272H, 272R, 272I, 274E, 278T, 281D, 281E, 283L, 283H, 284E, 284D, 290N, 293R, 295E, 304T, 324G, 324I, 326T, 327D, 328A, 328F, 328I, 328T, 330L, 330Y, 330I, 332D, 332E, 332N, 332Q, 332T, 333Y, 334F, 334I, and 334T. Alternatively, the modifications can be selected from among those beginning at position 230 until the C terminus.

In another aspect, the present application is directed to an IgG4 variant including an amino acid sequence having the formula:

ASTKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYG-X(221)-X(222)-X(223)-
X(224)-X(225)-C-X(227)-X(228)-C-X(230)-X(231)-X(232)-X(233)-X(234)-X(235)-X(236)-
X(237)-X(238)-X(239)-X(240)-X(241)-L-X(243)-X(244)-X(245)-X(246)-X(247)-K-X(249)-
TLMIS-X(255)-TP-X(258)-V-X(260)-C-X(262)-X(263)-X(264)-X(265)-X(266)-X(267)-X(268)-
X(269)-X(270)-X(271)-X(272)-X(273)-X(274)-X(275)-X(276)-W-X(278)-V-X(280)-X(281)-
X(282)-X(283)-X(284)-X(285)-X(286)-A-X(288)-T-X(290)-X(291)-X(292)-X(293)-X(294)-
X(295)-X(296)-X(297)-X(298)-X(299)-X(300)-X(301)-X(302)-X(303)-X(304)-X(305)-
LTVLHQD-X(313)-LNG-X(317)-X(318)-Y-X(320)-C-X(322)-X(323)-X(324)-X(325)-X(326)-
X(327)-X(328)-X(329)-X(330)-X(331)-X(332)-X(333)-X(334)-X(335)-X(336)-X(337)-
KAKGQPREPQVYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLGK-

wherein
-X(221)- is selected from the group consisting of no amino acid, K and Y;
-X(222)- is selected from the group consisting of no amino acid, E and Y;
-X(223)- is selected from the group consisting of no amino acid, E and K;
-X(224)- is selected from the group consisting of P and Y;
-X(225)- is selected from the group consisting of P, E, K and W;
-X(227)- is selected from the group consisting of P, E, G, K and Y;
-X(228)- is selected from the group consisting of S, E, G, K and Y;
-X(230)- is selected from the group consisting of P, A, E, G and Y;
-X(231)- is selected from the group consisting of A, E, G, K, P and Y;
-X(232)- is selected from the group consisting of P, E, G, K and Y;
-X(233)- is selected from the group consisting of E, A, D, F, G, H, I, K, L, M, N, Q, R, S, T, V, W and Y;
-X(234)- is selected from the group consisting of F, D, E, F, G, H, I, K, M, N, P, Q, R, S, T, W and Y;
-X(235)- is selected from the group consisting of L, D, F, G, H, I, K, M, N, P, Q, R, S, T, V, W and Y;
-X(236)- is selected from the group consisting of G, A, D, E, F, H, I, K, L, M, N, P, Q, R, S, T, V, W and Y;
-X(237)- is selected from the group consisting of G, D, E, F, H, I, K, L, M, N, P, Q, R, S, T, V, W and Y;
-X(238)- is selected from the group consisting of P, D, E, F, G, H, I, K, L, M, N, Q, R, S, T, V, W and Y;
-X(239)- is selected from the group consisting of S, D, E, F, G, H, I, K, L, M, N, P, Q, R, T, V, W and Y;
-X(240)- is selected from the group consisting of V, A, I, M and T;
-X(241)- is selected from the group consisting of F, D, E, L, R, S, W and Y;
-X(243)- is selected from the group consisting of F, E, H, L, Q, R, W and Y;
-X(244)- is selected from the group consisting of P and H;
-X(245)- is selected from the group consisting of P and A;
-X(246)- is selected from the group consisting of K, D, E, H and Y;
-X(247)- is selected from the group consisting of P, G and V;
-X(249)- is selected from the group consisting of D, H, Q and Y;
-X(255)- is selected from the group consisting of R, E and Y;
-X(258)- is selected from the group consisting of E, H, S and Y;
-X(260)- is selected from the group consisting of T, D, E, H and Y;
-X(262)- is selected from the group consisting of V, A, E, F, I and T;
-X(263)- is selected from the group consisting of V, A, I, M and T;
-X(264)- is selected from the group consisting of V, A, D, E, F, G, H, I, K, L, M, N, P, Q, R, S, T, W and Y;
-X(265)- is selected from the group consisting of D, F, G, H, I, K, L, M, P, Q, R, S, T, V, W and Y;
-X(266)- is selected from the group consisting of V, A, I, M and T;
-X(267)- is selected from the group consisting of S, D, E, F, H, I, K, L, M, N, P, Q, R, V, W and Y;
-X(268)- is selected from the group consisting of Q, D, E, F, G, I, K, L, M, P, R, T, V and W;
-X(269)- is selected from the group consisting of E, F, G, H, I, K, L, M, N, P, R, S, T, V, W and Y;
-X(270)- is selected from the group consisting of D, F, G, H, I, L, M, P, Q, R, S, T, W and Y;
-X(271)- is selected from the group consisting of P, A, D, E, F, G, H, I, K, L, M, N, Q, R, S, T, V, W and Y;
-X(272)- is selected from the group consisting of E, D, F, G, H, I, K, L, M, P, R, S, T, V, W and Y;
-X(273)- is selected from the group consisting of V and I;
-X(274)- is selected from the group consisting of Q, D, E, F, G, H, I, L, M, N, P, R, T, V, W and Y;
-X(275)- is selected from the group consisting of F, L, W;
-X(276)- is selected from the group consisting of N, D, E, F, G, H, I, L, M, P, R, S, T, V, W and Y;
-X(278)- is selected from the group consisting of Y, D, E, G, H, I, K, L, M, N, P, Q, R, S, T, V and W;
-X(280)- is selected from the group consisting of D, G, K, L, P and W;
-X(281)- is selected from the group consisting of G, D, E, K, N, P, Q and Y;
-X(282)- is selected from the group consisting of V, E, G, K, P and Y;
-X(283)- is selected from the group consisting of E, G, H, K, L, P, R and Y;
-X(284)- is selected from the group consisting of V, D, E, L, N, Q, T and Y;
-X(285)- is selected from the group consisting of H, D, E, K, Q, W and Y;
-X(286)- is selected from the group consisting of N, E, G, P and Y;
-X(288)- is selected from the group consisting of K, D, E and Y;
-X(290)- is selected from the group consisting of K, D, H, L, N and W;
-X(291)- is selected from the group consisting of P, D, E, G, H, I, Q and T;
-X(292)- is selected from the group consisting of R, D, E, T and Y;
-X(293)- is selected from the group consisting of E, F, G, H, I, L, M, N, P, R, S, T, V, W and Y;
-X(294)- is selected from the group consisting of E, F, G, H, I, K, L, M, P, R, S, T, V, W and Y;
-X(295)- is selected from the group consisting of Q, D, E, F, G, H, I, M, N, P, R, S, T, V, W and Y;
-X(296)- is selected from the group consisting of F, A, D, E, G, I, K, L, M, N, Q, R, S, T and V;
-X(297)- is selected from the group consisting of N, D, E, F, G, H, I, K, L, M, P, Q, R, S, T, V, W and Y;
-X(298)- is selected from the group consisting of S, E, F, H, I, K, M, Q, R, W and Y;
-X(299)- is selected from the group consisting of T, A, D, E, F, G, H, I, K, L, M, N, P, Q, R, S, V, W and Y;
-X(300)- is selected from the group consisting of Y, A, D, E, G, H, K, M, N, P, Q, R, S, T, V and W;
-X(301)- is selected from the group consisting of R, D, E, H and Y;
-X(302)- is selected from the group consisting of V and I;
-X(303)- is selected from the group consisting of V, D, E and Y;
-X(304)- is selected from the group consisting of S, D, H, L, N and T;
-X(305)- is selected from the group consisting of V, E, T and Y;
-X(313)- is selected from the group consisting of W and F;
-X(317)- is selected from the group consisting of K, E and Q;
-X(318)- is selected from the group consisting of E, H, L, Q, R and Y;
-X(320)- is selected from the group consisting of K, D, F, G, H, I, L, N, P, S, T, V, W and Y;
-X(322)- is selected from the group consisting of K, D, F, G, H, I, P, S, T, V, W and Y;
-X(323)- is selected from the group consisting of V and I;
-X(324)- is selected from the group consisting of S, D, F, G, H, I, L, M, P, R, T, V, W and Y;
-X(325)- is selected from the group consisting of N, A, D, E, F, G, H, I, K, L, M, P, Q, R, S, T, V, W and Y;
-X(326)- is selected from the group consisting of K, I, L, P and T;
-X(327)- is selected from the group consisting of G, D, E, F, H, I, K, L, M, N, P, R, T, V, W and Y;
-X(328)- is selected from the group consisting of L, A, D, E, F, G, H, I, K, M, N, P, Q, R, S, T, V, W and Y;
-X(329)- is selected from the group consisting of P, D, E, F, G, H, I, K, L, M, N, Q, R, S, T, V, W and Y;
-X(330)- is selected from the group consisting of S, E, F, G, H, I, L, M, N, P, R, T, V, W and Y;
-X(331)- is selected from the group consisting of S, D, F, H, I, L, M, Q, R, T, V, W and Y;
-X(332)- is selected from the group consisting of I, A, D, E, F, H, K, L, M, N, P, Q, R, S, T, V, W and Y;
-X(333)- is selected from the group consisting of E, F, H, I, L, M, P, T and Y;
-X(334)- is selected from the group consisting of K, F, I, P and T;
-X(335)- is selected from the group consisting of T, D, F, G, H, I, L, M, N, P, R, S, V, W and Y;
-X(336)- is selected from the group consisting of I, E, K and Y; and
-X(337)- is selected from the group consisting of S, E, H and N.

In another aspect, the present application is directed to an IgG4 variant including an amino acid sequence having the formula:

ASTKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYG-X(221)-PPC-X(227)-
SCPAPE-X(234)-X(235)-X(236)-X(237)-P-X(239)-X(240)-FLFPP-X(246)-PKDTLMIS-X(255)-
TP-X(258)-V-X(260)-CVV-X(264)-DV-X(267)-X(268)-ED-X(271)-X(272)-V-X(274)-FNW-
X(278)-VD-X(281)-V-X(283)-X(284)-HNAKT-X(290)-PR-X(293)-E-X(295)-FNSTYRVV-
X(304)-VLTVLHQDWLNGKEYKCKV-X(324)-N-X(326)-X(327)-X(328)-P-X(330)-S-X(332)-
X(333)-X(334)-
TISKAKGQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLGK

wherein
-X(221)- is selected from the group consisting of no amino acid and K;
-X(227)- is selected from the group consisting of P and G;
-X(234)- is selected from the group consisting of F, Y and I;
-X(235)- is selected from the group consisting of L, Y, I and D;
-X(236)- is selected from the group consisting of G, S and A;
-X(237)- is selected from the group consisting of G and D;
-X(239)- is selected from the group consisting of S, D, E, N, Q and T;
-X(240)- is selected from the group consisting of V, I and M;
-X(246)- is selected from the group consisting of K, H and Y;
-X(255)- is selected from the group consisting of R and Y;
-X(258)- is selected from the group consisting of E, H and Y;
-X(260)- is selected from the group consisting of T and H;
-X(264)- is selected from the group consisting of V, I, T and Y;
-X(267)- is selected from the group consisting of S, D and E;
-X(268)- is selected from the group consisting of Q, D and E;
-X(271)- is selected from the group consisting of P and G;
-X(272)- is selected from the group consisting of E, Y, H, R and I;
-X(274)- is selected from the group consisting of Q and E;
-X(278)- is selected from the group consisting of Y and T;
-X(281)- is selected from the group consisting of G, D and E;
-X(283)- is selected from the group consisting of E, L and H;
-X(284)- is selected from the group consisting of V, E and D;
-X(290)- is selected from the group consisting of K and N;
-X(293)- is selected from the group consisting of E and R;
-X(295)- is selected from the group consisting of Q and E;
-X(304)- is selected from the group consisting of S and T;
-X(324)- is selected from the group consisting of S, G and I;
-X(326)- is selected from the group consisting of K and T;
-X(327)- is selected from the group consisting of G and D;
-X(328)- is selected from the group consisting of L, A, F, I and T;
-X(330)- is selected from the group consisting of S, L, Y and I;
-X(332)- is selected from the group consisting of I, D, E, N, Q and T;
-X(333)- is selected from the group consisting of E and Y; and
-X(334)- is selected from the group consisting of K, F, I and T.

In certain variations, the variant differs from SEQ ID NO:13 by at least one amino acid.

Variations in which modifications are in 2, 3, or 4 different domains, the domains can be selected from among, for example, all IgG domains, only IgG heavy chain domains, and only hinge-CH2-CH3 domains. Alternatively, the domains can be limited to include only Fc region, or only CH2-CH3 domains.

The IgG2, IgG3, or IgG4 variants can improves binding to one or more FcγR, or enhance effector function as compared to a polypeptide having the amino acid sequence of SEQ ID NO:11, SEQ ID NO:12, or SEQ ID NO:13. In certain variations, FcγR is selected from the group consisting of human FcγRI, FcγRIIa, FcγRIIb, FcγRIIc, and FcγRIIIa. In other variations, the additionally reduces binding to human FcγRIIb. Exemplary effector function that is enhanced can be ADCC, ADCP, and CDC.

The present application is also directed to sequence including the variants described herein identified by sequence identification number.

### BRIEF DESCRIPTION OF THE DRAWINGS

FIG. 1. Antibody structure and function. Shown is a model of a full length human IgG1 antibody, modeled using a humanized Fab structure from pdb accession code 1CE1 (James et al., 1999, J Mol Biol 289:293-301) and a human IgG1 Fc structure from pdb accession code 1 DN2 (DeLano et al., 2000, Science 287:1279-1283). The flexible hinge that links the Fab and Fc regions is not shown. IgG1 is a homodimer of heterodimers, made up of two light chains and two heavy chains. The Ig domains that comprise the antibody are labeled, and include VL and CL for the light chain, and V_{H}, CH1 (Cγ1), CH2 (Cγ2), and CH3 (Cγ3) for the heavy chain. The Fc region is labeled. Binding sites for relevant proteins are labeled, including the antigen binding site in the variable region, and the binding sites for FcγRs, FcRn, C1q, and proteins A and G in the Fc region.

FIG. 2. The Fc/FcγRIIIb complex structure 1IIS. Fc is shown as a gray ribbon diagram, and FcγRIIIb is shown as a black ribbon. The N297 carbohydrate is shown as black sticks.

FIG. 3. Preferred embodiments of receptor binding profiles that include improvements to, reductions to, or no effect to the binding to various receptors, where such changes may be beneficial in certain contexts.

FIG. 4. Data for binding of IgG1 Fc variants to human FcγRI, FcγRIIa, FcγRIIb, FcγRIIc, V158 FcγRIIIa, C1q, and FcRn. The table presents for each variant the variant number (Variant), the substitution(s) of the variant, the antibody context (Context), the fold affinity relative to WT (Fold) and the confidence (Conf) in the fold affinity for binding to each Fc ligand, and the IIIa:IIb specificity ratio (IIIa:IIb) (see below). Multiple data sets were acquired for many of the variants, and all data for a given variant are grouped together. The context of the antibody indicates which antibodies have been constructed with the particular Fc variant; a=alemtuzumab, t=trastuzumab, r=rituximab, c=cetuximab, and p=PRO70769. The data provided were acquired in the context of the first antibody listed, typically alemtuzumab, although in some cases trastuzumab. An asterix (*) indicates that the data for the given Fc ligand was acquired in the context of trastuzumab. A fold (Fold) above 1 indicates an enhancement in binding affinity, and a fold below 1 indicates a reduction in binding affinity relative to the parent antibody for the given Fc ligand. Confidence values (Conf) correspond to the log confidence levels, provided from the fits of the data to a sigmoidal dose response curve. As is known in the art, a lower Conf. value indicates lower error and greater confidence in the Fold value. The lack of data for a given variant and Fc ligand indicates either that the fits to the data did not provide a meaningful value, or that the variant was not tested for that particular Fc ligand.

FIG. 5. Data for binding of IgG1 Fc variants to human V158 and F158 FcγRIIIa by AlphaScreen, binding to human V158 FcγRIIIa by SPR, and ADCC in the presence of human effector cells. The values are the fold-affinity (AlphaScreen and SPR) and fold-EC50 (ADCC) relative to WT. Context indicates the antibody variable region in which the data was acquired: a=alemtuzumab, t=trastuzumab, r=rituximab, c=cetuximab, and p=PRO70769.

FIG. 6. Non-naturally occurring modifications provided in FIG. 4, listed according to EU position. Modifications in bolded grey indicate preferred modifications.

FIG. 7. Alignment of the human IgG immunoglobulin IgG1, IgG2, IgG3, and IgG4 amino acid sequences. FIG. 7a provides the sequences of the CH1 domain and hinge region, and FIG. 7b provides the sequences of the CH2 and CH3 domains. Positions are numbered according to the EU index, and differences between IgG1 and the other immunoglobulins IgG2, IgG3, and IgG4 are shown in grey. In FIG. 7a the N-terminal end of the Fc region is indicated at EU position 226.

FIG. 8. Allotypes and isoallotypes of the human IgG1 constant chain showing the positions and the relevant amino acid substitutions (Gorman & Clark, 1990, Semin Immunol 2(6):457-66). For comparison the amino acids found in the equivalent positions in human IgG2, IgG3 and IgG4 gamma chains are also shown.

FIGS. 9a-9b. Structure of the complex of human IgG1 Fc bound to human FcγRIIIb (pdb accession code 1E4K, Sondermann et al., 2000, Nature 406:267-273), highlighting differences between IgG1 and IgG2 (FIG. 9a), and between IgG1 and IgG4 (FIG. 9b). IgG1 Fc is shown as grey ribbon, FcγRIIIb is shown as black ribbon, and IgG1 residues that differ in amino acid identity from IgG2 (FIG. 9a) and IgG4 (FIG. 9b) are shown as black sticks.

FIGS. 10a and 10b. Competition AlphaScreen™ assay showing binding of IgG1, IgG2, and IgG4 isotypes to V158 FcγRIIIa (FIG. 10a) and protein A (FIG. 10b). The variable region of the antibodies is that of the anti-Her2 antibody trastuzumab. In the presence of competitor antibody, a characteristic inhibition curve is observed as a decrease in luminescence signal. These data were normalized to the maximum and minimum luminescence signal provided by the baselines at low and high concentrations of competitor antibody respectively. The curves represent the fits of the data to a one site competition model using nonlinear regression.

FIGS. 11a-11b. Competition AlphaScreen assay showing binding of WT and variant IgG1, IgG2, and IgG4 antibodies to human V158 FcγRIIIa (FIG. 11a) and human FcγRI (FIG. 11b). The variable region of the antibodies is that of the anti-Her2 antibody trastuzumab.

FIG. 12. SPR (Surface Plasmon Resonance) data showing binding of WT and variant IgG1, IgG2, and IgG4 antibodies to human V158 FcγRIIIa. The variable region of the antibodies is that of the anti-Her2 antibody trastuzumab.

FIGS. 13a-13b. IgG1 variants with isotypic and/or novel amino acid modifications. The amino acid sequences of the human immunoglobulin isotypes IgG1, IgG2, IgG3, and IgG4 are aligned according to FIG. 7. FIG. 13a provides the sequences of the CH1 domain and hinge regions, and FIG. 13b provides the sequences of the CH2 and CH3 domains. The sequence of IgG1 is provided explicitly, and residues in the rows labeled “IgG2”, “IgG3”, and “IgG4” provide the amino acid identity at EU positions where they differ from IgG1; these modifications are isotypic modifications. Residues listed in the rows labeled “Novel” indicate novel modifications for human IgG1; these novel modifications are those indicated as preferred in FIG. 6.

FIGS. 14a-14b. IgG2 variants with isotypic and/or non-naturally occurring modifications. The amino acid sequences of the human immunoglobulin isotypes IgG2, IgG1, IgG3, and IgG4 are aligned according to FIG. 7. FIG. 14a provides the sequences of the CH1 domain and hinge regions, and FIG. 14b provides the sequences of the CH2 and CH3 domains. The sequence of IgG2 is provided explicitly, and residues in the rows labeled “IgG1”, “IgG3”, and “IgG4” provide the amino acid identity at EU positions where they differ from IgG2; these modifications are isotypic modifications. Residues listed in the rows labeled “Novel” indicate novel modifications for human IgG2; these novel modifications are those indicated as preferred in FIG. 6.

FIGS. 15a-15b. IgG3 variants with isotypic and/or non-naturally occurring modifications. The amino acid sequences of the human immunoglobulin isotypes IgG3, IgG1, IgG2, and IgG4 are aligned according to FIG. 7. FIG. 15a provides the sequences of the CH1 domain and hinge regions, and FIG. 15b provides the sequences of the CH2 and CH3 domains. The sequence of IgG3 is provided explicitly, and residues in the rows labeled “IgG1”, “IgG2”, and “IgG4” provide the amino acid identity at EU positions where they differ from IgG3; these modifications are isotypic modifications. Residues listed in the rows labeled “Novel” indicate novel modifications for human IgG3; these novel modifications are those indicated as preferred in FIG. 6.

FIGS. 16a-16b. IgG4 variants with isotypic and/or non-naturally occurring modifications. The amino acid sequences of the human immunoglobulin isotypes IgG4, IgG1, IgG2, and IgG3 are aligned according to FIG. 7. FIG. 16a provides the sequences of the CH1 domain and hinge regions, and FIG. 16b provides the sequences of the CH2 and CH3 domains. The sequence of IgG4 is provided explicitly, and residues in the rows labeled “IgG1”, “IgG2”, and “IgG3” provide the amino acid identity at EU positions where they differ from IgG4; these modifications are isotypic modifications. Residues listed in the rows labeled “Novel” indicate novel modifications for human IgG4; these novel modifications are those indicated as preferred in FIG. 6.

FIG. 17. Anti-Her2 IgG2 Variants. Novel modifications and isotypic modifications are provided for each variant, all constructed in the context of the human IgG2 isotype. The variable region (VHVL), CH1 domain (CH1), hinge region (hinge), and Fc region (Fc) are described for each variant, and the full constant region is labeled (WT IgG2, IgG2 ELLGG, or IgG(1/2) ELLGG) accordingly.

FIG. 18. Competition AlphaScreen assay showing binding of WT and IgG variant antibodies to human V158 FcγRIIIa. The variable region of the antibodies is that of the anti-Her2 antibody trastuzumab.

FIG. 19. Anti-CD30 IgG(1/2) ELLGG Variants. Novel modifications and isotypic modifications are provided for each variant. All IgG variants comprise the variable region of the anti-CD30 antibody H3.69_V2_L3.71 AC10. The variants comprise the IgG(1/2) ELLGG constant region as described in FIG. 18, and potentially one or more additional isotypic modifications and/or one or more novel modifications.

FIGS. 20a-20c. Competition AlphaScreen assay showing binding of WT and variant IgG antibodies to human V158 FcγRIIIa. IgG variants comprise the constant region of either IgG1 or IgG(1/2) ELLGG plus the indicated modifications. With the exception of I332E and S239D/I332E IgG1, all IgG variants comprise the variable region of the anti-CD30 antibody H3.69_V2_L3.71 AC10. Variants I332E IgG1 and S239D/I332E IgG1 comprise the variable region of the anti-CD30 antibody H3.69_L3.71 AC10.

FIG. 21. Data for binding of anti-CD30 IgG variants to human V158 FcγRIIIa as measured by the competition AlphaScreen. For each variant are provided the IC₅₀ (M) and Fold IC50 relative to H3.69_V2_L3.71 AC10 IgG1.

FIGS. 22a-22d. Cell-based ADCC assay of WT and variant IgGs with the variable region of the anti-CD30 antibody H3.69_V2_L3.71 AC10 or H3.69_L3.71 AC10 (133E and S239D/I332E IgG1). ADCC was measured by LDH activity using the Cytotoxicity Detection Kit (LDH, Roche Diagnostic Corporation, Indianapolis, Ind.) or the DELFIA® EuTDA-based cytotoxicity assay (Perkin Elmer, MA). For all assays, target cells were L540 Hodgkin's lymphoma cells and effector cells were human PBMCs. The figures show the dose-dependence of ADCC on antibody concentration for the indicated antibodies, normalized to the minimum and maximum fluorescence signal for each particular curve, provided by the baselines at low and high antibody concentrations respectively. The curves represent the fits of the data to a sigmoidal dose-response model using nonlinear regression.

FIG. 23. Anti-CD20 IgG(1/2) ELLGG Variants. Novel modifications and isotypic modifications are provided for each variant. All IgG variants comprise the variable region of the anti-CD20 antibody rituximab. The IgG variants comprise the IgG(1/2) ELLGG constant region and potentially one or more novel modifications.

FIG. 24. Cell-based ADCC assay of WT and variant IgGs with the variable region of the anti-CD20 antibody rituximab. ADCC was measured by LDH activity using the Cytotoxicity Detection Kit (LDH, Roche Diagnostic Corporation, Indianapolis, Ind.) according to the manufacturer's instructions, with WIL2-S lymphoma target cells and human PBMCs as effector cells.

FIG. 25. Anti-CD20 IgG(1/2) ELLGG Variants. Novel modifications and isotypic modifications are provided for each variant. All IgG variants comprise the variable region of the anti-CD20 antibody PRO70769. The variants comprise the IgG(1/2) ELLGG constant region and potentially one or more additional isotypic modifications and/or one or more novel modifications.

FIG. 26. Competition AlphaScreen assay showing binding of anti-CD20 IgG variant antibodies to human V158 FcγRIIIa. IgG variants comprise the constant region of either IgG1 or IgG(1/2) ELLGG plus the indicated modifications. All IgG variants comprise the variable region of the anti-CD20 antibody PRO70769.

FIG. 27. Cell-based ADCC assay of WT and variant IgGs with the variable region of the anti-CD20 antibody PRO70769. ADCC was measured using the DELFIA® EuTDA-based cytotoxicity assay with WIL2-S lymphoma target cells and human PBMCs as effector cells.

FIG. 28. Cell-based CDC assay of WT and variant IgGs with the variable region of the anti-CD20 antibody PRO70769. CDC assays were performed using Alamar Blue to monitor lysis of antibody -opsonized WIL2-S lymphoma cells by human serum complement (Quidel, San Diego, Calif.). The dose-dependence on antibody concentration of complement-mediated lysis is shown, normalized to the minimum and maximum fluorescence signal for each particular curve, provided by the baselines at low and high antibody concentrations respectively. The curves represent the fits of the data to a sigmoidal dose-response model using nonlinear regression.

FIGS. 29a-29h. Amino acid sequences of variable light (VL) and heavy (VH) chains used in the present invention. (SEQ ID NOS:1-8)

FIGS. 30a-30g. Amino acid sequences of constant light and heavy chains used in the present invention. (SEQ ID NOS:9-15) EU residues 233-236 are bolded in the IgG(1/2) (FIG. 30f) (SEQ ID NO:14) and IgG(1/2) ELLGG (FIG. 30g) (SEQ ID NO:15) sequences.

FIGS. 31a-31d. Amino acid sequences of IgG variant antibodies of the present invention. (SEQ ID NOS:16-19) FIGS. 31a and 31b (SEQ ID NOS:16 AND 17) provide the light and heavy chains respectively of an anti-CD2 antibody including the constant region IgG(1/2) ELLGG S239D/I332E/G327A. FIGS. 31c and 31d (SEQ ID NOS:18 AND 19) provide the light and heavy chains respectively of an anti-CD30 antibody including the constant region IgG(1/2) ELLGG S239D/I332E/G327A. EU residues 233-236, 239, 327, and 332 are bolded in the heavy chain sequences in FIGS. 31b and 31d. (SEQ ID NOS:17 and 19)

### DETAILED DESCRIPTION OF THE INVENTION

In general, therapeutic antibodies have been based on IgG1 subclass, as these generally have the best binding profiles to Fc receptors of the four IgG subclasses. However, the present invention is directed to the use of several methods that result in compositions that confer good binding profiles and/or effector function on non-IgG1 subclasses. In general, there are two types of variations that allow the use of IgG2, IgG3 and IgG4 subclasses in place of IgG1, to achieve similar, and in some cases better binding profiles to Fc receptors and/or effector function. In one general embodiment, IgG subclass modifications are made within different domains of the constant region of the heavy chain (e.g. missing the variable heavy domain; CH1-hinge-CH2-CH3). These fall into two general classes. In the first case, IgG subclass modifications, either as individual amino acid modifications or as “domain swaps”, are done. For example, some embodiments of the invention include one IgG subclass backbone with at least two domains exchanged with the same two domains of a different IgG subclass. For example, the invention provides IgG2 backbones with two different IgG1 domains. Alternatively, rather than swapping whole domains, individual amino acids are IgG subclass-modified. Thus, for example, variant IgG2 sequences contain amino acid modifications from the IgG1, IgG3 or IgG4 subclass, or combinations thereof. Similarly, variant IgG3 sequences contain amino acid modifications from the IgG1, IgG2 or IgG4 subclass, or combinations thereof. Variant IgG4 sequences contain amino acid modifications from the IgG1, IgG2 or IgG3 subclass, or combinations thereof. These changes are sometimes referred to herein as “IgG subclass modifications”. In some embodiments, these changes may be within one domain (either one or more amino acid modifications), or in the case of a plurality of modifications, between two or more domains.

A second category of variants are non-naturally occurring variants, sometimes referred to herein as “Fc variants” (it should be noted that there is positional overlap between these two groups; however, “Fc variants” do not include the IgG subclass modifications). These are amino acid modifications at particular positions that confer modified binding profiles (and/or effector function) as compared to the parent molecule but are not the specific amino acid changes seen in the different IgG subclasses.

Also included within the invention are combinations of both approaches. Thus, for example, IgG2 variants are provided that have one or more isotypic modifications, in some cases from IgG1, and one or more Fc variants as well.

### DEFINITIONS

The present application is directed IgG2, IgG3, and IgG4 variants having amino acid modifications of IgG2, IgG3, and IgG4 sequences.

In order that the application may be more completely understood, several definitions are set forth below. Such definitions are meant to encompass grammatical equivalents.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell.

By “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

By “amino acid modification” herein is meant an amino acid substitution, insertion, and/or deletion in a polypeptide sequence.

By “amino acid substitution” or “substitution” herein is meant the replacement of an amino acid at a particular position in a parent polypeptide sequence with another amino acid. For example, the substitution E272Y refers to a variant polypeptide, in this case an Fc variant, in which the glutamic acid at position 272 is replaced with tyrosine.

By “amino acid insertion” or “insertion” as used herein is meant the addition of an amino acid at a particular position in a parent polypeptide sequence. For example, -233E designates an insertion of glutamic acid at position 233.

By “amino acid deletion” or “deletion” as used herein is meant the removal of an amino acid at a particular position in a parent polypeptide sequence. For example, E233-designates the deletion of glutamic acid at position 233.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g. from about one to about ten amino acid modifications, and preferably from about one to about five amino acid modifications compared to the parent. The protein variant sequence herein will preferably possess at least about 80% homology with a parent protein sequence, and most preferably at least about 90% homology, more preferably at least about 95% homology. Variant protein can refer to the variant protein itself, compositions comprising the protein variant, or the amino acid sequence that encodes it. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification.

By “Fab” or “Fab region” as used herein is meant the polypeptides that comprises the VH, CH1, VL, and CL immunoglobulin domains. Fab may refer to this region in isolation, or this region in the context of a full length antibody or antibody fragment.

By “IgG subclass modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the IgGs comprise a glutamic acid at position 332, the substitution I332E in IgG1, IgG2, IgG3, or IgG4 is considered a non-naturally occurring modification.

By “amino acid” and “amino acid identity” as used herein is meant one of the 20 naturally occurring amino acids or any non-natural analogues that may be present at a specific, defined position.

By “effector function” as used herein is meant a biochemical event that results from the interaction of an antibody Fc region with an Fc receptor or ligand. Effector functions include but are not limited to ADCC, ADCP, and CDC.

By “effector cell” as used herein is meant a cell of the immune system that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and γδT cells, and may be from any organism including but not limited to humans, mice, rats, rabbits, and monkeys.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRs, FcγRs, FcγRs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136). Fc ligands may include undiscovered molecules that bind Fc. Particular IgG Fc ligands are Fc gamma receptors.

By “Fc gamma receptor” or “FcγR” as used herein is meant any member of the family of proteins that bind the IgG antibody Fc region and is encoded by an FcγR gene. In humans this family includes but is not limited to FcγR (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRII) (CD16), and FcγRIII-2 (CD16-2), as well as any undiscovered mouse FcγRs or FcγR isoforms or allotypes.

By “parent polypeptide” as used herein is meant an unmodified polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody.

By “position” as used herein is meant a location in the sequence of a protein. Positions may be numbered sequentially, or according to an established format, for example the EU index as in Kabat. For example, position 297 is a position in the human antibody IgG1.

By “protein” herein is meant at least two covalently attached amino acids, which includes proteins, polypeptides, oligopeptides and peptides.

By “residue” as used herein is meant a position in a protein and its associated amino acid identity. For example, Asparagine 297 (also referred to as Asn297, also referred to as N297) is a residue in the human antibody IgG1.

By “target antigen” as used herein is meant the molecule that is bound specifically by the variable region of a given antibody. A target antigen may be a protein, carbohydrate, lipid, or other chemical compound.

By “target cell” as used herein is meant a cell that expresses a target antigen.

By “variable region” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively.

By “wild type or WT” herein is meant an amino acid sequence or a nucleotide sequence that is found in nature, including allelic variations. A WT protein has an amino acid sequence or a nucleotide sequence that has not been intentionally modified.

### Antibodies

Accordingly, the present invention provides variant antibodies.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Thus, “isotype” as used herein is meant any of the subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE.

The amino-terminal portion of each chain includes a variable region of about 100 to 110 or more amino acids primarily responsible for antigen recognition. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5th edition, NIH publication, No. 91-3242, E. A. Kabat et al.).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Another type of Ig domain of the heavy chain is the hinge region. By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 220, and the IgG CH2 domain begins at residue EU position 237. Thus for IgG the antibody hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 or 230.

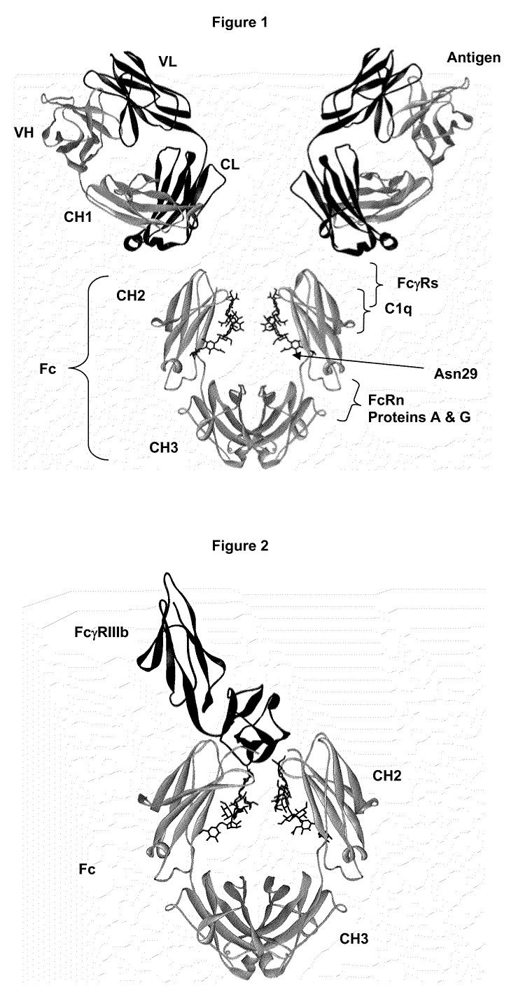

Of particular interest in the present invention are the Fc regions. By “Fc” or “Fc region”, as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, as illustrated in FIG. 1, Fc comprises immunoglobulin domains Cgamma2 and Cgamma3 (Cg2 and Cg3) and the lower hinge region between Cgamma1 (Cg1) and Cgamma2 (Cg2). Although the boundaries of the Fc region may vary, the human IgG heavy chain Fc region is usually defined to include residues C226 or P230 to its carboxyl-terminus, wherein the numbering is according to the EU index as in Kabat. Fc may refer to this region in isolation, or this region in the context of an Fc polypeptide, as described below. By “Fc polypeptide” as used herein is meant a polypeptide that comprises all or part of an Fc region. Fc polypeptides include antibodies, Fc fusions, isolated Fcs, and Fc fragments.

In some embodiments, the antibodies are full length. By “full length antibody” herein is meant the structure that constitutes the natural biological form of an antibody, including variable and constant regions, including one or more modifications as outlined herein.

Alternatively, the antibodies can be a variety of structures, including, but not limited to, antibody fragments, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each, respectively.

### Antibody Fragments

In one embodiment, the antibody is an antibody fragment. Of particular interest are antibodies that comprise Fc regions, Fc fusions, and the constant region of the heavy chain (CH1-hinge-CH2-CH3), again also including constant heavy region fusions.

Specific antibody fragments include, but are not limited to, (i) the Fab fragment consisting of VL, VH, CL and CH 1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546) which consists of a single variable, (v) isolated CDR regions, (vi) F(ab′)₂ fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883), (viii) bispecific single chain Fv dimers (PCT/US92/09965) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448). The antibody fragments may be modified. For example, the molecules may be stabilized by the incorporation of disulphide bridges linking the VH and VL domains (Reiter et al., 1996, Nature Biotech. 14:1239-1245).

### Chimeric and Humanized Antibodies

In some embodiments, the scaffold components can be a mixture from different species. As such, if the antibody is an antibody, such antibody may be a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213). The humanized antibody optimally also will comprise at least a portion of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci USA 89:4285-9, Presta et al., 1997, Cancer Res. 57(20):4593-9; Gorman et al., 1991, Proc. Natl. Acad. Sci. USA 88:4181-4185; O'Connor et al., 1998, Protein Eng 11:321-8. Humanization or other methods of reducing the immunogenicity of nonhuman antibody variable regions may include resurfacing methods, as described for example in Roguska et al., 1994, Proc. Natl. Acad. Sci. USA 91:969-973. In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759. Other humanization methods may involve the grafting of only parts of the CDRs, including but not limited to methods described in U.S. Ser. No. 09/810,502; Tan et al., 2002, J. Immunol. 169:1119-1125; De Pascalis et al., 2002, J. Immunol. 169:3076-3084.

### Bispecific Antibodies

In one embodiment, the antibodies of the invention multispecific antibody, and notably a bispecific antibody, also sometimes referred to as “diabodies”. These are antibodies that bind to two (or more) different antigens. Diabodies can be manufactured in a variety of ways known in the art (Holliger and Winter, 1993, Current Opinion Biotechnol. 4:446-449), e.g., prepared chemically or from hybrid hybridomas.

### Minibodies

In one embodiment, the antibody is a minibody. Minibodies are minimized antibody-like proteins comprising a scFv joined to a CH3 domain. Hu et al., 1996, Cancer Res. 56:3055-3061. In some cases, the scFv can be joined to the Fc region, and may include some or all of the hinge region.

### Human Antibodies

In one embodiment, the antibody is a fully human antibody with at least one modification as outlined herein. “Fully human antibody” or “complete human antibody” refers to a human antibody having the gene sequence of an antibody derived from a human chromosome with the modifications outlined herein.

### Antibody Fusions

In one embodiment, the antibodies of the invention are antibody fusion proteins (sometimes referred to herein as an “antibody conjugate”). One type of antibody fusions are Fc fusions, which join the Fc region with a conjugate partner. By “Fc fusion” as used herein is meant a protein wherein one or more polypeptides is operably linked to an Fc region. Fc fusion is herein meant to be synonymous with the terms “immunoadhesin”, “Ig fusion”, “Ig chimera”, and “receptor globulin” (sometimes with dashes) as used in the prior art (Chamow et al., 1996, Trends Biotechnol 14:52-60; Ashkenazi et al., 1997, Curr Opin Immunol 9:195-200). An Fc fusion combines the Fc region of an immunoglobulin with a fusion partner, which in general can be any protein or small molecule. Virtually any protein or small molecule may be linked to Fc to generate an Fc fusion. Protein fusion partners may include, but are not limited to, the variable region of any antibody, the target-binding region of a receptor, an adhesion molecule, a ligand, an enzyme, a cytokine, a chemokine, or some other protein or protein domain. Small molecule fusion partners may include any therapeutic agent that directs the Fc fusion to a therapeutic target. Such targets may be any molecule, preferably an extracellular receptor, that is implicated in disease.

In addition to Fc fusions, antibody fusions include the fusion of the constant region of the heavy chain with one or more fusion partners (again including the variable region of any antibody), while other antibody fusions are substantially or completely full length antibodies with fusion partners. In one embodiment, a role of the fusion partner is to mediate target binding, and thus it is functionally analogous to the variable regions of an antibody (and in fact can be). Virtually any protein or small molecule may be linked to Fc to generate an Fc fusion (or antibody fusion). Protein fusion partners may include, but are not limited to, the target-binding region of a receptor, an adhesion molecule, a ligand, an enzyme, a cytokine, a chemokine, or some other protein or protein domain. Small molecule fusion partners may include any therapeutic agent that directs the Fc fusion to a therapeutic target. Such targets may be any molecule, preferably an extracellular receptor, that is implicated in disease.

The conjugate partner can be proteinaceous or non-proteinaceous; the latter generally being generated using functional groups on the antibody and on the conjugate partner. For example linkers are known in the art; for example, homo- or hetero-bifunctional linkers as are well known (see, 1994 Pierce Chemical Company catalog, technical section on cross-linkers, pages 155-200, incorporated herein by reference).

Suitable conjugates include, but are not limited to, labels as described below, drugs and cytotoxic agents including, but not limited to, cytotoxic drugs (e.g., chemotherapeutic agents) or toxins or active fragments of such toxins. Suitable toxins and their corresponding fragments include diptheria A chain, exotoxin A chain, ricin A chain, abrin A chain, curcin, crotin, phenomycin, enomycin and the like. Cytotoxic agents also include radiochemicals made by conjugating radioisotopes to antibodies, or binding of a radionuclide to a chelating agent that has been covalently attached to the antibody. Additional embodiments utilize calicheamicin, auristatins, geldanamycin, maytansine, and duocarmycins and analogs; for the latter, see U.S. 2003/0050331, hereby incorporated by reference in its entirety.

### Covalent Modifications of Antibodies

Covalent modifications of antibodies are included within the scope of this invention, and are generally, but not always, done post-translationally. For example, several types of covalent modifications of the antibody are introduced into the molecule by reacting specific amino acid residues of the antibody with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuri benzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1M sodium cacodylate at pH 6.0

Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using 125I or 131I to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl)carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

Derivatization with bifunctional agents is useful for crosslinking antibodies to a water-insoluble support matrix or surface for use in a variety of methods, in addition to methods described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 [1983]), acetylation of the N-terminal amine, and amidation of any C-terminal carboxyl group.

### Glycosylation

Another type of covalent modification is glycosylation. In another embodiment, the IgG variants disclosed herein can be modified to include one or more engineered glycoforms. By “engineered glycoform” as used herein is meant a carbohydrate composition that is covalently attached to an IgG, wherein said carbohydrate composition differs chemically from that of a parent IgG. Engineered glycoforms may be useful for a variety of purposes, including but not limited to enhancing or reducing effector function. Engineered glycoforms may be generated by a variety of methods known in the art (Umaña et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473); (U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1); (Potelligent™ technology [Biowa, Inc., Princeton, N.J.]; GlycoMAb™ glycosylation engineering technology [GLYCART biotechnology AG, Zürich, Switzerland]). Many of these techniques are based on controlling the level of fucosylated and/or bisecting oligosaccharides that are covalently attached to the Fc region, for example by expressing an IgG in various organisms or cell lines, engineered or otherwise (for example Lec-13 CHO cells or rat hybridoma YB2/0 cells), by regulating enzymes involved in the glycosylation pathway (for example FUT8 [α1,6-fucosyltranserase] and/or β1-4-N-acetylglucosaminyltransferase III [GnTIII]), or by modifying carbohydrate(s) after the IgG has been expressed. Engineered glycoform typically refers to the different carbohydrate or oligosaccharide; thus an IgG variant, for example an antibody or Fc fusion, can include an engineered glycoform. Alternatively, engineered glycoform may refer to the IgG variant that comprises the different carbohydrate or oligosaccharide. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antibody amino acid sequence is preferably altered through changes at the DNA level, particularly by mutating the DNA encoding the target polypeptide at preselected bases such that codons are generated that will translate into the desired amino acids.

Another means of increasing the number of carbohydrate moieties on the antibody is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published Sep. 11, 1987, and in Aplin and Wriston, 1981, CRC Crit. Rev. Biochem., pp. 259-306.

Removal of carbohydrate moieties present on the starting antibody may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved by the use of a variety of endo- and exo-glycosidases as described by Thotakura et al., 1987, Meth. Enzymol. 138:350. Glycosylation at potential glycosylation sites may be prevented by the use of the compound tunicamycin as described by Duskin et al., 1982, J. Biol. Chem. 257:3105. Tunicamycin blocks the formation of protein-N-glycoside linkages.

Another type of covalent modification of the antibody comprises linking the antibody to various nonproteinaceous polymers, including, but not limited to, various polyols such as polyethylene glycol, polypropylene glycol or polyoxyalkylenes, in the manner set forth in U.S. Pat. No. 4,640,835; 4,496,689; 4,301,144; 4,670,417; 4,791,192 or 4,179,337. In addition, as is known in the art, amino acid substitutions may be made in various positions within the antibody to facilitate the addition of polymers such as PEG. See for example, U.S. Publication No. 2005/0114037, incorporated herein by reference in its entirety.

### Labeled Antibodies

In some embodiments, the covalent modification of the antibodies of the invention comprises the addition of one or more labels. In some cases, these are considered antibody fusions.

The term “labelling group” means any detectable label. In some embodiments, the labelling group is coupled to the antibody via spacer arms of various lengths to reduce potential steric hindrance. Various methods for labelling proteins are known in the art and may be used in performing the present invention.

In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.). In some embodiments, the labelling group is coupled to the antibody via spacer arms of various lengths to reduce potential steric hindrance. Various methods for labelling proteins are known in the art and may be used in performing the present invention.

Specific labels include optical dyes, including, but not limited to, chromophores, phosphors and fluorophores, with the latter being specific in many instances. Fluorophores can be either “small molecule” fluores, or proteinaceous fluores.

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland, hereby expressly incorporated by reference.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references are expressly incorporated herein by reference.

### IgG Variants

In one embodiment, the invention provides variant IgG proteins. At a minimum, IgG variants comprise an antibody fragment comprising the CH2-CH3 region of the heavy chain. In addition, suitable IgG variants comprise Fc domains (e.g. including the lower hinge region), as well as IgG variants comprising the constant region of the heavy chain (CH1-hinge-CH2-CH3) also being useful in the present invention, all of which can be fused to fusion partners.

An IgG variant includes one or more amino acid modifications relative to a parent IgG polypeptide, in some cases relative to the wild type IgG. The IgG variant can have one or more optimized properties. An IgG variant differs in amino acid sequence from its parent IgG by virtue of at least one amino acid modification. Thus IgG variants have at least one amino acid modification compared to the parent. Alternatively, the IgG variants may have more than one amino acid modification as compared to the parent, for example from about one to fifty amino acid modifications, preferably from about one to ten amino acid modifications, and most preferably from about one to about five amino acid modifications compared to the parent.

Thus the sequences of the IgG variants and those of the parent Fc polypeptide are substantially homologous. For example, the variant IgG variant sequences herein will possess about 80% homology with the parent IgG variant sequence, preferably at least about 90% homology, and most preferably at least about 95% homology. Modifications may be made genetically using molecular biology, or may be made enzymatically or chemically.

Virtually any antigen may be targeted by the IgG variants, including but not limited to proteins, subunits, domains, motifs, and/or epitopes belonging to the following list of target antigens: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxin1, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-1, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC(HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1Apo-2, DR4), TNFRSF10B (TRAIL R2 DR5, KILLER, TRICK-2A, TRICK-B), TNFRSF10C (TRAIL R3DcR1, LIT, TRID), TNFRSF10D (TRAIL R4DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3M68, TR6), TNFRSF7 (CD27), TNFRSF8 (CD30), TNFRSF9 (4-1BB CD137, ILA), TNFRSF21 (DR6), TNFRSF22 (DcTRAIL R2TNFRH2), TNFRST23 (DcTRAIL R1TNFRH1), TNFRSF25 (DR3Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factors.

Optimized IgG Variant Properties

The present application also provides IgG variants that are optimized for a variety of therapeutically relevant properties. An IgG variant that is engineered or predicted to display one or more optimized properties is herein referred to as an “optimized IgG variant”. Properties that may be optimized include but are not limited to enhanced or reduced affinity for an FcγR. In a preferred embodiment, the IgG variants are optimized to possess enhanced affinity for a human activating FcγR, preferably FcγRI, FcγRIIa, FcγRIIc, FcγRIIIa, and FcγRIIIb, most preferably FcγRIIIa. In an alternate embodiment, the IgG variants are optimized to possess reduced affinity for the human inhibitory receptor FcγRIIb. These embodiments are anticipated to provide IgG polypeptides with enhanced therapeutic properties in humans, for example enhanced effector function and greater anti-cancer potency. In an alternate embodiment, the IgG variants are optimized to have reduced or ablated affinity for a human FcγR, including but not limited to FcγRI, FcγRIIa, FcγRIIb, FcγRIIc, FcγRIIIa, and FcγRIIIb. These embodiments are anticipated to provide IgG polypeptides with enhanced therapeutic properties in humans, for example reduced effector function and reduced toxicity. In other embodiments, IgG variants provide enhanced affinity for one or more FcγRs, yet reduced affinity for one or more other FcγRs. For example, an IgG variant may have enhanced binding to FcγRIIIa, yet reduced binding to FcγRIIb. Alternately, an IgG variant may have enhanced binding to FcγRIIa and FcγRI, yet reduced binding to FcγRIIb. In yet another embodiment, an IgG variant may have enhanced affinity for FcγRIIb, yet reduced affinity to one or more activating FcγRs.

Preferred embodiments comprise optimization of binding to a human FcγR, however in alternate embodiments the IgG variants possess enhanced or reduced affinity for FcγRs from nonhuman organisms, including but not limited to rodents and non-human primates. IgG variants that are optimized for binding to a nonhuman FcγR may find use in experimentation. For example, mouse models are available for a variety of diseases that enable testing of properties such as efficacy, toxicity, and pharmacokinetics for a given drug candidate. As is known in the art, cancer cells can be grafted or injected into mice to mimic a human cancer, a process referred to as xenografting. Testing of IgG variants that comprise IgG variants that are optimized for one or more mouse FcγRs, may provide valuable information with regard to the efficacy of the protein, its mechanism of action, and the like. The IgG variants may also be optimized for enhanced functionality and/or solution properties in aglycosylated form. In a preferred embodiment, the aglycosylated IgG variants bind an Fc ligand with greater affinity than the aglycosylated form of the parent IgG variant. The Fc ligands include but are not limited to FcγRs, C1q, FcRn, and proteins A and G, and may be from any source including but not limited to human, mouse, rat, rabbit, or monkey, preferably human. In an alternately preferred embodiment, the IgG variants are optimized to be more stable and/or more soluble than the aglycosylated form of the parent IgG variant.

IgG variants can include modifications that modulate interaction with Fc ligands other than FcγRs, including but not limited to complement proteins, FcRn, and Fc receptor homologs (FcRHs). FcRHs include but are not limited to FcRH1, FcRH2, FcRH3, FcRH4, FcRH5, and FcRH6 (Davis et al., 2002, Immunol. Reviews 190:123-136).

Preferably, the Fc ligand specificity of the IgG variant will determine its therapeutic utility. The utility of a given IgG variant for therapeutic purposes will depend on the epitope or form of the Target antigen and the disease or indication being treated. For some targets and indications, enhanced FcγR-mediated effector functions may be preferable. This may be particularly favorable for anti-cancer IgG variants. Thus IgG variants may be used that comprise IgG variants that provide enhanced affinity for activating FcγRs and/or reduced affinity for inhibitory FcγRs. For some targets and indications, it may be further beneficial to utilize IgG variants that provide differential selectivity for different activating FcγRs; for example, in some cases enhanced binding to FcγRIIa and FcγRIIIa may be desired, but not FcγR, whereas in other cases, enhanced binding only to FcγRIIa may be preferred. For certain targets and indications, it may be preferable to utilize IgG variants that enhance both FcγR-mediated and complement-mediated effector functions, whereas for other cases it may be advantageous to utilize IgG variants that enhance either FcγR-mediated or complement-mediated effector functions. For some targets or cancer indications, it may be advantageous to reduce or ablate one or more effector functions, for example by knocking out binding to C1q, one or more FcγR's, FcRn, or one or more other Fc ligands. For other targets and indications, it may be preferable to utilize IgG variants that provide enhanced binding to the inhibitory FcγRIIb, yet WT level, reduced, or ablated binding to activating FcγRs. This may be particularly useful, for example, when the goal of an IgG variant is to inhibit inflammation or auto-immune disease, or modulate the immune system in some way.

Clearly an important parameter that determines the most beneficial selectivity of a given IgG variant to treat a given disease is the context of the IgG variant, that is what type of IgG variant is being used. Thus the Fc ligand selectivity or specificity of a given IgG variant will provide different properties depending on whether it composes an antibody, Fc fusion, or an IgG variants with a coupled fusion or conjugate partner. For example, toxin, radionucleotide, or other conjugates may be less toxic to normal cells if the IgG variant that comprises them has reduced or ablated binding to one or more Fc ligands. As another example, in order to inhibit inflammation or auto-immune disease, it may be preferable to utilize an IgG variant with enhanced affinity for activating FcγRs, such as to bind these FcγRs and prevent their activation. Conversely, an IgG variant that comprises two or more Fc regions with enhanced FcγRIIb affinity may co-engage this receptor on the surface of immune cells, thereby inhibiting proliferation of these cells. Whereas in some cases an IgG variants may engage its target antigen on one cell type yet engage FcγRs on separate cells from the target antigen, in other cases it may be advantageous to engage FcγRs on the surface of the same cells as the target antigen. For example, if an antibody targets an antigen on a cell that also expresses one or more FcγRs, it may be beneficial to utilize an IgG variant that enhances or reduces binding to the FcγRs on the surface of that cell. This may be the case, for example when the IgG variant is being used as an anti-cancer agent, and co-engagement of target antigen and FcγR on the surface of the same cell promote signaling events within the cell that result in growth inhibition, apoptosis, or other anti-proliferative effect. Alternatively, antigen and FcγR co-engagement on the same cell may be advantageous when the IgG variant is being used to modulate the immune system in some way, wherein co-engagement of target antigen and FcγR provides some proliferative or anti-proliferative effect. Likewise, IgG variants that comprise two or more Fc regions may benefit from IgG variants that modulate FcγR selectivity or specificity to co-engage FcγRs on the surface of the same cell.

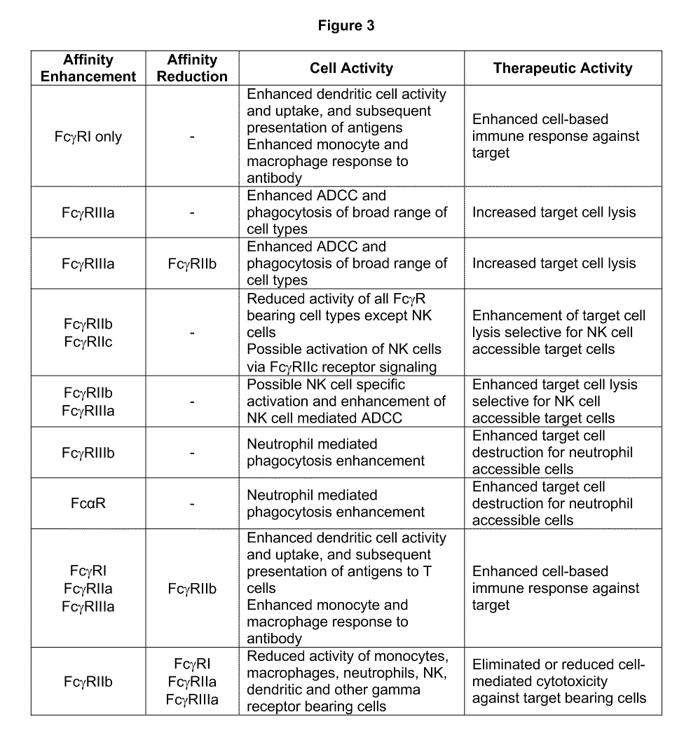

The Fc ligand specificity of the IgG variants can be modulated to create different effector function profiles that may be suited for particular target antigens, indications, or patient populations. FIG. 3 describes several preferred embodiments of receptor binding profiles that include improvements to, reductions to or no effect to the binding to various receptors, where such changes may be beneficial in certain contexts. The receptor binding profiles in the table could be varied by degree of increase or decrease to the specified receptors. Additionally, the binding changes specified could be in the context of additional binding changes to other receptors such as C1q or FcRn, for example by combining with ablation of binding to C1q to shut off complement activation, or by combining with enhanced binding to C1q to increase complement activation. Other embodiments with other receptor binding profiles are possible, the listed receptor binding profiles are exemplary.

The presence of different polymorphic forms of FcγRs provides yet another parameter that impacts the therapeutic utility of the IgG variants. Whereas the specificity and selectivity of a given IgG variant for the different classes of FcγRs significantly affects the capacity of an IgG variant to target a given antigen for treatment of a given disease, the specificity or selectivity of an IgG variant for different polymorphic forms of these receptors may in part determine which research or pre-clinical experiments may be appropriate for testing, and ultimately which patient populations may or may not respond to treatment. Thus the specificity or selectivity of IgG variants to Fc ligand polymorphisms, including but not limited to FcγR, C1q, FcRn, and FcRH polymorphisms, may be used to guide the selection of valid research and pre-clinical experiments, clinical trial design, patient selection, dosing dependence, and/or other aspects concerning clinical trials.

Modification may be made to improve the IgG stability, solubility, function, or clinical use. In a preferred embodiment, the IgG variants can include modifications to reduce immunogenicity in humans. In a most preferred embodiment, the immunogenicity of an IgG variant is reduced using a method described in U.S. Ser. No. 11/004,590, filed Dec. 3, 2004, entitled “Methods of Generating Variant Proteins with Increased Host String Content and Compositions Thereof”. In alternate embodiments, the IgG variants are humanized (Clark, 2000, Immunol Today 21:397-402).

The IgG variants can include modifications that reduce immunogenicity. Modifications to reduce immunogenicity can include modifications that reduce binding of processed peptides derived from the parent sequence to MHC proteins. For example, amino acid modifications would be engineered such that there are no or a minimal number of immune epitopes that are predicted to bind, with high affinity, to any prevalent MHC alleles. Several methods of identifying MHC-binding epitopes in protein sequences are known in the art and may be used to score epitopes in an IgG variant. See for example WO 98/52976; WO 02/079232; WO 00/3317; U.S. Ser. No. 09/903,378; U.S. Ser. No. 10/039,170; U.S. Ser. No. 60/222,697; U.S. Ser. No. 10/754,296; PCT WO 01/21823; and PCT WO 02/00165; Mallios, 1999, Bioinformatics 15: 432-439; Mallios, 2001, Bioinformatics 17: 942-948; Sturniolo et al., 1999, Nature Biotech. 17: 555-561; WO 98/59244; WO 02/069232; WO 02/77187; Marshall et al., 1995, J. Immunol. 154: 5927-5933; and Hammer et al., 1994, J. Exp. Med. 180: 2353-2358. Sequence-based information can be used to determine a binding score for a given peptide—MHC interaction (see for example Mallios, 1999, Bioinformatics 15: 432-439; Mallios, 2001, Bioinformatics 17: p 942-948; Sturniolo et. al., 1999, Nature Biotech. 17: 555-561).

Fusion Partners

The IgG variants can be linked to one or more fusion partners. In one alternate embodiment, the IgG variant is conjugated or operably linked to another therapeutic compound. The therapeutic compound may be a cytotoxic agent, a chemotherapeutic agent, a toxin, a radioisotope, a cytokine, or other therapeutically active agent. The IgG may be linked to one of a variety of nonproteinaceous polymers, e.g., polyethylene glycol, polypropylene glycol, polyoxyalkylenes, or copolymers of polyethylene glycol and polypropylene glycol.

Engineering IgG Variants

The IgG variants can be based on human IgG sequences, and thus human IgG sequences are used as the “base” sequences against which other sequences are compared, including but not limited to sequences from other organisms, for example rodent and primate sequences. IgG variants may also comprise sequences from other immunoglobulin classes such as IgA, IgE, IgGD, IgGM, and the like. It is contemplated that, although the IgG variants are engineered in the context of one parent IgG, the variants may be engineered in or “transferred” to the context of another, second parent IgG. This is done by determining the “equivalent” or “corresponding” residues and substitutions between the first and second IgG, typically based on sequence or structural homology between the sequences of the two IgGs. In order to establish homology, the amino acid sequence of a first IgG outlined herein is directly compared to the sequence of a second IgG. After aligning the sequences, using one or more of the homology alignment programs known in the art (for example using conserved residues as between species), allowing for necessary insertions and deletions in order to maintain alignment (i.e., avoiding the elimination of conserved residues through arbitrary deletion and insertion), the residues equivalent to particular amino acids in the primary sequence of the first IgG variant are defined. Alignment of conserved residues preferably should conserve 100% of such residues. However, alignment of greater than 75% or as little as 50% of conserved residues is also adequate to define equivalent residues. Equivalent residues may also be defined by determining structural homology between a first and second IgG that is at the level of tertiary structure for IgGs whose structures have been determined. In this case, equivalent residues are defined as those for which the atomic coordinates of two or more of the main chain atoms of a particular amino acid residue of the parent or precursor (N on N, CA on CA, C on C and O on O) are within 0.13 nm and preferably 0.1 nm after alignment. Alignment is achieved after the best model has been oriented and positioned to give the maximum overlap of atomic coordinates of non-hydrogen protein atoms of the proteins. Regardless of how equivalent or corresponding residues are determined, and regardless of the identity of the parent IgG in which the IgGs are made, what is meant to be conveyed is that the IgG variants discovered by can be engineered into any second parent IgG that has significant sequence or structural homology with the IgG variant. Thus for example, if a variant antibody is generated wherein the parent antibody is human IgG1, by using the methods described above or other methods for determining equivalent residues, the variant antibody may be engineered in another IgG1 parent antibody that binds a different antigen, a human IgG2 parent antibody, a human IgA parent antibody, a mouse IgG2a or IgG2b parent antibody, and the like. Again, as described above, the context of the parent IgG variant does not affect the ability to transfer the IgG variants to other parent IgGs.

Methods for engineering, producing, and screening IgG variants are provided. The described methods are not meant to constrain to any particular application or theory of operation. Rather, the provided methods are meant to illustrate generally that one or more IgG variants may be engineered, produced, and screened experimentally to obtain IgG variants with optimized effector function. A variety of methods are described for designing, producing, and testing antibody and protein variants in U.S. Ser. No. 10/754,296, and U.S. Ser. No. 10/672,280, which are herein expressly incorporated by reference.

A variety of protein engineering methods may be used to design IgG variants with optimized effector function. In one embodiment, a structure-based engineering method may be used, wherein available structural information is used to guide substitutions. In a preferred embodiment, a computational screening method may be used, wherein substitutions are designed based on their energetic fitness in computational calculations. See for example U.S. Ser. No. 10/754,296 and U.S. Ser. No. 10/672,280, and references cited therein.

An alignment of sequences may be used to guide substitutions at the identified positions. One skilled in the art will appreciate that the use of sequence information may curb the introduction of substitutions that are potentially deleterious to protein structure. The source of the sequences may vary widely, and include one or more of the known databases, including but not limited to the Kabat database (Northwestern University); Johnson & Wu, 2001, Nucleic Acids Res. 29:205-206; Johnson & Wu, 2000, Nucleic Acids Res. 28:214-218), the IMGT database (IMGT, the international ImMunoGeneTics Information System®; Lefranc et al., 1999, Nucleic Acids Res. 27:209-212; Ruiz et al., 2000 Nucleic Acids Res. 28:219-221; Lefranc et al., 2001, Nucleic Acids Res. 29:207-209; Lefranc et al., 2003, Nucleic Acids Res. 31:307-310), and VBASE. Antibody sequence information can be obtained, compiled, and/or generated from sequence alignments of germline sequences or sequences of naturally occurring antibodies from any organism, including but not limited to mammals. One skilled in the art will appreciate that the use of sequences that are human or substantially human may further have the advantage of being less immunogenic when administered to a human. Other databases which are more general nucleic acid or protein databases, i.e. not particular to antibodies, include but are not limited to SwissProt, GenBank Entrez, and EMBL Nucleotide Sequence Database. Aligned sequences can include VH, VL, CH, and/or CL sequences. There are numerous sequence-based alignment programs and methods known in the art, and all of these find use in for generation of sequence alignments.

Alternatively, random or semi-random mutagenesis methods may be used to make amino acid modifications at the desired positions. In these cases positions are chosen randomly, or amino acid changes are made using simplistic rules. For example all residues may be mutated to alanine, referred to as alanine scanning. Such methods may be coupled with more sophisticated engineering approaches that employ selection methods to screen higher levels of sequence diversity. As is well known in the art, there are a variety of selection technologies that may be used for such approaches, including, for example, display technologies such as phage display, ribosome display, cell surface display, and the like, as described below.

Methods for production and screening of IgG variants are well known in the art. General methods for antibody molecular biology, expression, purification, and screening are described in Antibody Engineering, edited by Duebel & Kontermann, Springer-Verlag, Heidelberg, 2001; and Hayhurst & Georgiou, 2001, Curr Opin Chem Biol 5:683-689; Maynard & Georgiou, 2000, Annu Rev Biomed Eng 2:339-76. Also see the methods described in U.S. Ser. No. 10/754,296, filed on Mar. 3, 2003, U.S. Ser. No. 10/672,280, filed Sep. 29, 2003, and U.S. Ser. No. 10/822,231, filed Mar. 26, 2004.

Making IgG Variants

The IgG variants can be made by any method known in the art. In one embodiment, the IgG variant sequences are used to create nucleic acids that encode the member sequences, and that may then be cloned into host cells, expressed and assayed, if desired. These practices are carried out using well-known procedures, and a variety of methods that may find use in are described in Molecular Cloning—A Laboratory Manual, 3^{rd} Ed. (Maniatis, Cold Spring Harbor Laboratory Press, New York, 2001), and Current Protocols in Molecular Biology (John Wiley & Sons). The nucleic acids that encode the IgG variants may be incorporated into an expression vector in order to express the protein. Expression vectors typically include a protein operably linked, that is placed in a functional relationship, with control or regulatory sequences, selectable markers, any fusion partners, and/or additional elements. The IgG variants may be produced by culturing a host cell transformed with nucleic acid, preferably an expression vector, containing nucleic acid encoding the IgG variants, under the appropriate conditions to induce or cause expression of the protein. A wide variety of appropriate host cells may be used, including but not limited to mammalian cells, bacteria, insect cells, and yeast. For example, a variety of cell lines that may find use in are described in the ATCC cell line catalog, available from the American Type Culture Collection. The methods of introducing exogenous nucleic acid into host cells are well known in the art, and will vary with the host cell used.

In a preferred embodiment, IgG variants are purified or isolated after expression. Antibodies may be isolated or purified in a variety of ways known to those skilled in the art. Standard purification methods include chromatographic techniques, electrophoretic, immunological, precipitation, dialysis, filtration, concentration, and chromatofocusing techniques. As is well known in the art, a variety of natural proteins bind antibodies, for example bacterial proteins A, G, and L, and these proteins may find use in for purification. Purification can often be enabled by a particular fusion partner. For example, proteins may be purified using glutathione resin if a GST fusion is employed, Ni⁺² affinity chromatography if a His-tag is employed, or immobilized anti-flag antibody if a flag-tag is used. For general guidance in suitable purification techniques, see Antibody Purification: Principles and Practice, 3^{rd} Ed., Scopes, Springer-Verlag, NY, 1994.

Screening IgG Variants

IgG variants may be screened using a variety of methods, including but not limited to those that use in vitro assays, in vivo and cell-based assays, and selection technologies. Automation and high-throughput screening technologies may be utilized in the screening procedures. Screening may employ the use of a fusion partner or label, for example an immune label, isotopic label, or small molecule label such as a fluorescent or colorimetric dye.

In a preferred embodiment, the functional and/or biophysical properties of IgG variants are screened in an in vitro assay. In a preferred embodiment, the protein is screened for functionality, for example its ability to catalyze a reaction or its binding affinity to its target. Binding assays can be carried out using a variety of methods known in the art, including but not limited to FRET (Fluorescence Resonance Energy Transfer) and BRET (Bioluminescence Resonance Energy Transfer)-based assays, AlphaScreen™ (Amplified Luminescent Proximity Homogeneous Assay), Scintillation Proximity Assay, ELISA (Enzyme-Linked Immunosorbent Assay), SPR (Surface Plasmon Resonance, also known as BIACORE®), isothermal titration calorimetry, differential scanning calorimetry, gel electrophoresis, and chromatography including gel filtration. These and other methods may take advantage of some fusion partner or label. Assays may employ a variety of detection methods including but not limited to chromogenic, fluorescent, luminescent, or isotopic labels. The biophysical properties of proteins, for example stability and solubility, may be screened using a variety of methods known in the art. Protein stability may be determined by measuring the thermodynamic equilibrium between folded and unfolded states. For example, IgG variants may be unfolded using chemical denaturant, heat, or pH, and this transition may be monitored using methods including but not limited to circular dichroism spectroscopy, fluorescence spectroscopy, absorbance spectroscopy, NMR spectroscopy, calorimetry, and proteolysis. As will be appreciated by those skilled in the art, the kinetic parameters of the folding and unfolding transitions may also be monitored using these and other techniques. The solubility and overall structural integrity of a IgG variant may be quantitatively or qualitatively determined using a wide range of methods that are known in the art. Methods which may find use in for characterizing the biophysical properties of IgG variants include gel electrophoresis, chromatography such as size exclusion chromatography and reversed-phase high performance liquid chromatography, mass spectrometry, ultraviolet absorbance spectroscopy, fluorescence spectroscopy, circular dichroism spectroscopy, isothermal titration calorimetry, differential scanning calorimetry, analytical ultra-centrifugation, dynamic light scattering, proteolysis, and cross-linking, turbidity measurement, filter retardation assays, immunological assays, fluorescent dye binding assays, protein-staining assays, microscopy, and detection of aggregates via ELISA or other binding assay. Structural analysis employing X-ray crystallographic techniques and NMR spectroscopy may also find use.

As is known in the art, a subset of screening methods are those that select for favorable members of a library. The methods are herein referred to as “selection methods”, and these methods find use in for screening IgG variants. When protein libraries are screened using a selection method, only those members of a library that are favorable, that is which meet some selection criteria, are propagated, isolated, and/or observed. As will be appreciated, because only the most fit variants are observed, such methods enable the screening of libraries that are larger than those screenable by methods that assay the fitness of library members individually. Selection is enabled by any method, technique, or fusion partner that links, covalently or noncovalently, the phenotype of a protein with its genotype, that is the function of a protein with the nucleic acid that encodes it. For example the use of phage display as a selection method is enabled by the fusion of library members to the gene III protein. In this way, selection or isolation of IgG variants that meet some criteria, for example binding affinity to the protein's target, also selects for or isolates the nucleic acid that encodes it. Once isolated, the gene or genes encoding Fc variants may then be amplified. This process of isolation and amplification, referred to as panning, may be repeated, allowing favorable IgG variants in the library to be enriched. Nucleic acid sequencing of the attached nucleic acid ultimately allows for gene identification.

A variety of selection methods are known in the art that may find use in for screening protein libraries. These include but are not limited to phage display (Phage display of peptides and proteins: a laboratory manual, Kay et al., 1996, Academic Press, San Diego, Calif., 1996; Lowman et al., 1991, Biochemistry 30:10832-10838; Smith, 1985, Science 228:1315-1317) and its derivatives such as selective phage infection (Malmborg et al., 1997, J Mol Biol 273:544-551), selectively infective phage (Krebber et al., 1997, J Mol Biol 268:619-630), and delayed infectivity panning (Benhar et al., 2000, J Mol Biol 301:893-904), cell surface display (Witrrup, 2001, Curr Opin Biotechnol, 12:395-399) such as display on bacteria (Georgiou et al., 1997, Nat Biotechnol 15:29-34; Georgiou et al., 1993, Trends Biotechnol 11:6-10; Lee et al., 2000, Nat Biotechnol 18:645-648; Jun et al., 1998, Nat Biotechnol 16:576-80), yeast (Boder & Wittrup, 2000, Methods Enzymol 328:430-44; Boder & Wittrup, 1997, Nat Biotechnol 15:553-557), and mammalian cells (Whitehorn et al., 1995, Bio/technology 13:1215-1219), as well as in vitro display technologies (Amstutz et al., 2001, Curr Opin Biotechnol 12:400-405) such as polysome display (Mattheakis et al., 1994, Proc Natl Acad Sci USA 91:9022-9026), ribosome display (Hanes et al., 1997, Proc Natl Acad Sci USA 94:4937-4942), mRNA display (Roberts & Szostak, 1997, Proc Natl Acad Sci USA 94:12297-12302; Nemoto et al., 1997, FEBS Lett 414:405-408), and ribosome-inactivation display system (Zhou et al., 2002, J Am Chem Soc 124, 538-543).

Other selection methods that may find use in include methods that do not rely on display, such as in vivo methods including but not limited to periplasmic expression and cytometric screening (Chen et al., 2001, Nat Biotechnol 19:537-542), the protein fragment complementation assay (Johnsson & Varshavsky, 1994, Proc Natl Acad Sci USA 91:10340-10344; Pelletier et al., 1998, Proc Natl Acad Sci USA 95:12141-12146), and the yeast two hybrid screen (Fields & Song, 1989, Nature 340:245-246) used in selection mode (Visintin et al., 1999, Proc Natl Acad Sci USA 96:11723-11728). In an alternate embodiment, selection is enabled by a fusion partner that binds to a specific sequence on the expression vector, thus linking covalently or noncovalently the fusion partner and associated Fc variant library member with the nucleic acid that encodes them. For example, U.S. Ser. No. 09/642,574; U.S. Ser. No. 10/080,376; U.S. Ser. No. 09/792,630; U.S. Ser. No. 10/023,208; U.S. Ser. No. 09/792,626; U.S. Ser. No. 10/082,671; U.S. Ser. No. 09/953,351; U.S. Ser. No. 10/097,100; U.S. Ser. No. 60/366,658; PCT WO 00/22906; PCT WO 01/49058; PCT WO 02/04852; PCT WO 02/04853; PCT WO 02/08023; PCT WO 01/28702; and PCT WO 02/07466 describe such a fusion partner and technique that may find use in. In an alternative embodiment, in vivo selection can occur if expression of the protein imparts some growth, reproduction, or survival advantage to the cell.

A subset of selection methods referred to as “directed evolution” methods are those that include the mating or breading of favorable sequences during selection, sometimes with the incorporation of new mutations. As will be appreciated by those skilled in the art, directed evolution methods can facilitate identification of the most favorable sequences in a library, and can increase the diversity of sequences that are screened. A variety of directed evolution methods are known in the art that may find use in for screening IgG variants, including but not limited to DNA shuffling (PCT WO 00/42561 A3; PCT WO 01/70947 A3), exon shuffling (U.S. Pat. No. 6,365,377; Kolkman & Stemmer, 2001, Nat Biotechnol 19:423-428), family shuffling (Crameri et al., 1998, Nature 391:288-291; U.S. Pat. No. 6,376,246), RACHITT™ (Coco et al., 2001, Nat Biotechnol 19:354-359; PCT WO 02/06469), STEP and random priming of in vitro recombination (Zhao et al., 1998, Nat Biotechnol 16:258-261; Shao et al., 1998, Nucleic Acids Res 26:681-683), exonuclease mediated gene assembly (U.S. Pat. No. 6,352,842; U.S. Pat. No. 6,361,974), Gene Site Saturation Mutagenesis™ (U.S. Pat. No. 6,358,709), Gene Reassembly™ (U.S. Pat. No. 6,358,709), SCRATCHY (Lutz et al., 2001, Proc Natl Acad Sci USA 98:11248-11253), DNA fragmentation methods (Kikuchi et al., Gene 236:159-167), single-stranded DNA shuffling (Kikuchi et al., 2000, Gene 243:133-137), and AMEsystem™ directed evolution protein engineering technology (Applied Molecular Evolution) (U.S. Pat. No. 5,824,514; U.S. Pat. No. 5,817,483; U.S. Pat. No. 5,814,476; U.S. Pat. No. 5,763,192; U.S. Pat. No. 5,723,323).

In a preferred embodiment, IgG variants are screened using one or more cell-based or in vivo assays. For such assays, purified or unpurified proteins are typically added exogenously such that cells are exposed to individual variants or pools of variants belonging to a library. These assays are typically, but not always, based on the function of the IgG; that is, the ability of the IgG to bind to its target and mediate some biochemical event, for example effector function, ligand/receptor binding inhibition, apoptosis, and the like. Such assays often involve monitoring the response of cells to the IgG, for example cell survival, cell death, change in cellular morphology, or transcriptional activation such as cellular expression of a natural gene or reporter gene. For example, such assays may measure the ability of IgG variants to elicit ADCC, ADCP, or CDC. For some assays additional cells or components, that is in addition to the target cells, may need to be added, for example example serum complement, or effector cells such as peripheral blood monocytes (PBMCs), NK cells, macrophages, and the like. Such additional cells may be from any organism, preferably humans, mice, rat, rabbit, and monkey. Antibodies may cause apoptosis of certain cell lines expressing the target, or they may mediate attack on target cells by immune cells which have been added to the assay. Methods for monitoring cell death or viability are known in the art, and include the use of dyes, immunochemical, cytochemical, and radioactive reagents. For example, caspase staining assays may enable apoptosis to be measured, and uptake or release of radioactive substrates or fluorescent dyes such as alamar blue may enable cell growth or activation to be monitored. In a preferred embodiment, the DELFIA® EuTDA-based cytotoxicity assay (Perkin Elmer, MA) is used. Alternatively, dead or damaged target cells may be monitoried by measuring the release of one or more natural intracellular proteins, for example lactate dehydrogenase. Transcriptional activation may also serve as a method for assaying function in cell-based assays. In this case, response may be monitored by assaying for natural genes or proteins which may be upregulated, for example the release of certain interleukins may be measured, or alternatively readout may be via a reporter construct. Cell-based assays may also involve the measure of morphological changes of cells as a response to the presence of a protein. Cell types for such assays may be prokaryotic or eukaryotic, and a variety of cell lines that are known in the art may be employed. Alternatively, cell-based screens are performed using cells that have been transformed or transfected with nucleic acids encoding the variants. That is, IgG variants are not added exogenously to the cells. For example, in one embodiment, the cell-based screen utilizes cell surface display. A fusion partner can be employed that enables display of IgG variants on the surface of cells (Witrrup, 2001, Curr Opin Biotechnol, 12:395-399).

In a preferred embodiment, the immunogenicity of the IgG variants is determined experimentally using one or more cell-based assays. Several methods can be used for experimental confirmation of epitopes. In a preferred embodiment, ex vivo T-cell activation assays are used to experimentally quantitate immunogenicity. In this method, antigen presenting cells and naïve T cells from matched donors are challenged with a peptide or whole protein of interest one or more times. Then, T cell activation can be detected using a number of methods, for example by monitoring production of cytokines or measuring uptake of tritiated thymidine. In the most preferred embodiment, interferon gamma production is monitored using Elispot assays (Schmittel et. al., 2000, J. Immunol. Meth., 24: 17-24).

The biological properties of the IgG variants may be characterized in cell, tissue, and whole organism experiments. As is known in the art, drugs are often tested in animals, including but not limited to mice, rats, rabbits, dogs, cats, pigs, and monkeys, in order to measure a drug's efficacy for treatment against a disease or disease model, or to measure a drug's pharmacokinetics, toxicity, and other properties. The animals may be referred to as disease models. Therapeutics are often tested in mice, including but not limited to nude mice, SCID mice, xenograft mice, and transgenic mice (including knockins and knockouts). Such experimentation may provide meaningful data for determination of the potential of the protein to be used as a therapeutic. Any organism, preferably mammals, may be used for testing. For example because of their genetic similarity to humans, monkeys can be suitable therapeutic models, and thus may be used to test the efficacy, toxicity, pharmacokinetics, or other property of the IgGs. Tests of the in humans are ultimately required for approval as drugs, and thus of course these experiments are contemplated. Thus the IgGs may be tested in humans to determine their therapeutic efficacy, toxicity, immunogenicity, pharmacokinetics, and/or other clinical properties.

Methods of Using IgG Variants

The IgG variants may find use in a wide range of products. In one embodiment the IgG variant is a therapeutic, a diagnostic, or a research reagent, preferably a therapeutic. The IgG variant may find use in an antibody composition that is monoclonal or polyclonal. In a preferred embodiment, the IgG variants are used to kill target cells that bear the target antigen, for example cancer cells. In an alternate embodiment, the IgG variants are used to block, antagonize, or agonize the target antigen, for example for antagonizing a cytokine or cytokine receptor. In an alternately preferred embodiment, the IgG variants are used to block, antagonize, or agonize the target antigen and kill the target cells that bear the target antigen.

The IgG variants may be used for various therapeutic purposes. In a preferred embodiment, an antibody comprising the IgG variant is administered to a patient to treat an antibody-related disorder. A “patient” for the purposes includes both humans and other animals, preferably mammals and most preferably humans. By “antibody related disorder” or “antibody responsive disorder” or “condition” or “disease” herein are meant a disorder that may be ameliorated by the administration of a pharmaceutical composition comprising an IgG variant. Antibody related disorders include but are not limited to autoimmune diseases, immunological diseases, infectious diseases, inflammatory diseases, neurological diseases, and oncological and neoplastic diseases including cancer. By “cancer” and “cancerous” herein refer to or describe the physiological condition in mammals that is typically characterized by unregulated cell growth. Examples of cancer include but are not limited to carcinoma, lymphoma, blastoma, sarcoma (including liposarcoma), neuroendocrine tumors, mesothelioma, schwanoma, meningioma, adenocarcinoma, melanoma, and leukemia and lymphoid malignancies.

In one embodiment, an IgG variant is the only therapeutically active agent administered to a patient. Alternatively, the IgG variant is administered in combination with one or more other therapeutic agents, including but not limited to cytotoxic agents, chemotherapeutic agents, cytokines, growth inhibitory agents, anti-hormonal agents, kinase inhibitors, anti-angiogenic agents, cardioprotectants, or other therapeutic agents. The IgG variants may be administered concomitantly with one or more other therapeutic regimens. For example, an IgG variant may be administered to the patient along with chemotherapy, radiation therapy, or both chemotherapy and radiation therapy. In one embodiment, the IgG variant may be administered in conjunction with one or more antibodies, which may or may not be an IgG variant. In accordance with another embodiment, the IgG variant and one or more other anti-cancer therapies are employed to treat cancer cells ex vivo. It is contemplated that such ex vivo treatment may be useful in bone marrow transplantation and particularly, autologous bone marrow transplantation. It is of course contemplated that the IgG variants can be employed in combination with still other therapeutic techniques such as surgery.

A variety of other therapeutic agents may find use for administration with the IgG variants. In one embodiment, the IgG is administered with an anti-angiogenic agent. By “anti-angiogenic agent” as used herein is meant a compound that blocks, or interferes to some degree, the development of blood vessels. The anti-angiogenic factor may, for instance, be a small molecule or a protein, for example an antibody, Fc fusion, or cytokine, that binds to a growth factor or growth factor receptor involved in promoting angiogenesis. The preferred anti-angiogenic factor herein is an antibody that binds to Vascular Endothelial Growth Factor (VEGF). In an alternate embodiment, the IgG is administered with a therapeutic agent that induces or enhances adaptive immune response, for example an antibody that targets CTLA-4. In an alternate embodiment, the IgG is administered with a tyrosine kinase inhibitor. By “tyrosine kinase inhibitor” as used herein is meant a molecule that inhibits to some extent tyrosine kinase activity of a tyrosine kinase. In an alternate embodiment, the IgG variants are administered with a cytokine. By “cytokine” as used herein is meant a generic term for proteins released by one cell population that act on another cell as intercellular mediators.

Pharmaceutical compositions are contemplated wherein an IgG variant and one or more therapeutically active agents are formulated. Formulations of the IgG variants are prepared for storage by mixing the IgG having the desired degree of purity with optional pharmaceutically acceptable carriers, excipients or stabilizers (Remington's Pharmaceutical Sciences 16th edition, Osol, A. Ed., 1980), in the form of lyophilized formulations or aqueous solutions. The formulations to be used for in vivo administration are preferably sterile. This is readily accomplished by filtration through sterile filtration membranes or other methods. The IgG variants and other therapeutically active agents disclosed herein may also be formulated as immunoliposomes, and/or entrapped in microcapsules

The concentration of the therapeutically active IgG variant in the formulation may vary from about 0.1 to 100 weight %. In a preferred embodiment, the concentration of the IgG is in the range of 0.003 to 1.0 molar. In order to treat a patient, a therapeutically effective dose of the IgG variant may be administered. By “therapeutically effective dose” herein is meant a dose that produces the effects for which it is administered. The exact dose will depend on the purpose of the treatment, and will be ascertainable by one skilled in the art using known techniques. Dosages may range from 0.01 to 100 mg/kg of body weight or greater, for example 0.1, 1, 10, or 50 mg/kg of body weight, with 1 to 10 mg/kg being preferred. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

Administration of the pharmaceutical composition comprising an IgG variant, preferably in the form of a sterile aqueous solution, may be done in a variety of ways, including, but not limited to, orally, subcutaneously, intravenously, intranasally, intraotically, transdermally, topically (e.g., gels, salves, lotions, creams, etc.), intraperitoneally, intramuscularly, intrapulmonary (e.g., AERx® inhalable technology commercially available from Aradigm, or Inhance™ pulmonary delivery system commercially available from Inhale Therapeutics), vaginally, parenterally, rectally, or intraocularly.

### EXAMPLES

Examples are provided below to illustrate the present invention. These examples are not meant to constrain the present invention to any particular application or theory of operation. For all positions discussed in the present invention, numbering is according to the EU index as in Kabat (Kabat et al., 1991, Sequences of Proteins of Immunological Interest, 5th Ed., United States Public Health Service, National Institutes of Health, Bethesda). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

### Example 1

### Non-Naturally Occurring Modifications

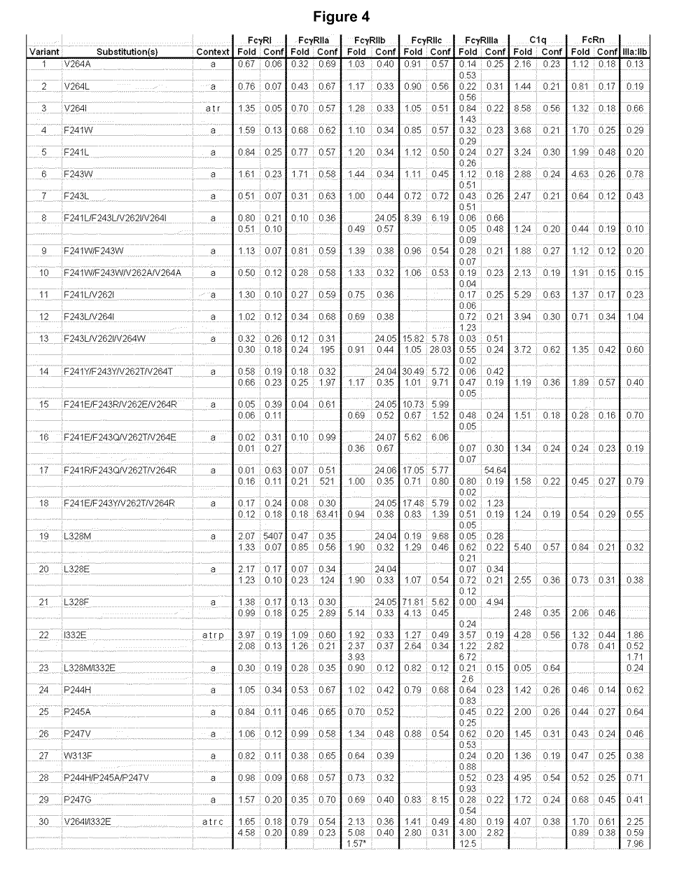

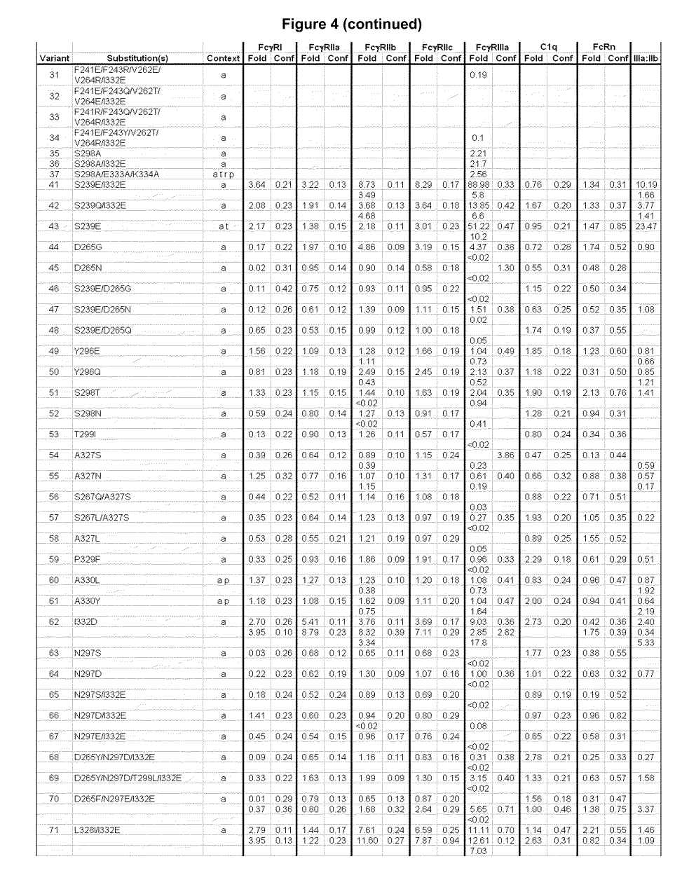

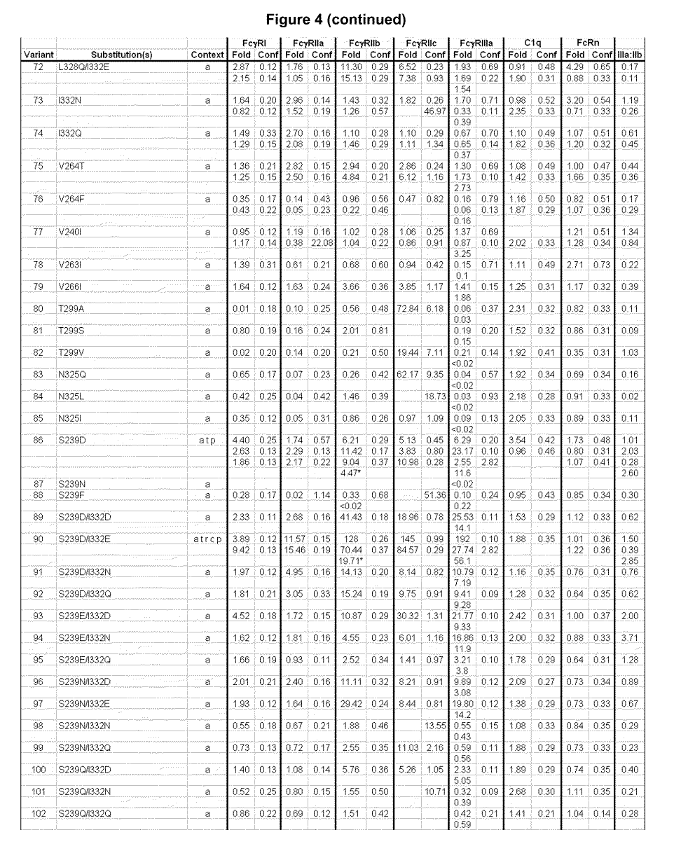

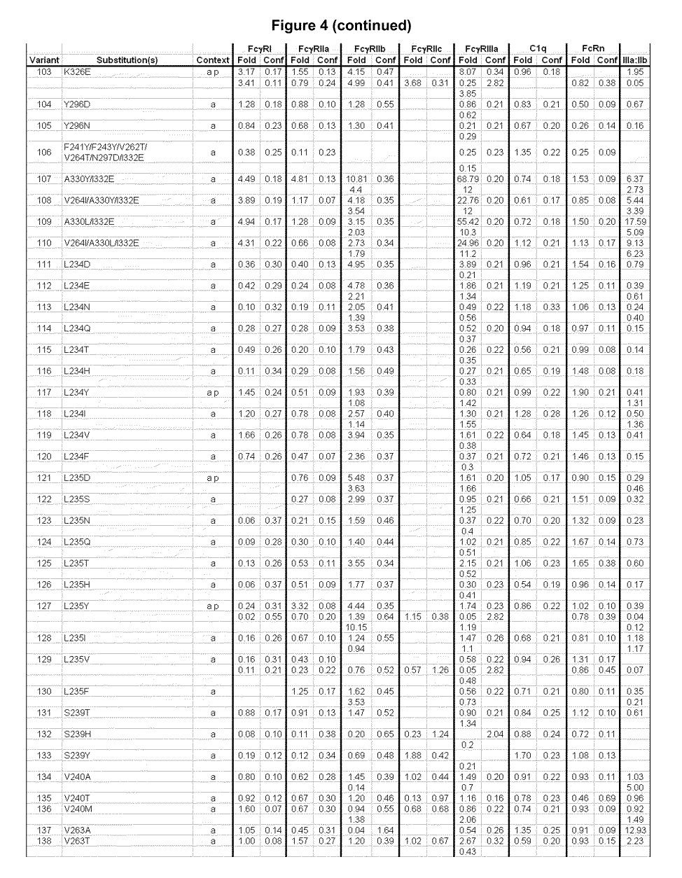

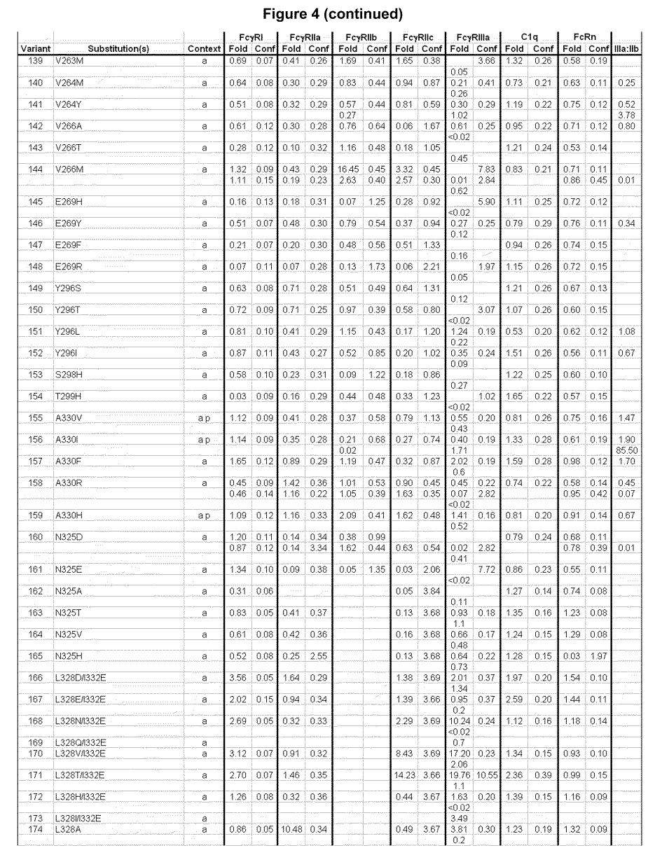

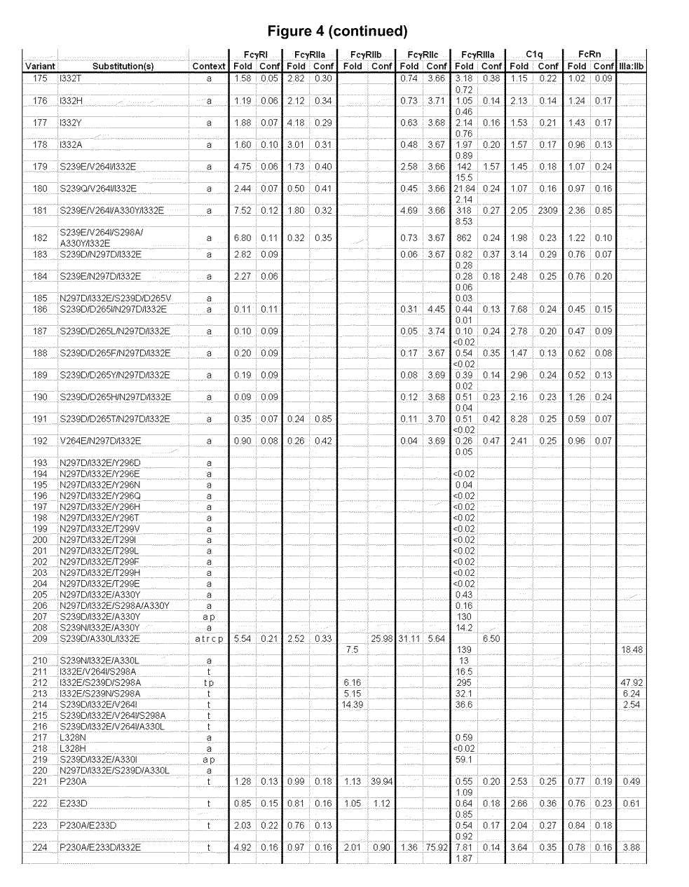

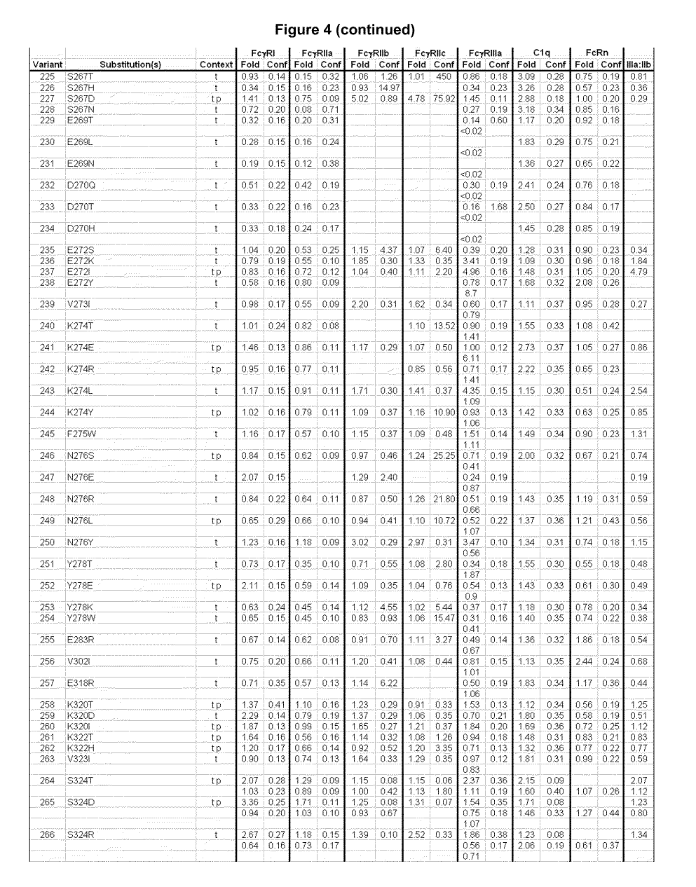

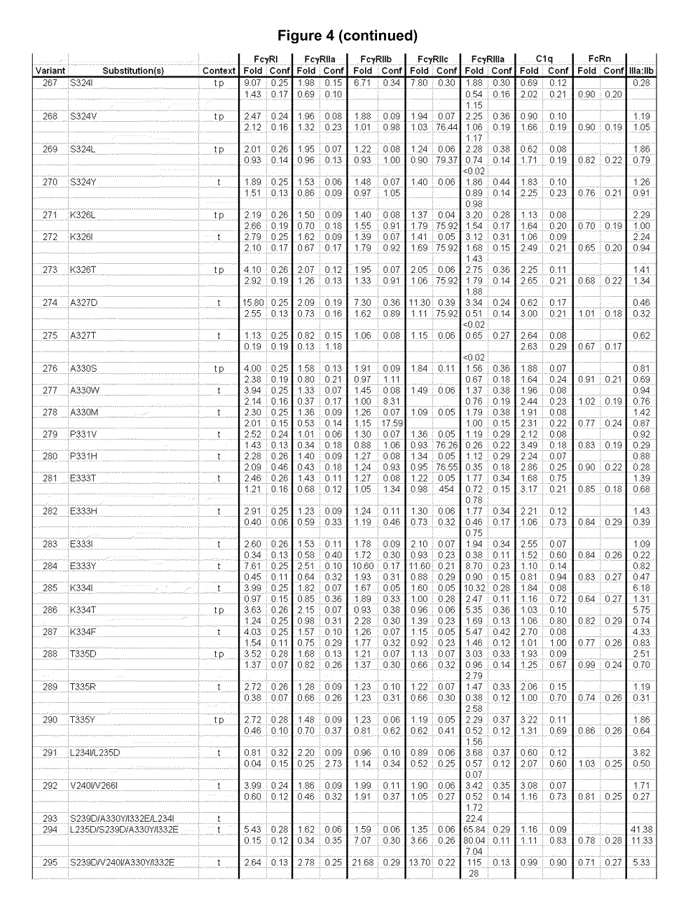

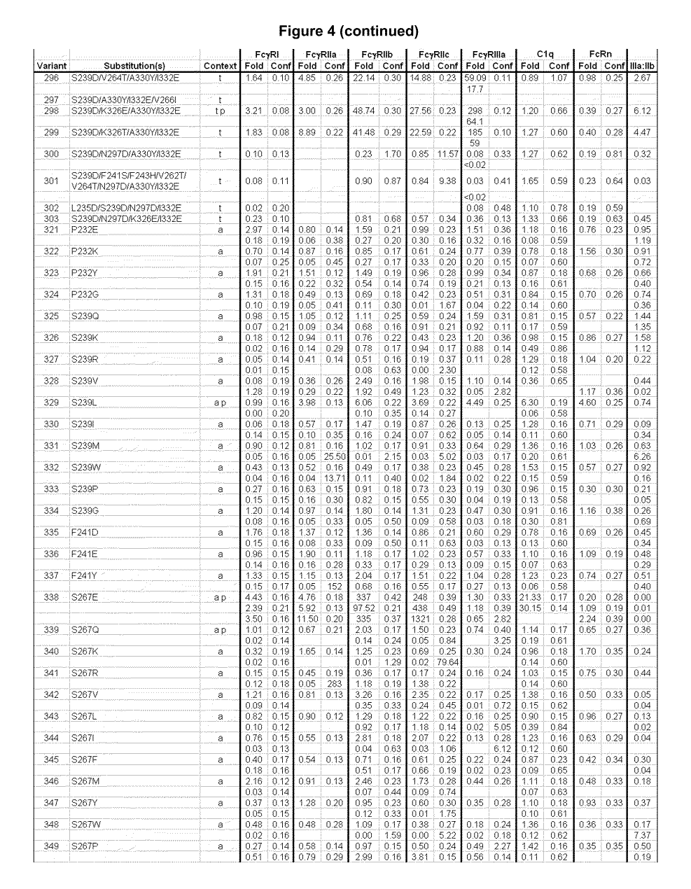

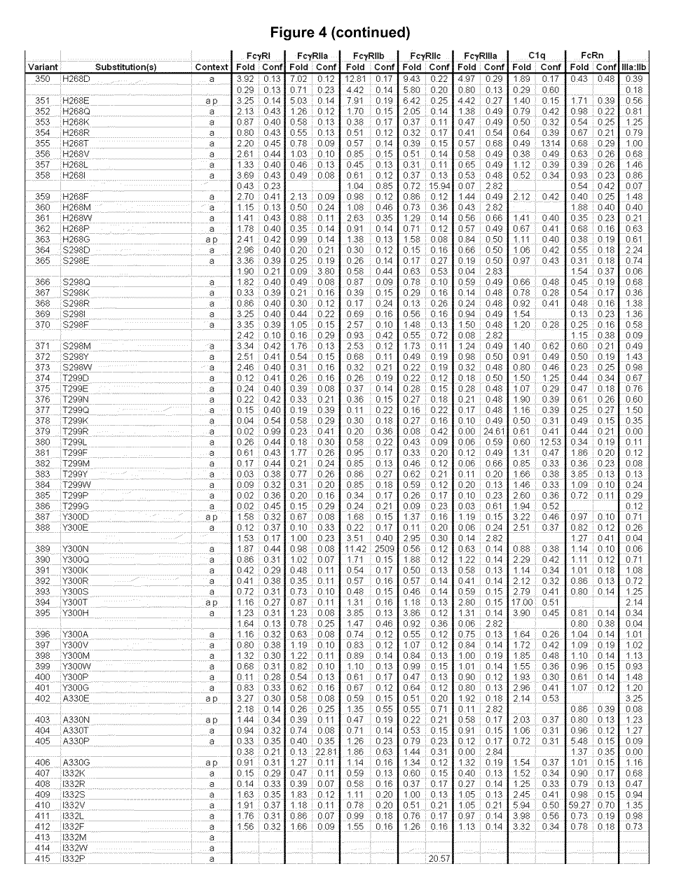

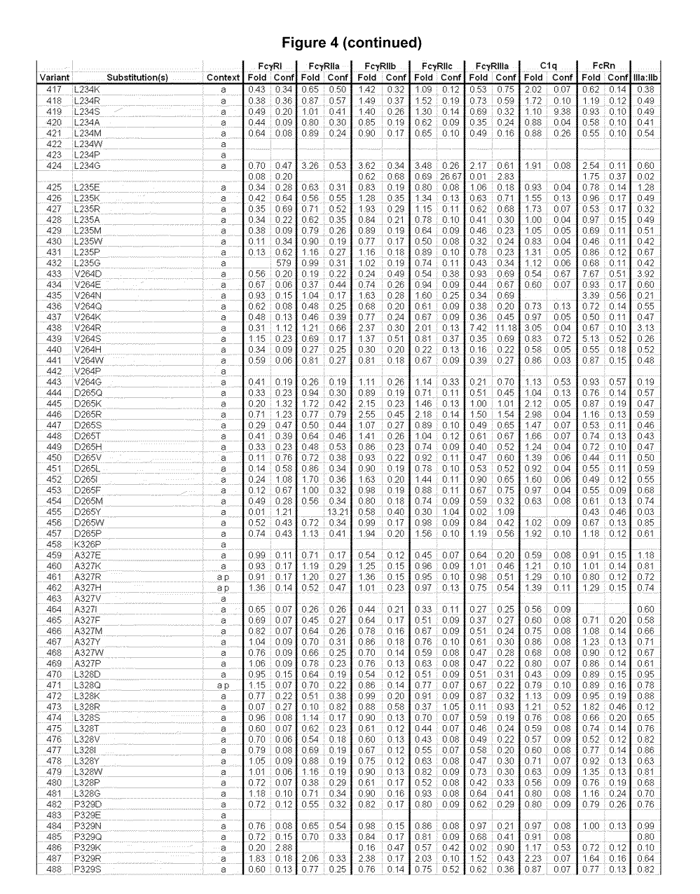

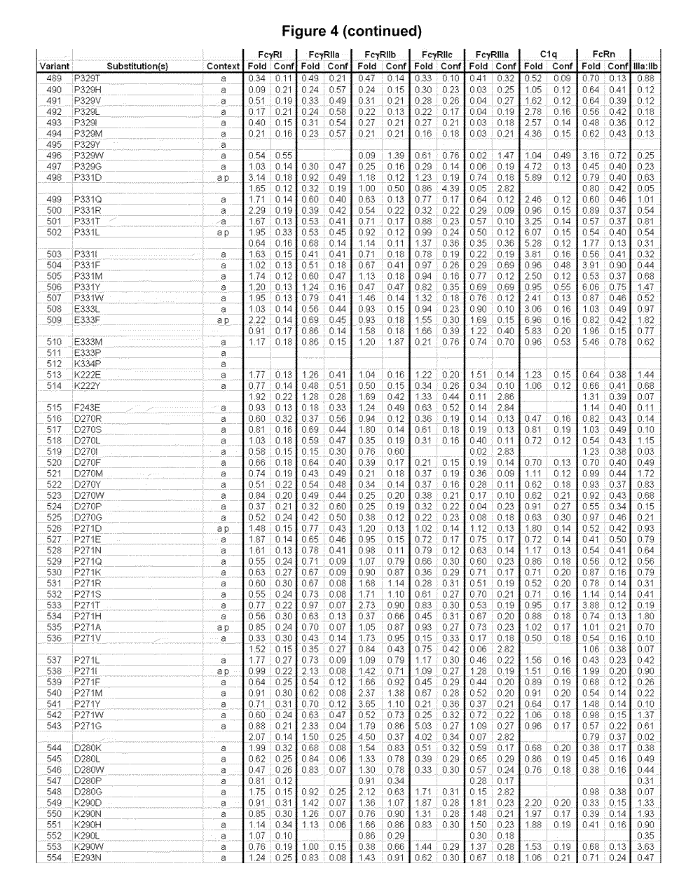

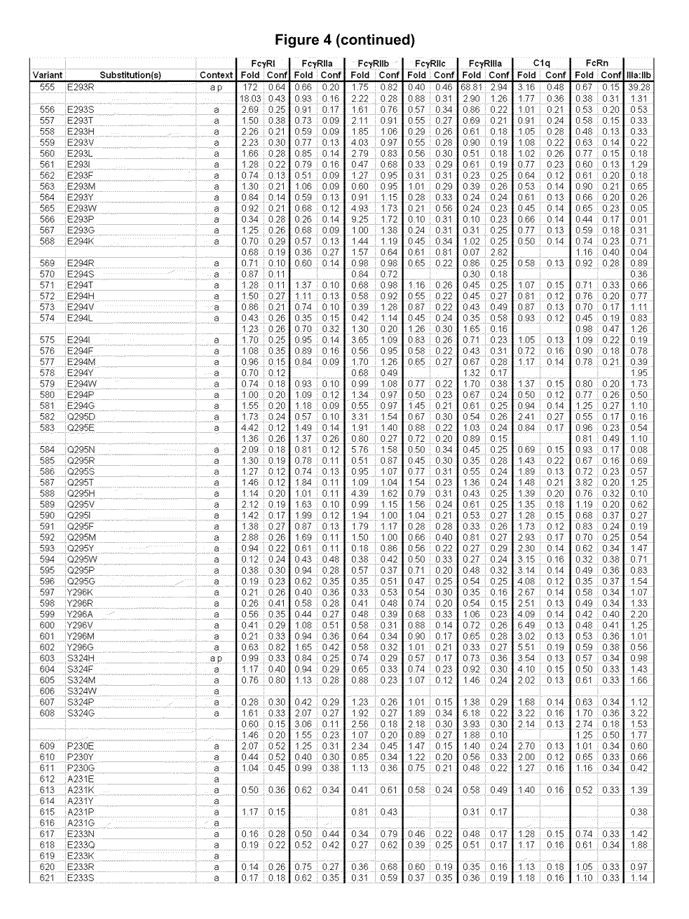

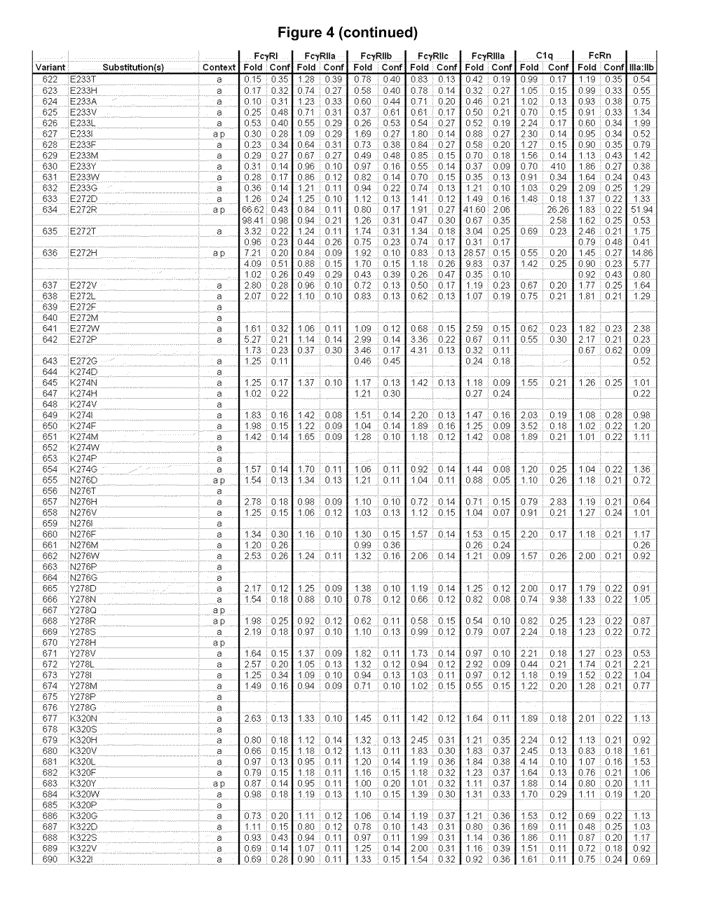

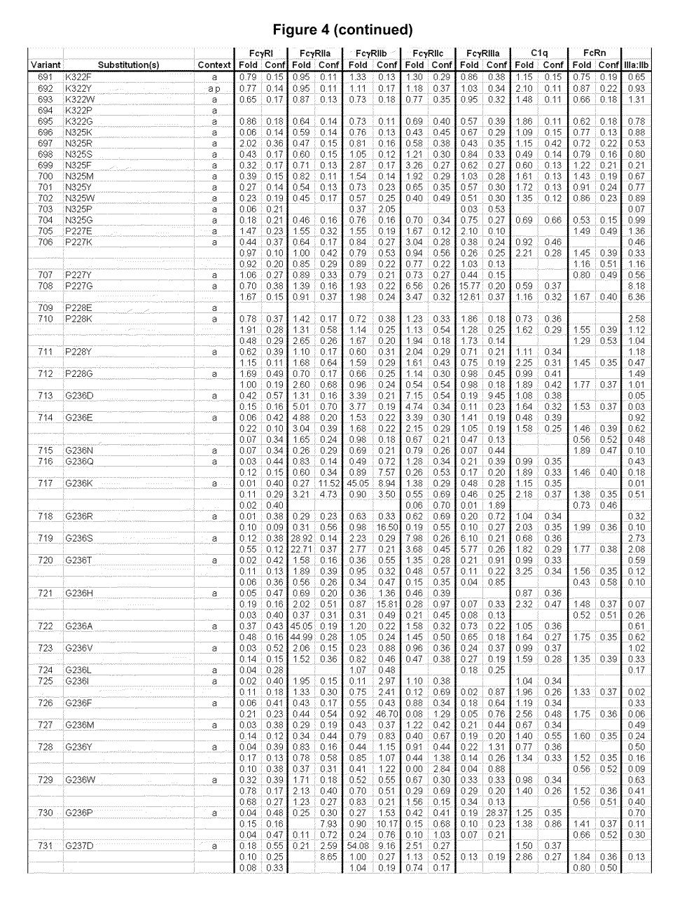

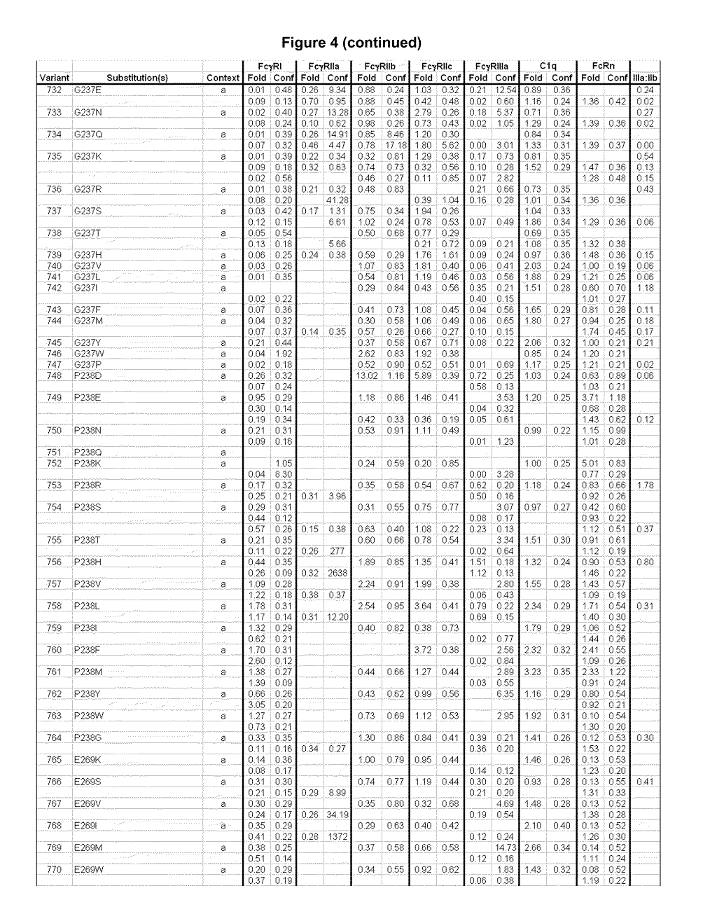

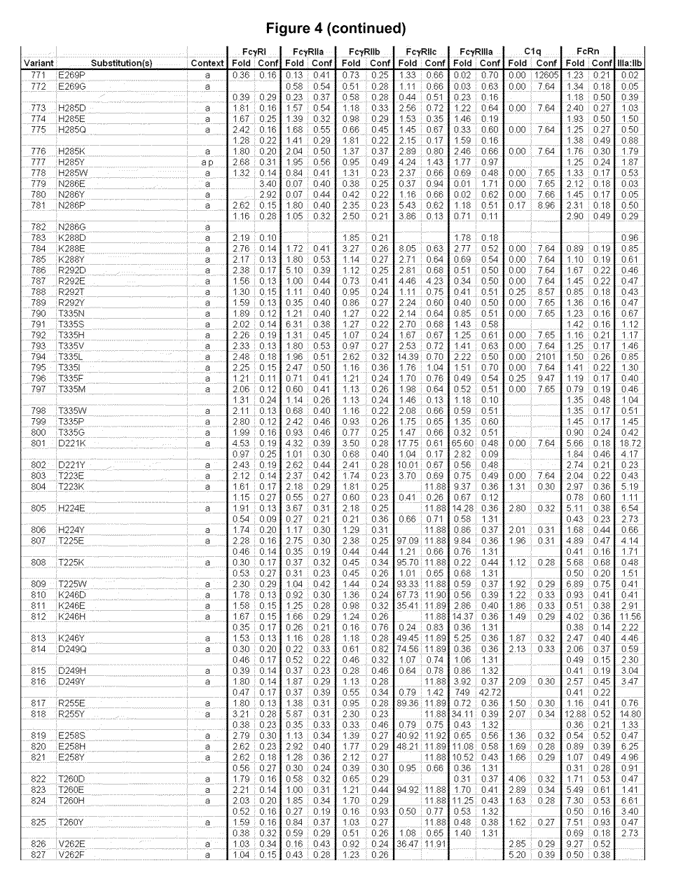

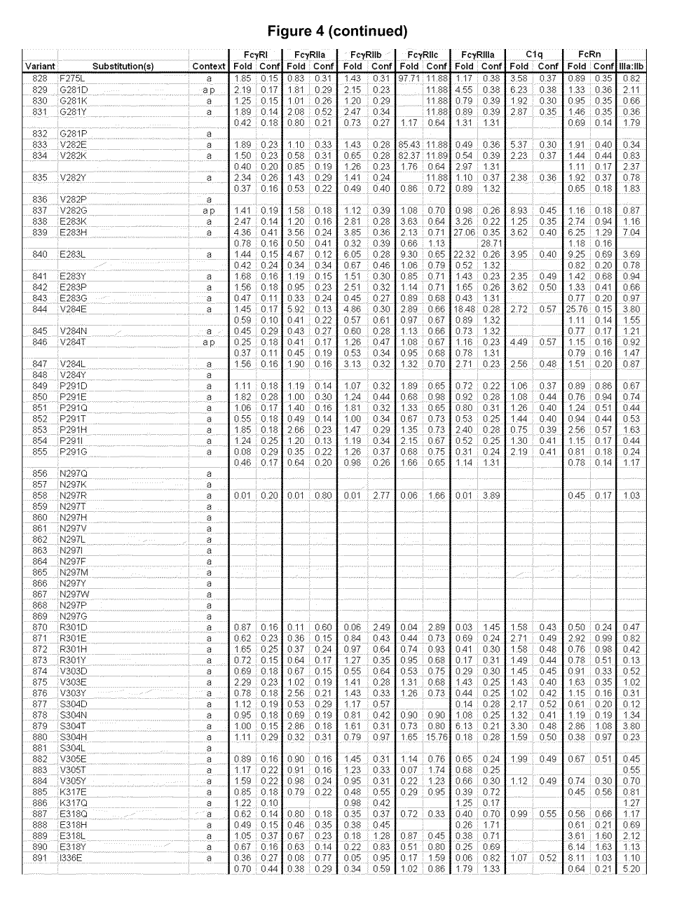

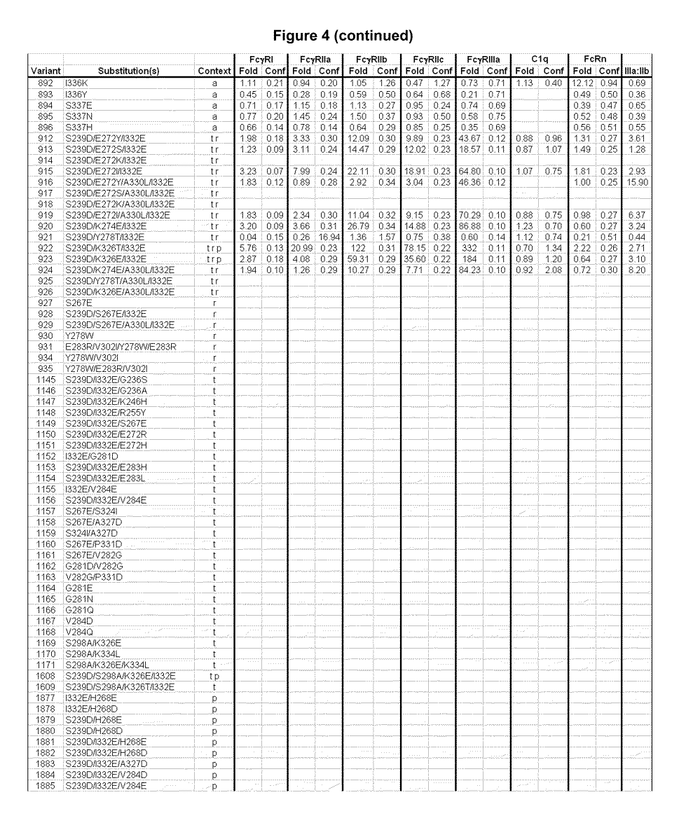

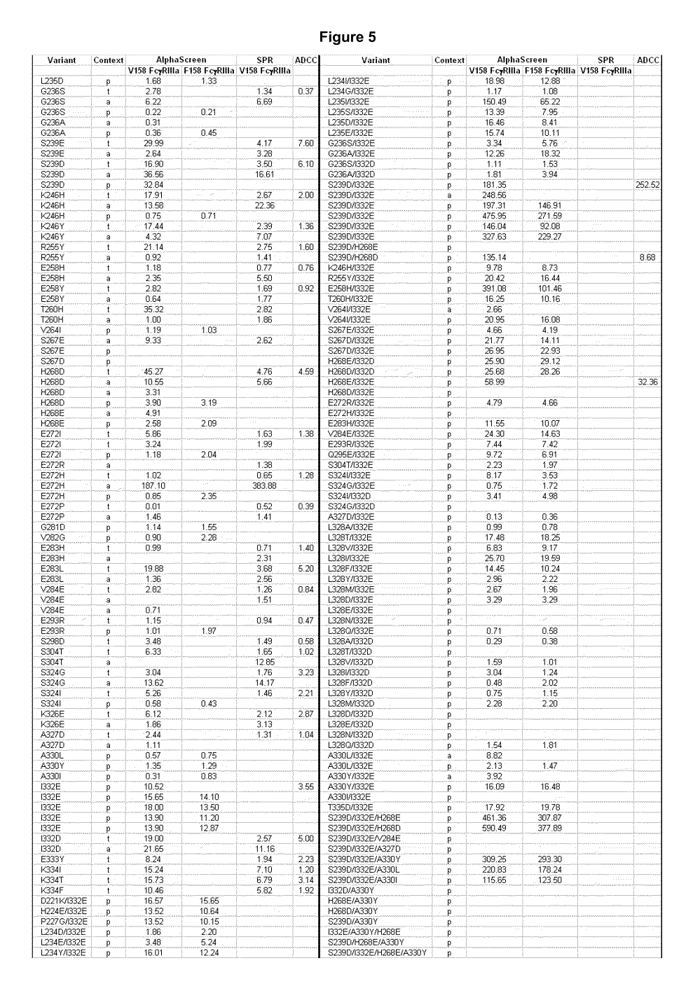

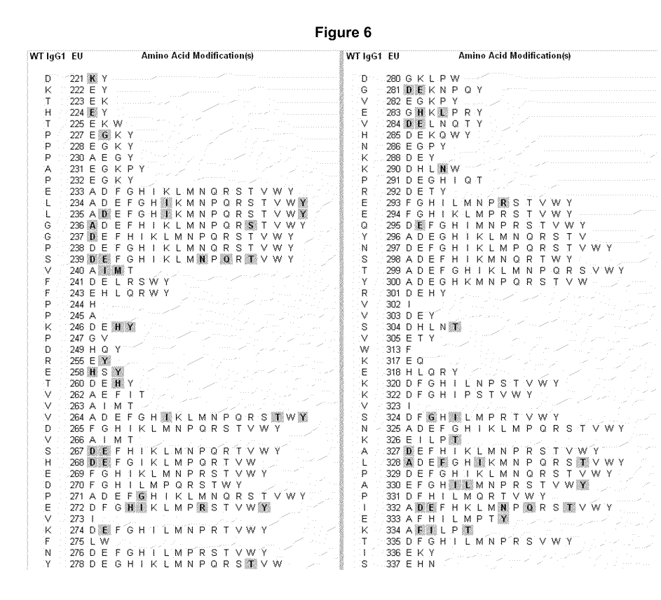

Novel Fc variants have been successfully engineered, primarily in the context of the IgG1 isotype, with selectively enhanced binding to FcγRs, and these variants have been shown to provide enhanced potency and efficacy in cell-based effector function assays (U.S. Ser. No. 10/672,280, U.S. Ser. No. 10/822,231, U.S. Ser. No. 60/627,774, U.S. Ser. No. 60/642,477, and U.S. Ser. No. 60/723,294, entitled “Optimized Fc Variants”, filed Oct. 3, 2005, all expressly incorporated by reference). FIGS. 4 and 5 summarize these variants and the data detailing their properties with respect to Fc ligand affinity and effector function. FIG. 6 summarizes the amino acid modifications that compose this set of variants.

The variants described in FIGS. 4-6 provide a variety of unique biological and clinical properties. A number of variants provide substantial enhancements in FcγR affinity, in particular to one or both isoforms (V158 and F158) of the activating receptor FcγRIIIa. For example substitutions at positions 239, 268, and 332 provide substantial improvements in FcγR binding and effector function. A number of variants have been obtained with altered specificities for the various Fc ligands. The selective affinity of a variant for the different FcγRs may be an important factor in determining the optimal therapeutic IgG. For example, the affinity of a variant for FcγRI, the relative affinity for FcγRIII versus FcγRIIb, and/or the relative affinity for FcγRIIa versus FcγRIIb may be important determinants of the capacity of an antibody or Fc fusion to mediate ADCC or ADCP, or elicit long-term immunity. For example, the balance between FcγRIIa and FcγRIIb establishes a threshold of DC activation and enables immune complexes to mediate opposing effects on dendritic cell (DC) maturation and function (Boruchov et al., 2005, J Clin Invest, Sep. 15, 1-10). Thus variants that selectively ligate FcγRIIa or FcγRIIb may affect DC processing, T cell priming and activation, antigen immunization, and/or efficacy against cancer (Dhodapkar & Dhodapkar, 2005, Proc Natl Acad Sci USA, 102, 6243-6244). Such variants may be employed as novel strategies for targeting antigens to the activating or inhibitory FcγRs on human DCs to generate either antigen-specific immunity or tolerance. Some variants provide selective enhancement in binding affinity to different Fc ligands, whereas other provide selective reduction in binding affinity to different Fc ligands. By “selective enhancement” as used herein is meant an improvement in or a greater improvement in binding affinity of a variant to one or more Fc ligands relative to one or more other Fc ligands. For example, for a given variant, the Fold WT for binding to, say FcγRIIa, may be greater than the Fold WT for binding to, say FcγRIIb. By “selective reduction” as used herein is meant a reduction in or a greater reduction in binding affinity of a variant to one or more Fc ligands relative to one or more other Fc ligands. For example, for a given variant, the Fold WT for binding to, say FcγRI, may be lower than the Fold WT for binding to, say FcγRIIb. As an example of such selectivity, G236S provides a selective enhancement to FcγRII's (IIa, IIb, and IIc) relative to FcγRI and FcγRIIIa, with a somewhat greater enhancement to FcγRIIa relative to FcγRIIb and FcγRIIc. G236A, however, is highly selectively enhanced for FcγRIIa, not only with respect to FcγRI and FcγRIIIa, but also over FcγRIIb and FcγRIIc. Selective enhancements and reductions are observed for a number of Fc variants, including but not limited to variants comprising substitutions at EU positions 234, 235, 236, 267, 268, 292, 293, 295, 300, 324, 327, 328, 330, and 335. In particular, receptor selectivity may be provided by variants comprising one or more substitutions selected from the group consisting of 236S, 236A, 267D, 267E, 268D, 268E, 293R, 324I, 327D, 272R, 328A, 328F, 271G, 235Y, 327D, 328A, 328F, 324G, 330Y, 330L, and 3301. FIG. 6 highlights preferred non-naturally occurring modifications that provide optimized Fc ligand binding and/or effector function properties. Alternately preferred non-naturally occurring modifications include 234Y, 234I, 235Y, 235I, 235D, 236S, 237D, 239D, 239E, 239N, 239Q, 239T, 240M, 246H, 246Y, 255Y, 258Y, 264I, 264T, 264Y, 267D, 267E, 271G, 272Y, 272H, 272R, 272I, 274E, 278T, 283L, 283H, 293R, 324G, 324I, 326T, 327D, 328A, 328F, 328T, 330L, 330Y, 330I, 332D, 332E, 332N, 332Q, 332T, 333Y, 334F, and 334T. Most preferred non-naturally occurring modifications include 234Y, 234I, 235Y, 235I, 235D, 236S, 237D, 239D, 239E, 239N, 239Q, 239T, 264I, 264T, 264Y, 267D, 267E, 324G, 324I, 327D, 328A, 328F, 328T, 330L, 330Y, 330I, 332D, 332E, 332N, 332Q, and 332T.

### Example 2

### IgG Variants with Non-Naturally Occurring Modifications

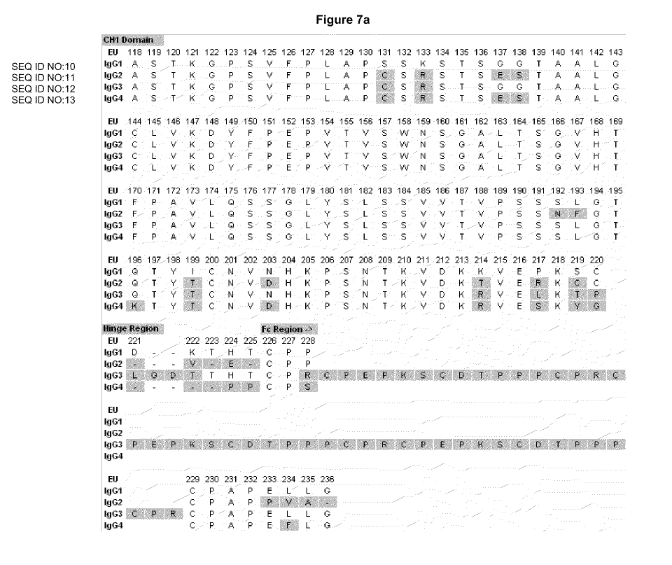

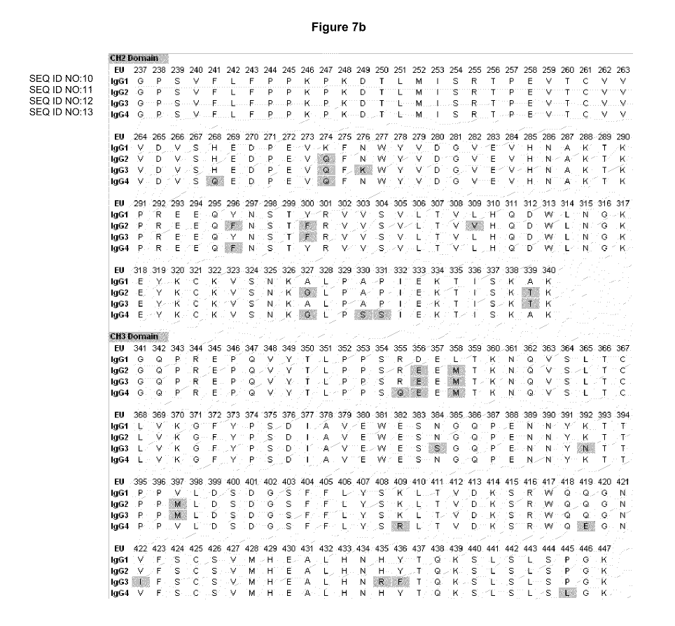

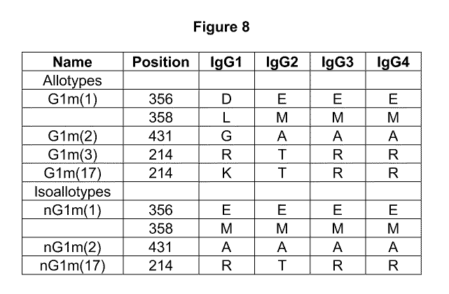

The present invention provides immunoglobulins wherein the aforedescribed novel variants are utilized in the context of alternate IgG isotypes. FIG. 7 shows the sequences of the four IgG isotypes IgG1, IgG2, IgG3, and IgG4, with differences from IgG1 highlighted. Thus FIG. 7 provides the isotypic differences between the four IgGs. For completeness, it is noted that in addition to isotypic differences, a number of immunoglobulin polymorphisms (referred to as Gm polymorphisms) or allotypes exist in the human population. Gm polymorphism is determined by the IGHG1, IGHG2 and IGHG3 genes which have alleles encoding allotypic antigenic determinants referred to as G1m, G2m, and G3m allotypes for markers of the human IgG1, IgG2 and IgG3 molecules (no Gm allotypes have been found on the gamma 4 chain) (Clark, 1997, IgG effector mechanisms, Chem. Immunol. 65:88-110; Gorman & Clark, 1990, Semin Immunol 2(6):457-66). Allelic forms of human immunoglobulins have been well-characterized (WHO Review of the notation for the allotypic and related markers of human immunoglobulins. J Immunogen 1976, 3:357-362; WHO Review of the notation for the allotypic and related markers of human immunoglobulins. 1976, Eur. J. Immunol. 6, 599-601; Loghem E van, 1986, Allotypic markers, Monogr Allergy 19: 40-51). At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b5, b0, b3, b4, s, t, g1, c5, u, v, g5) (Lefranc, et al., The human IgG subclasses: molecular analysis of structure, function and regulation. Pergamon, Oxford, pp. 43-78 (1990); Lefranc, G. et al., 1979, Hum. Genet.: 50, 199-211). Additionally, other polymorphisms have been characterized (Kim et al., 2001, J. Mol. Evol. 54:1-9). As an example, FIG. 8 shows the allotypes and isoallotypes of the gamma1 chain of human IgG1 showing the positions and the relevant amino acid substitutions.

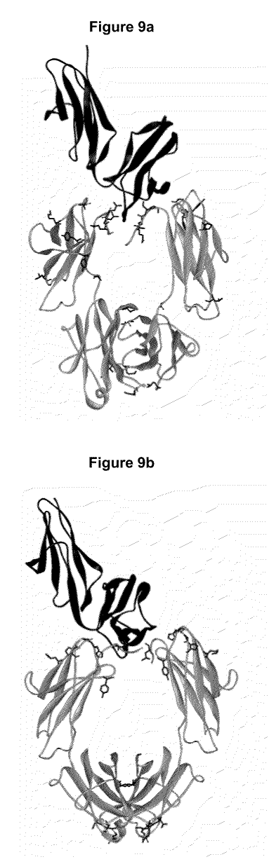

The different IgG isotypes offer a variety of unique physical, biological, and therapeutic properties. For example there are significant differences in stability, solubility, FcγR-mediated effector functions, complement-mediated effector functions, in vivo pharmacokinetics, and oligomerization state among the isotypes IgG1, IgG2, IgG3, and IgG4. These differences must be due to one or more of the isotypic differences between the IgGs shown in FIG. 7. For example, because the binding site for FcγRs resides on the Fc region, it is likely that the IgG differences in Fc, and even more likely the lower hinge and the CH2 domain, are responsible for the differences in their FcγR-mediated effector functions. FIGS. 9a and 9b highlight the differences between the Fc region of IgG1 and those of IgG2 and IgG4 respectively, mapped in the context of the IgG1 Fc/FcγRIIIb complex (pdb accession code 1E4K)(Sondermann et al., 2000, Nature 406:267-273).

In order to explore the properties of the different IgG isotypes, a matched set of IgG1, IgG2, and IgG4 antibodies were constructed with the variable region of the anti-Her2/neu antibody trastuzumab (Herceptin®, a registered trademark of Genentech, currently approved for treatment of breast cancer). The genes for the variable regions of trastuzumab were constructed using recursive PCR, and subcloned into the mammalian expression vector pcDNA3.1Zeo (Invitrogen) comprising the full length light kappa (Cκ) and heavy chain IgG1 constant regions. DNA was sequenced to confirm the fidelity of the sequences. Plasmids containing heavy chain gene (VH-Cγ1-Cγ2-Cγ3) (wild-type or variants) were co-transfected with plasmid containing light chain gene (VL-Cκ) into 293T cells. Media were harvested 5 days after transfection, and antibodies were purified from the supernatant using protein A affinity chromatography (Pierce). Antibody concentrations were determined by bicinchoninic acid (BCA) assay (Pierce).

In order to screen for FcγR binding, the extracellular region of human V158 FcγRIIIa was expressed and purified. The extracellular region of this receptor was obtained by PCR from a clone obtained from the Mammalian Gene Collection (MGC:22630). The receptor was fused at the C-terminus with a 6×His-tag and a GST-tag, and subcloned into pcDNA3.1zeo. Vector containing receptor was transfected into 293T cells, media were harvested, and receptors were purified using Nickel affinity chromatography. Receptor concentrations were determined by bicinchoninic acid (BCA) assay (Pierce). Binding affinity to human FcγRIIIa by the antibodies was measured using a quantitative and extremely sensitive method, AlphaScreen™ assay. The AlphaScreen is a bead-based luminescent proximity assay. Laser excitation of a donor bead excites oxygen, which if sufficiently close to the acceptor bead will generate a cascade of chemiluminescent events, ultimately leading to fluorescence emission at 520-620 nm. The AlphaScreen was applied as a competition assay for screening the antibodies. Commercial IgG was biotinylated by standard methods for attachment to streptavidin donor beads, and tagged human FcγRIIIa (V158 isoform) was bound to glutathione chelate acceptor beads. In the absence of competing antibody, antibody and FcγR interact and produce a signal at 520-620 nm. Addition of untagged antibody competes with the Fc/FcγR interaction, reducing fluorescence quantitatively to enable determination of relative binding affinities.

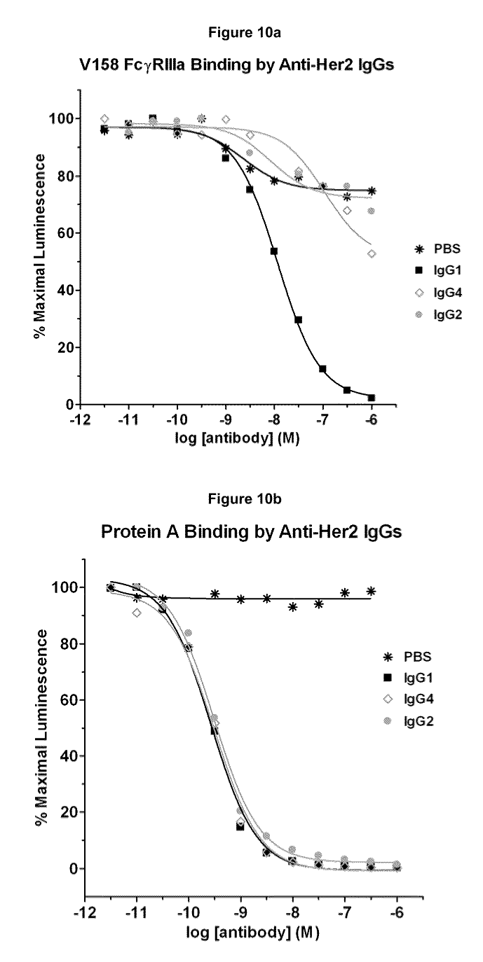

FIG. 10a presents the competition AlphaScreen binding data for binding of trastuzumab IgGs to human V158 FcγRIIIa. The binding data were normalized to the maximum and minimum luminescence signal provided by the baselines at low and high concentrations of competitor antibody respectively. The data were fit to a one site competition model using nonlinear regression, and these fits are represented by the curves in the figure. The results show that the FcγR-mediated effector functions are substantially greater for IgG1 than for IgG2 and IgG4, consistent with prior studies (Michaelsen et al., 1992, Molecular Immunology, 29(3): 319-326). FIG. 10b presents competition AlphaScreen data for binding of the IgGs to protein A, carried out using commercial protein A-conjugated acceptor beads. The data show that all of the variants bind comparably to protein A, indicating that the FcγR-affinity differences are not due to differences in stability, solubility, or other properties between the IgG isotypes.

Non-naturally occurring modifications were constructed in the context of all three antibody isotypes. The substitutions S239D and 1332E were introduced into the heavy chains of the trastuzumab IgG1, IgG2, and IgG4 antibodies using quick-change mutagenesis techniques (Stratagene), and antibodies were expressed and purified as described above. Competition AlphaScreen data were acquired as described above for binding to human V158 FcγRIIIa, as well as human FcγRI, which was constructed using recursive PCR and expressed and purified as described above. FIGS. 11a and 11b show the data for binding of the IgG variants to these receptors. The results show that the novel modifications S239D/1332E provide enhanced receptor binding to all three isotypes, despite the poor FcγR affinity of IgG2 and IgG4 relative to IgG1.

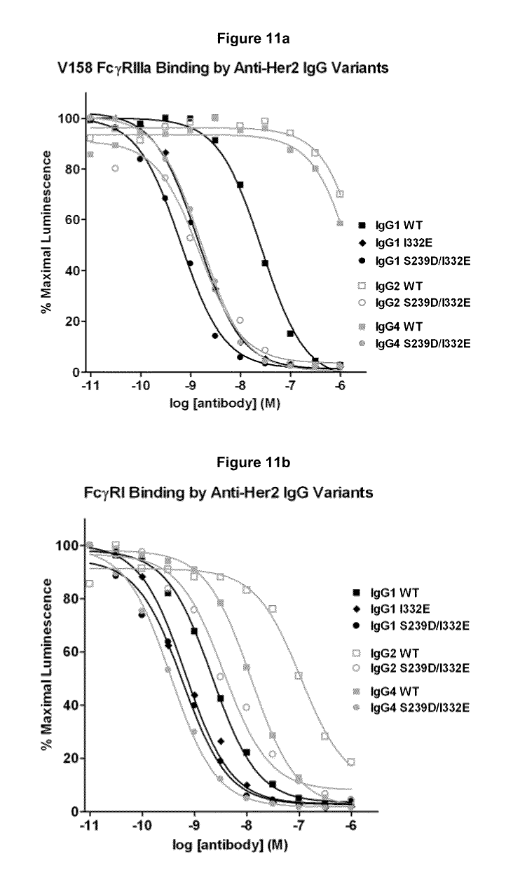

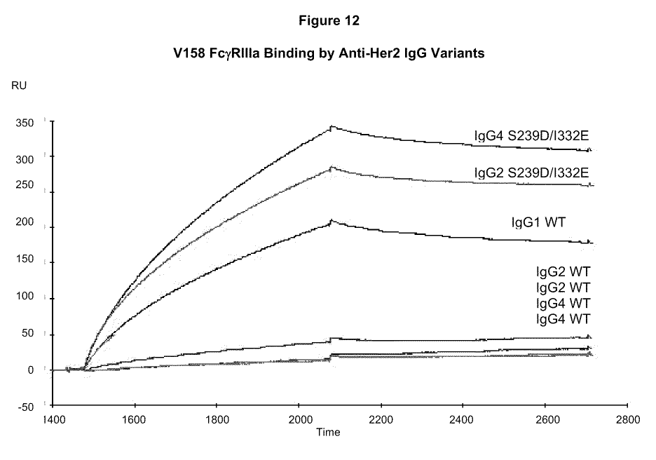

Surface Plasmon Resonance (SPR) (Biacore, Uppsala, Sweden) was carried out to further investigate the FcγRIIIa affinity of the IgG variants. Protein A (Pierce) was covalently coupled to a CM5 sensor chip using NHS/EDC chemistry. WT or variant trastuzumab antibody was bound to the protein A CM5 chip, and FcγRIIIa-His-GST analyte, in serial dilutions was injected (association phase) and washed (dissociation phase). Response in resonance units (RU) was acquired, and data were normalized for baseline response, obtained from a cycle with antibody and buffer alone. FIG. 12 provides the kinetic traces for the binding of WT IgG1, WT IgG2, WT IgG4, S239D/I332E IgG2, and S239D/I332E IgG4 antibodies to human V158 FcγRIIIa. The relative amplitudes of the binding traces reflect the relative FcγR affinities of the variants. The data corroborate the AlphaScreen data, indicating further that the novel modifications provide significant FcγR binding enhancements to IgG2 and IgG4.

### Example 3

### IgGs Variants with Novel and Isotypic Amino Acid Modifications

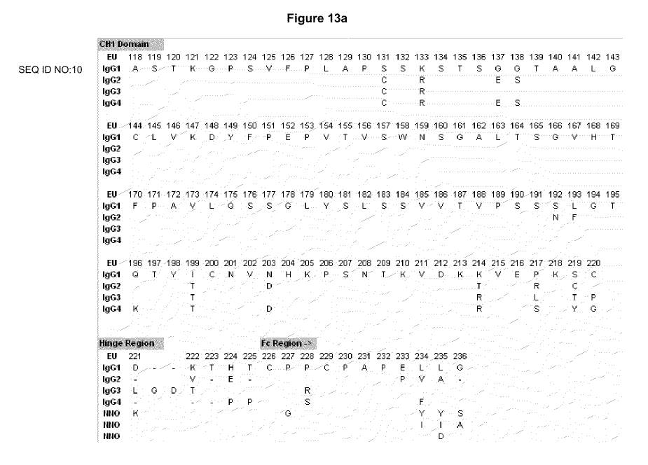

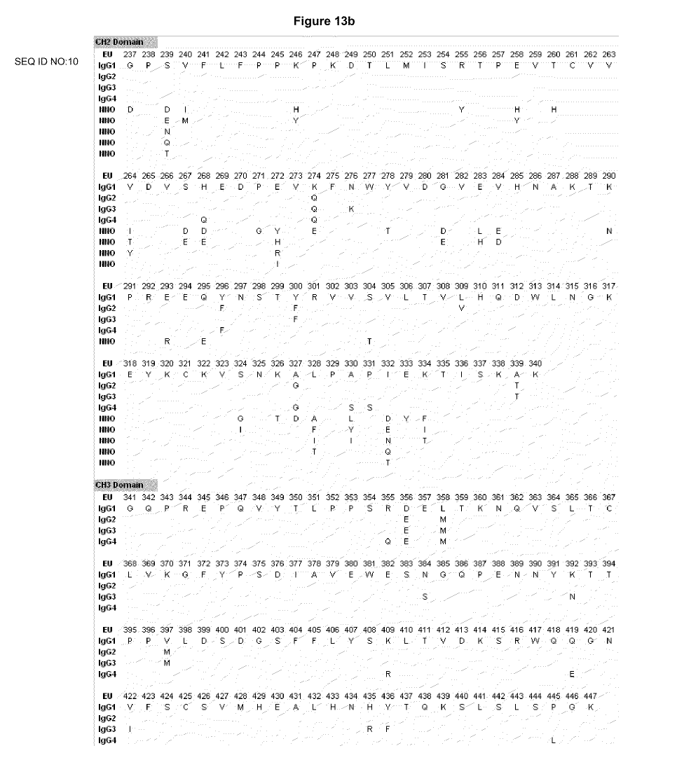

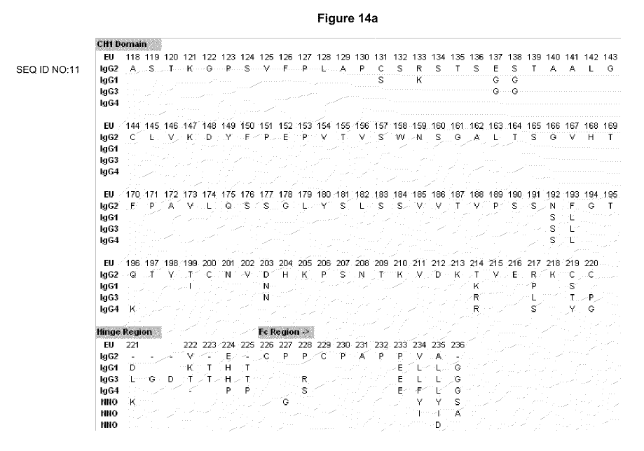

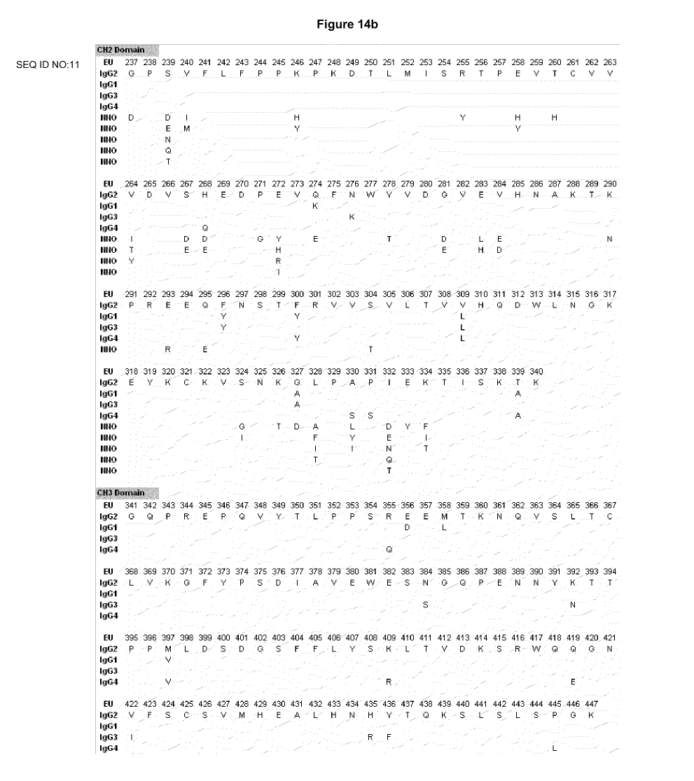

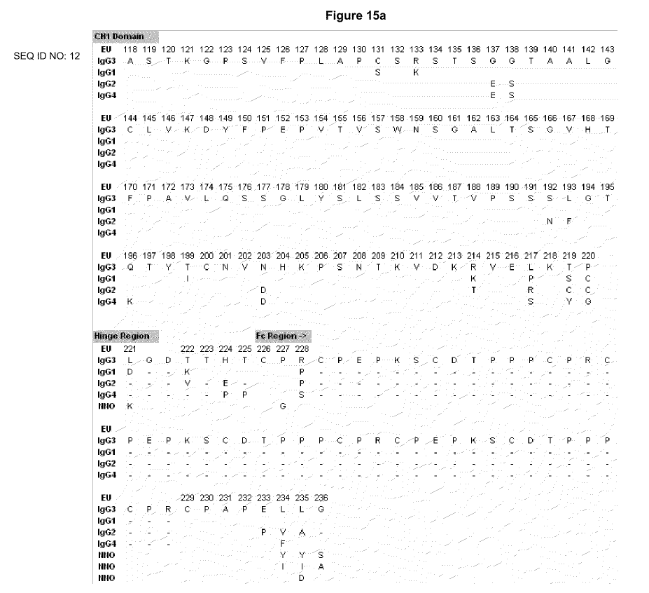

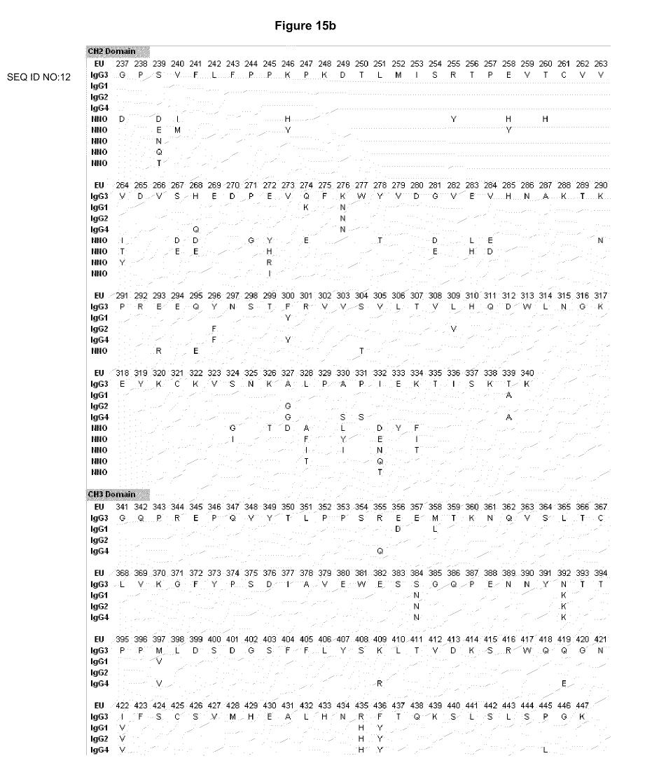

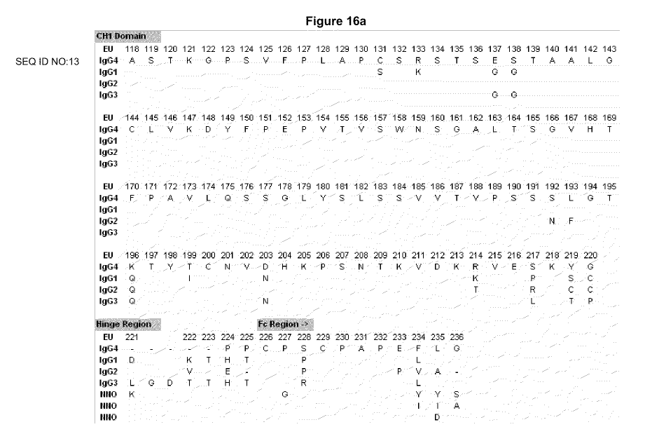

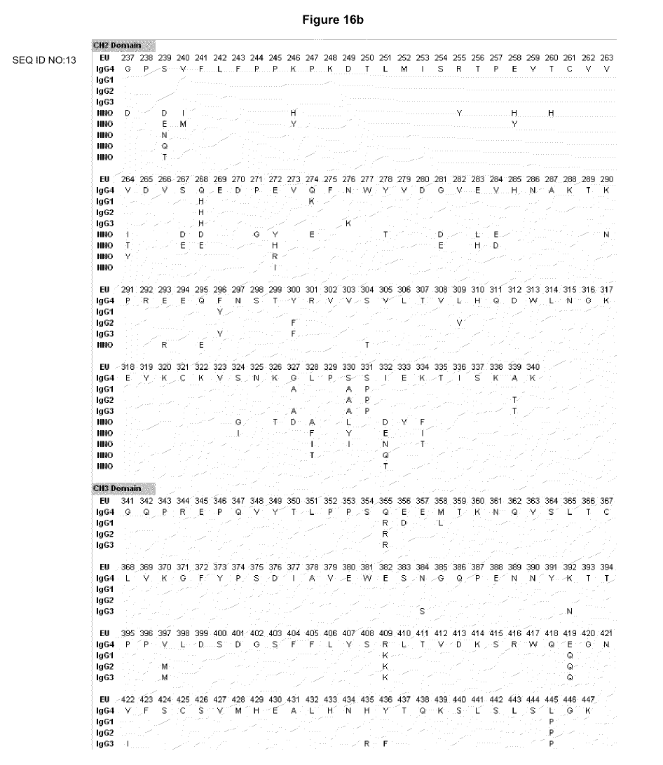

The present invention provides immunoglobulins wherein the aforedescribed novel variants are coupled with isotypic modifications to provide IgG variants with optimized properties. FIGS. 13-16 describe a set of novel and isotypic amino acid modifications for each isotype IgG1 (FIG. 13), IgG2 (FIG. 14), IgG3 (FIG. 15), and IgG4 (FIG. 16). The sequence of the parent IgG is provided explicitly, and novel and isotypic residues are provided at appropriate EU positions according to FIG. 6. As an example in FIG. 14, IgG2 is the parent immunoglobulin and comprises a deletion at EU position 236. IgG1, IgG2, and IgG3 all comprise glycines at position 236, and serine and alanine are two preferred novel substitutions at position 236. Thus FIG. 14 describes in the parent immunoglobulin IgG2 the isotypic modifications -236G and the novel modifications -236S and -236A. According to FIGS. 14 and 6, the full set of novel modifications in the parent IgG2 at position 236 include -236A, -236D, -236E, -236F, -236H, -2361, -236K, -236L, -236M, -236N, -236P, -236Q, -236R, -236S, -236T, -236V, -236W, and -236Y.

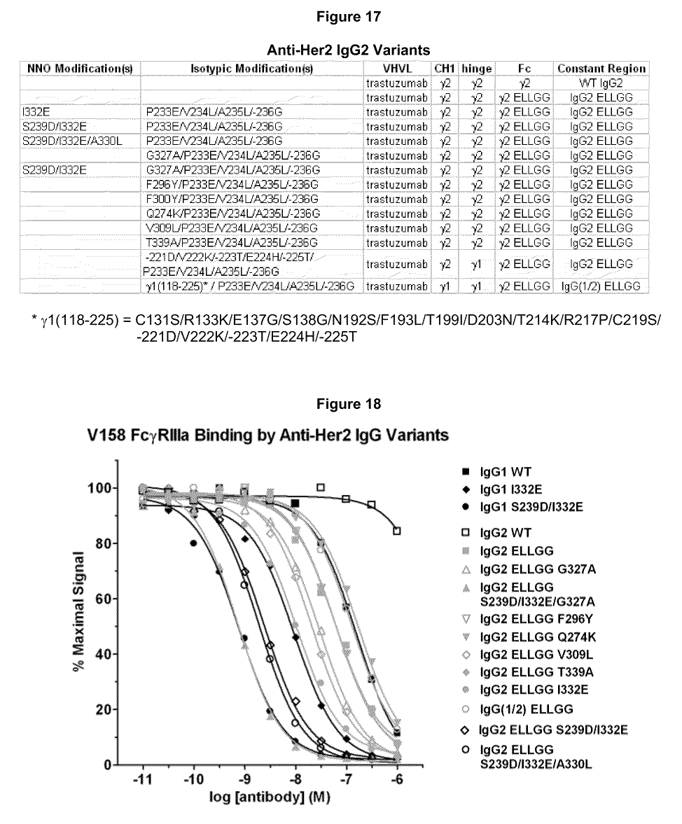

A set of IgG2 trastuzumab variants were constructed comprising novel and isotypic modifications using the information provided in FIG. 14. FIG. 17 provides this set of IgG variants. For simplicity, constant regions are labeled for easy reference. P233E/V234L/A235U-236G IgG2, referred to as IgG2 ELLGG, is an IgG2 variant described previously (Chappel et al., 1991, Proc. Natl. Acad. Sci. USA 88(20):9036-9040; Chappel et al., 1993, Journal of Biological Chemistry 268:25124-25131). γ1(118-225)/P233E/V234L/A235L/-236G IgG2, referred to as IgG(1/2) ELLGG, is a novel IgG2 variant comprising the P233E/V234L/A235L/-236G modifications of IgG2 ELLGG and the full set of IgG2 to IgG1 isotypic modifications in the CH1 domain and hinge region (γ1(118-225)). These variants were constructed, expressed, and purified as described previously. FIG. 18 shows competition AlphaScreen data for binding of the IgG2 trastuzumab variants to human V158 FcγRIIIa, carried out as described. The results show the favorable FcγR binding properties of the IgG2 ELLGG and IgG(1/2) ELLGG variants. Furthermore, the results show that a number of novel and isotypic modifications significantly improve the FcγR binding affinity of the IgG2 isotype.

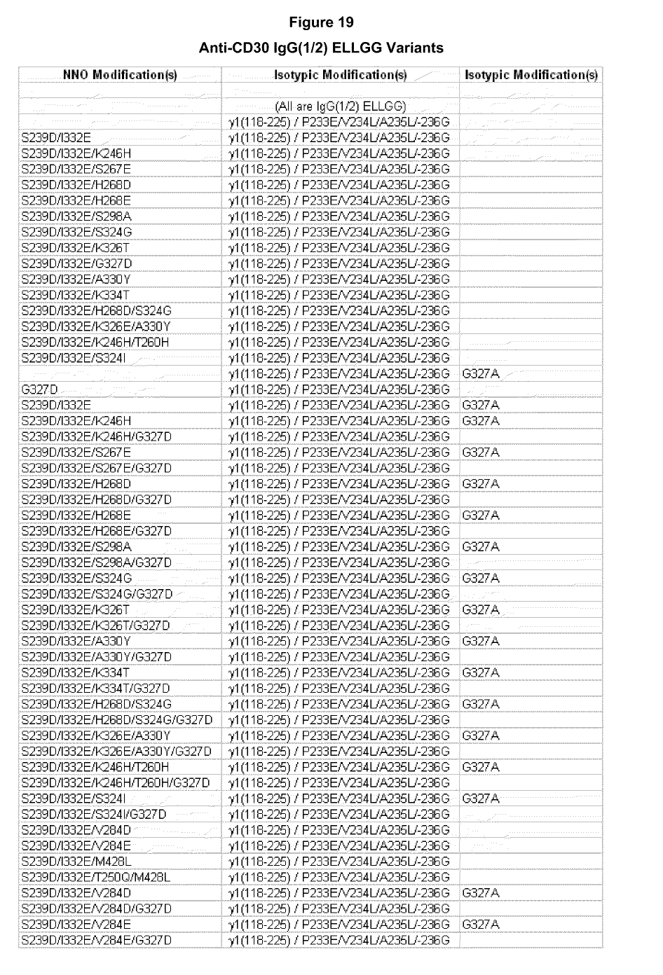

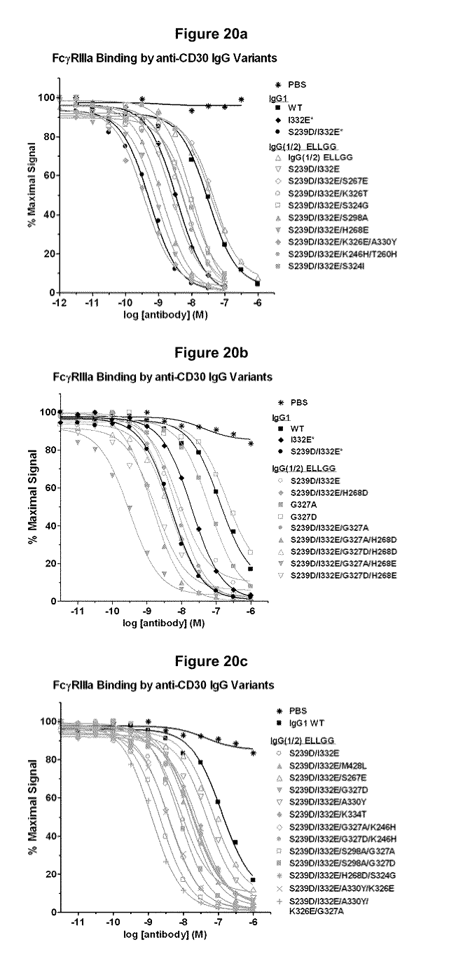

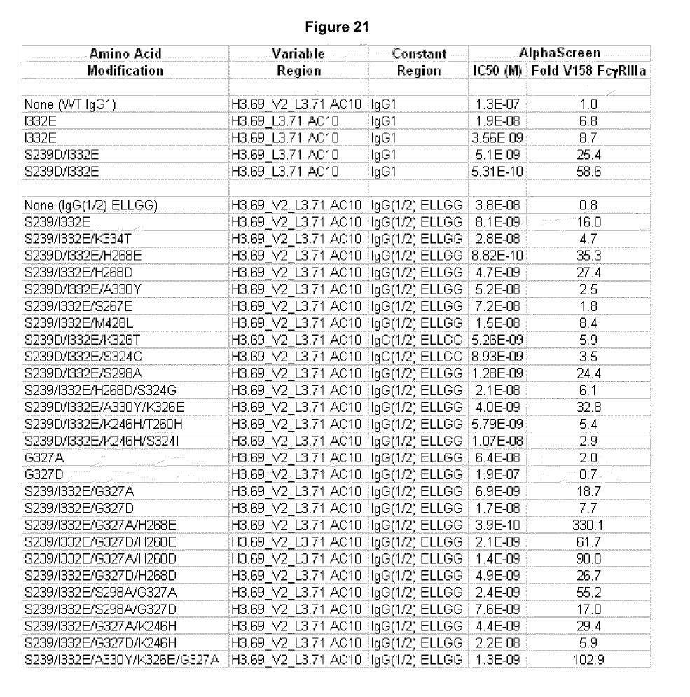

A series of isotypic and novel modifications were made and tested in the context of IgG(1/2) ELLGG to further explore the properties of this IgG variant. These variants are provided in FIG. 19. The variable region of these IgG variants is that of H3.69_V2_L3.69 AC10, which is an anti-CD30 antibody with reduced immunogenicity. H3.69_V2_L3.69 AC10 is a variant of H3.69_L3.71 AC10 described in U.S. Ser. No. 11/004,590 (herein expressly incorporated by reference) with a mutation I2V in the H3.69 VH region. The set of variants in FIG. 19 comprise novel and isotypic modifications in the context of IgG(1/2) ELLGG. These variants were constructed, expressed, and purified as described previously. FIG. 20 shows competition AlphaScreen data for binding of the anti-CD30 IgG2 variants to human V158 FcγRIIIa, carried out as described. The fits to the data provide the inhibitory concentration 50% (IC50) (i.e. the concentration required for 50% inhibition) for each antibody, thus enabling the relative binding affinities of Fc variants to be quantitatively determined. By dividing the IC50 for each variant by that of H3.69_V2_L3.71 AC10 IgG1, the fold-enhancement or reduction in receptor binding (Fold V158 FcγRIIIa) are obtained. These values are provided in FIG. 21. The results further show that the Fc ligand binding properties of the IgG isotypes can be significantly improved via engineering of novel and isotypic amino acid modifications.

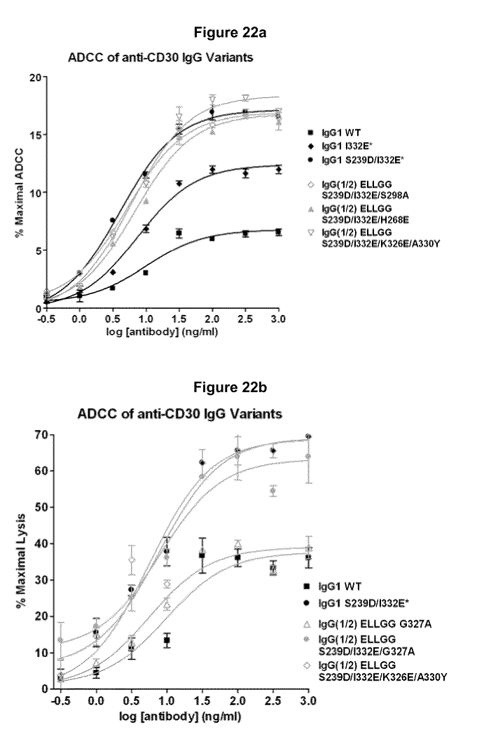

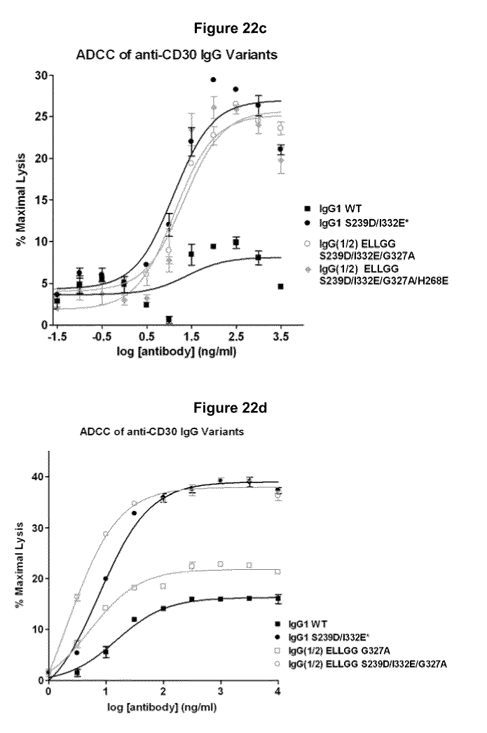

Cell-based ADCC assays were carried out on the anti-CD30 IgG variants to investigate their effector function properties. ADCC was measured using either the DELFIA® EuTDA-based cytotoxicity assay (Perkin Elmer) or LDH Cytotoxicity Detection Kit (Roche Diagnostic Corporation, Indianapolis, Ind.). Human PBMCs were purified from leukopacks using a ficoll gradient. For europium-based detection, target cells were first loaded with BATDA at 1×10⁶ cells/ml and washed 4 times. For both europium- and LDH-based detection, CD30+ L540 Hodgkin's lymphoma target cells were seeded into 96-well plates at 10,000 cells/well, and opsonized using Fc variant or WT antibodies at the indicated final concentration. Triton X100 and PBMCs alone were typically run as controls. Effector cells were added at 25:1 PBMCs:target cells, and the plate was incubated at 37° C. for 4 hrs. Cells were incubated with either Eu3+ solution or LDH reaction mixture, and relative fluorescence units were measured. Data were normalized to maximal (triton) and minimal (PBMCs alone) lysis, and fit to a sigmoidal dose-response model using nonlinear regression. FIG. 22a-22d provide these data. The results show that the optimized FcγR binding properties of the IgG variants result in improved effector function.

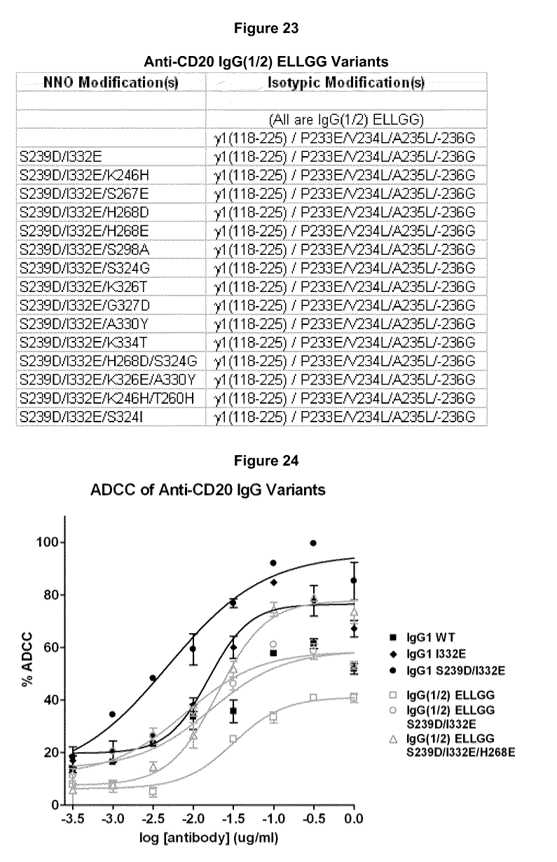

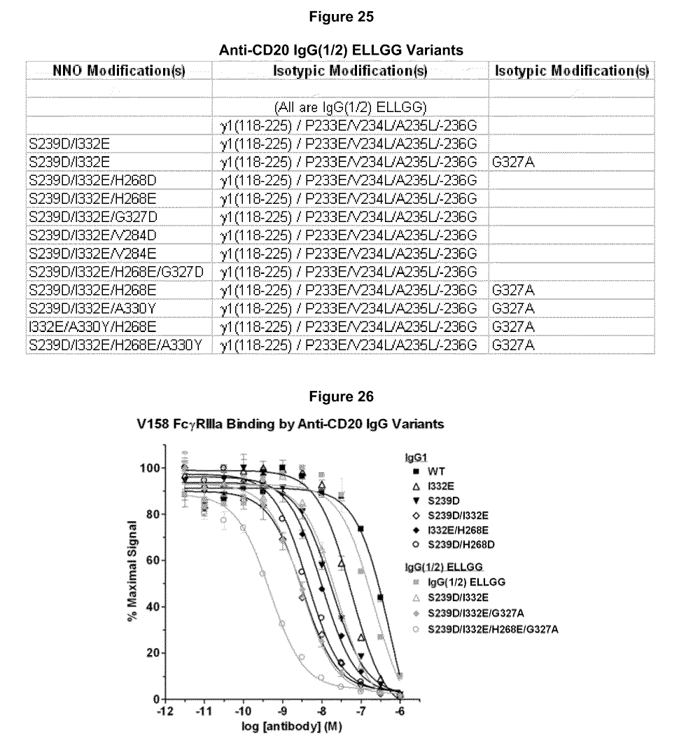

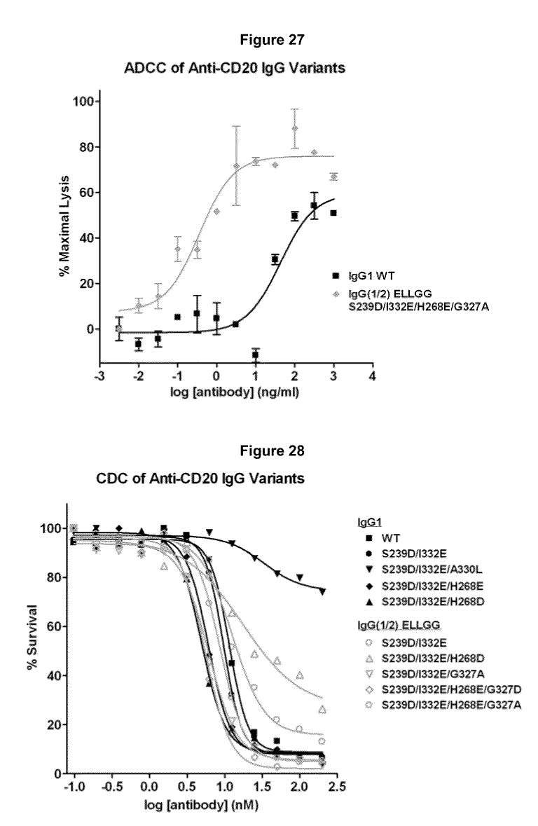

A set of IgG variants comprising novel and isotypic modifications were made and tested in the context of two antibodies that target the B-cell antigen CD20. FIG. 23 provides a set of IgG variants comprising the variable region of C2B8, an anti-CD20 antibody currently marketed as the biotherapeutic rituximab (U.S. Pat. No. 5,736,137). These variants were constructed, expressed, and purified as described previously. FIG. 24 shows cell-based ADCC data for select rituximab IgG2 variants against CD20+ WIL2-S lymphoma target cells. FIG. 25 provides a set of IgG variants comprising the variable region of the anti-CD20 antibody PRO70769 (PCT/US2003/040426). These variants were constructed, expressed, and purified as described previously. FIG. 26 shows competition AlphaScreen data for binding of these anti-CD20 IgG variants to human V158 FcγRIIIa, and FIG. 27 provides a cell-based ADCC for one of the PRO70769 IgG variants against WIL2-S cells. The results are consistent with the aforedescribed results, indicating that the IgG variants are the invention are broadly applicable for improving clinically relevant antibodies.

To explore the effect of the novel and isotypic modifications on complement activity, a cell-based CDC assay was performed. Target WIL2-S lymphoma cells were washed 3× in 10% FBS medium by centrifugation and resuspension, and seeded at 50,000 cells/well. Anti-CD20 antibodies was added at the indicated final concentrations. Human serum complement (Quidel, San Diego, Calif.) was diluted 50% with medium and added to antibody-opsonized target cells. Final complement concentration was approximately ⅙^{th} original stock. Plates were incubated for 2 hrs at 37° C., Alamar Blue was added, and cells were cultured for two days. Fluorescence was measured, and data were normalized to the maximum and minimum signal and fit to a sigmoidal dose-response curve. FIG. 28 shows these data. The results indicate that the novel and isotypic modifications of the invention can be further employed to modulate IgG CDC activity.

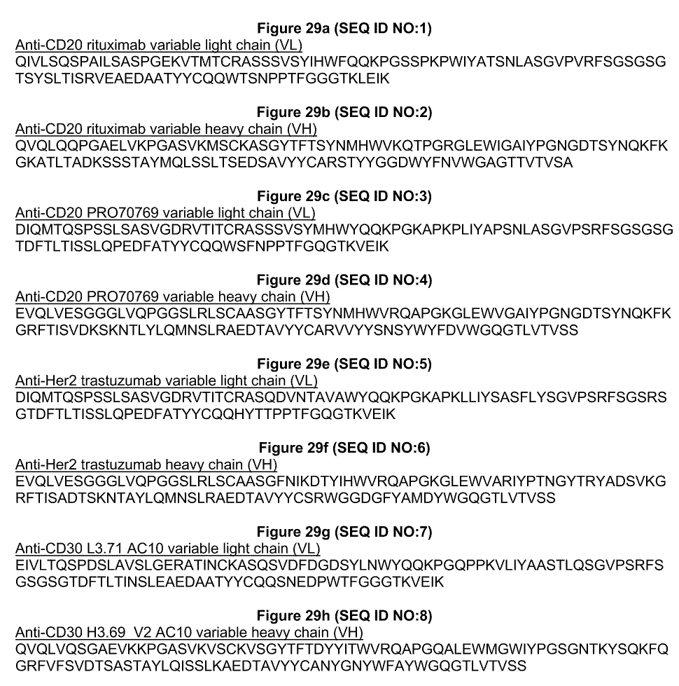

FIG. 29 provides the amino acid sequences of the variable region VL and VH domains utilized in the present invention, including the anti-CD20, anti-Her2, and anti-CD30 antibodies. These sequences are not meant to constrain the present invention to these variable regions. The present invention contemplates application of the described IgG variants to other antibodies that target CD20, Her2, and CD30. Particularly preferred are anti-CD20 antibodies that bind to an identical or overlapping CD20 epitope as C2B8, anti-CD20 antibodies that bind to an identical or overlapping CD20 epitope as PRO70769, anti-Her2 antibodies that bind to an identical or overlapping Her2 epitope as trastuzumab, and anti-CD30 antibodies that bind to an identical or overlapping CD30 epitope as H3.69_V2_L3.71 AC10. The present invention of course contemplates application of the described IgG variants to antibodies that target other antigens besides CD20, Her2, and CD30.

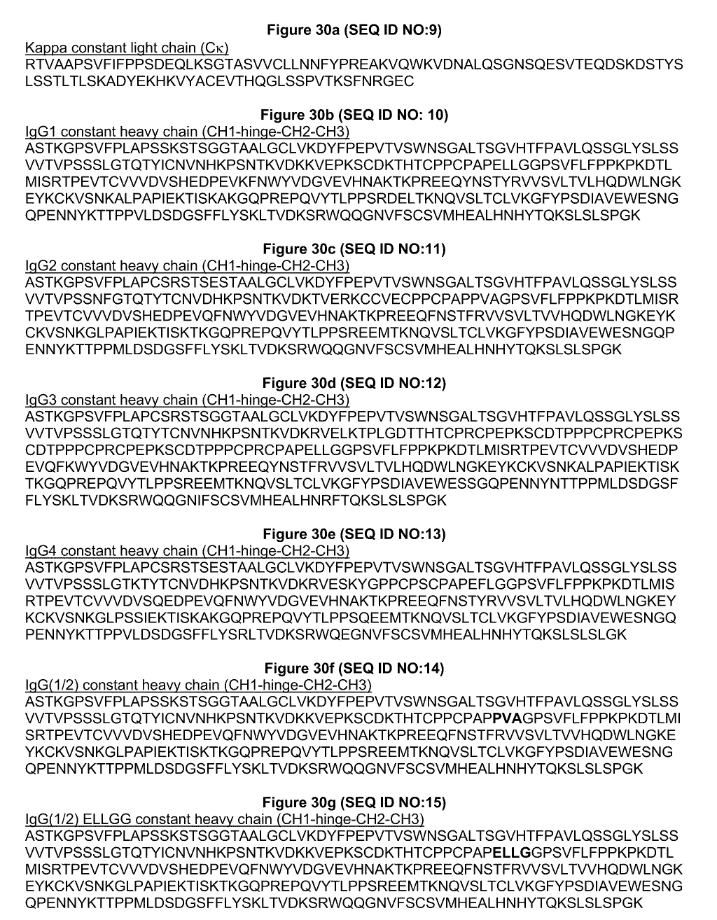

FIG. 30 provides the constant region amino acid sequences described in the present invention. These include the constant light chain kappa region, the four IgG isotypes IgG1, IgG2, IgG3, and IgG4, the IgG2 ELLGG constant region, and the IgG(1/2) ELLGG constant region. These sequences are not meant to constrain the present invention to these constant regions. For example, although the kappa constant chain (Cκ) was used in the present study, the lambda constant chain (Cλ) may be employed.

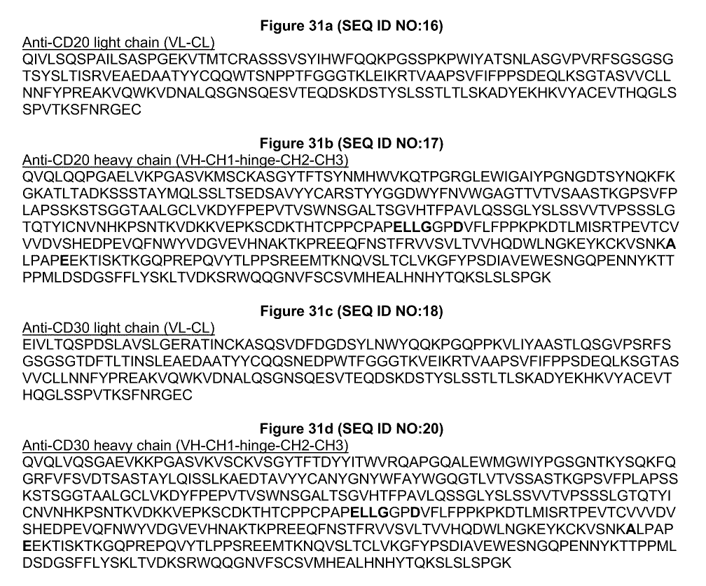

FIGS. 31a and 31b provide the amino acid sequences of the full length light and heavy chains of one of the anti-CD20 IgG variants described in the present invention. FIGS. 31c and 31d provide the amino acid sequences of the full length light and heavy chains of one of the anti-CD30 IgG variant described in the present invention.

All references are herein expressly incorporated by reference.

Whereas particular embodiments of the invention have been described above for purposes of illustration, it will be appreciated by those skilled in the art that numerous variations of the details may be made without departing from the invention as described in the appended claims.

We claim:

1. An IgG variant comprising an amino acid sequence having the formula:
ASTKGPSVFPLAPCSRSTSESTAALGCLVKDYFPEPVTVSWNSGALTSGVHTFPAVLQSSG
LYSLSSVVTVPSSNFGTQTYTCNVDHKPSNTKVDKTVERKCC-X(221)-X(222)-X(223)-
X(224)-X(225)-C-X(227)-X(228)-C-X(230)-X(231)-X(232)-ELLGG-X(238)-X(239)-X(240)-
X(241)-L-X(243)-X(244)-X(245)-X(246)-X(247)-K-X(249)-TLMIS-X(255)-TP-X(258)-V-
X(260)-C-X(262)-X(263)-X(264)-X(265)-X(266)-X(267)-X(268)-X(269)-X(270)-X(271)-
X(272)-X(273)-X(274)-X(275)-X(276)-W-X(278)-V-X(280)-X(281)-X(282)-X(283)-X(284)-
X(285)-X(286)-A-X(288)-T-X(290)-X(291)-X(292)-X(293)-X(294)-X(295)-X(296)-X(297)-
X(298)-X(299)-X(300)-X(301)-X(302)-X(303)-X(304)-X(305)-LTVVHQD-X(313)-LNG-X(317)-
X(318)-Y-X(320)-C-X(322)-X(323)-X(324)-X(325)-X(326)-X(327)-X(328)-X(329)-X(330)-
X(331)-X(332)-X(333)-X(334)-X(335)-X(336)-X(337)-
KTKGQPREPQVYTLPPSREEMTKNQVSLTCLVKGFYPSDIAVEWESNGQPENNYKTTPPM
LDSDGSFFLYSKLTVDKSRWQQGNVFSCSVMHEALHNHYTQKSLSLSPGK-,
wherein
-X(221)- is selected from the group consisting of no amino acid, K and Y;
-X(222)- is selected from the group consisting of V, E and Y;
-X(223)- is selected from the group consisting of no amino acid, E and K;
-X(224)- is selected from the group consisting of E and Y;
-X(225)- is selected from the group consisting of no amino acid, E, K and W;
-X(227)- is selected from the group consisting of P, E, G, K and Y;
-X(228)- is selected from the group consisting of P, E, G, K and Y;
-X(230)- is selected from the group consisting of P, A, E, G and Y;
-X(231)- is selected from the group consisting of A, E, G, K, P and Y;
-X(232)- is selected from the group consisting of P, E, G, K and Y;
-X(238)- is selected from the group consisting of P, D, E, F, G, H, I, K, L, M, N, Q, R, S, T, V, W and Y;
-X(239)- is selected from the group consisting of S, D, E, F, G, H, I, K, L, M, N, P, Q, R, T, V, W and Y;
-X(240)- is selected from the group consisting of V, A, I, M and T;
-X(241)- is selected from the group consisting of F, D, E, L, R, S, W and Y;
-X(243)- is selected from the group consisting of F, E, H, L, Q, R, W and Y;
-X(244)- is selected from the group consisting of P and H;
-X(245)- is selected from the group consisting of P and A;
-X(246)- is selected from the group consisting of K, D, E, H and Y;
-X(247)- is selected from the group consisting of P, G and V;
-X(249)- is selected from the group consisting of D, H, Q and Y;
-X(255)- is selected from the group consisting of R, E and Y;
-X(258)- is selected from the group consisting of E, H, S and Y;
-X(260)- is selected from the group consisting of T, D, E, H and Y;
-X(262)- is selected from the group consisting of V, A, E, F, I and T;
-X(263)- is selected from the group consisting of V, A, I, M and T;
-X(264)- is selected from the group consisting of V, A, D, E, F, G, H, I, K, L, M, N, P, Q, R, S, T, W and Y;
-X(265)- is selected from the group consisting of D, F, G, H, I, K, L, M, P, Q, R, S, T, V, W and Y;
-X(266)- is selected from the group consisting of V, A, I, M and T;
-X(267)- is selected from the group consisting of S, D, E, F, H, I, K, L, M, N, P, Q, R, V, W and Y;
-X(268)- is selected from the group consisting of H, D, E, F, G, I, K, L, M, P, R, T, V and W;
-X(269)- is selected from the group consisting of E, F, G, H, I, K, L, M, N, P, R, S, T, V, W and Y;
-X(270)- is selected from the group consisting of D, F, G, H, I, L, M, P, Q, R, S, T, W and Y;
-X(271)- is selected from the group consisting of P, A, D, E, F, G, H, I, K, L, M, N, Q, R, S, T, V, W and Y;
-X(272)- is selected from the group consisting of E, D, F, G, H, I, K, L, M, P, R, S, T, V, W and Y;
-X(273)- is selected from the group consisting of V and I;
-X(274)- is selected from the group consisting of Q, D, E, F, G, H, I, L, M, N, P, R, T, V, W and Y;
-X(275)- is selected from the group consisting of F, L and W;
-X(276)- is selected from the group consisting of N, D, E, F, G, H, I, L, M, P, R, S, T, V, W and Y;
-X(278)- is selected from the group consisting of Y, D, E, G, H, I, K, L, M, N, P, Q, R, S, T, V and W;
-X(280)- is selected from the group consisting of D, G, K, L, P and W;
-X(281)- is selected from the group consisting of G, D, E, K, N, P, Q and Y;
-X(282)- is selected from the group consisting of V, E, G, K, P and Y;
-X(283)- is selected from the group consisting of E, G, H, K, L, P, R and Y;
-X(284)- is selected from the group consisting of V, D, E, L, N, Q, T and Y;
-X(285)- is selected from the group consisting of H, D, E, K, Q, W and Y;
-X(286)- is selected from the group consisting of N, E, G, P and Y;
-X(288)- is selected from the group consisting of K, D, E and Y;
-X(290)- is selected from the group consisting of K, D, H, L, N and W;
-X(291)- is selected from the group consisting of P, D, E, G, H, I, Q and T;
-X(292)- is selected from the group consisting of R, D, E, T and Y;
-X(293)- is selected from the group consisting of E, F, G, H, I, L, M, N, P, R, S, T, V, W and Y;
-X(294)- is selected from the group consisting of E, F, G, H, I, K, L, M, P, R, S, T, V, W and Y;
-X(295)- is selected from the group consisting of Q, D, E, F, G, H, I, M, N, P, R, S, T, V, W and Y;
-X(296)- is selected from the group consisting of F, A, D, E, G, I, K, L, M, N, Q, R, S, T and V;
-X(297)- is selected from the group consisting of N, D, E, F, G, H, I, K, L, M, P, Q, R, S, T, V, W and Y;
-X(298)- is selected from the group consisting of S, E, F, H, I, K, M, Q, R, W and Y;
-X(299)- is selected from the group consisting of T, A, D, E, F, G, H, I, K, L, M, N, P, Q, R, S, V, W and Y;
-X(300)- is selected from the group consisting of F, A, D, E, G, H, K, M, N, P, Q, R, S, T, V and W;
-X(301)- is selected from the group consisting of R, D, E, H and Y;
-X(302)- is selected from the group consisting of V and I;
-X(303)- is selected from the group consisting of V, D, E and Y;
-X(304)- is selected from the group consisting of S, D, H, L, N and T;
-X(305)- is selected from the group consisting of V, E, T and Y;
-X(313)- is selected from the group consisting of W and F;
-X(317)- is selected from the group consisting of K, E and Q;
-X(318)- is selected from the group consisting of E, H, L, Q, R and Y;
-X(320)- is selected from the group consisting of K, D, F, G, H, I, L, N, P, S, T, V, W and Y;
-X(322)- is selected from the group consisting of K, D, F, G, H, I, P, S, T, V, W and Y;
-X(323)- is selected from the group consisting of V and I;
-X(324)- is selected from the group consisting of S, D, F, G, H, I, L, M, P, R, T, V, W and Y;
-X(325)- is selected from the group consisting of N, A, D, E, F, G, H, I, K, L, M, P, Q, R, S, T, V, W and Y;
-X(326)- is selected from the group consisting of K, I, L, P and T;
-X(327)- is selected from the group consisting of A, G, D, E, F, H, I, K, L, M, N, P, R, T, V, W and Y;
-X(328)- is selected from the group consisting of L, A, D, E, F, G, H, I, K, M, N, P, Q, R, S, T, V, W and Y;
-X(329)- is selected from the group consisting of P, D, E, F, G, H, I, K, L, M, N, Q, R, S, T, V, W and Y;
-X(330)- is selected from the group consisting of A, E, F, G, H, I, L, M, N, P, R, T, V, W and Y;
-X(331)- is selected from the group consisting of P, D, F, H, I, L, M, Q, R, T, V, W and Y;
-X(332)- is selected from the group consisting of I, A, D, E, F, H, K, L, M, N, P, Q, R, S, T, V, W and Y;
-X(333)- is selected from the group consisting of E, F, H, I, L, M, P, T and Y;
-X(334)- is selected from the group consisting of K, F, I, P and T;
-X(335)- is selected from the group consisting of T, D, F, G, H, I, L, M, N, P, R, S, V, W and Y;
-X(336)- is selected from the group consisting of I, E, K and Y;
-X(337)- is selected from the group consisting of S, E, H and N, and
wherein the sequence differs from SEQ ID No: 15 by at least a single amino acid and numbering is according to the EU index.

2. The IgG variant according to claim 1, wherein the X(327) is A.

3. The IgG variant according to claim 2, wherein the X(239) is D and the X(332) is E.

4. The IgG variant according to claim 2, wherein X(239) is D.

5. The IgG variant according to claim 2, wherein X(332) is E.
